(19) United States
(12) Patent Application Publication
(10) Pub. No.: US 2025/0213627 A1
(43) Pub. Date: Jul. 3, 2025

(54) RECOMBINANT MICROORGANISM-BASED METHODS AND COMPOSITIONS FOR TREATMENT OF DISEASE

(71) Applicant: Baylor College of Medicine, Houston, TX (US)

(72) Inventors: Robert Britton, Houston, TX (US); Joseph Hyser, Houston, TX (US); Christine Beeton, Houston, TX (US)

(73) Assignee: Baylor College of Medicine, Houston, TX (US)

(21) Appl. No.: 18/576,307

(22) PCT Filed: Jul. 8, 2022
(86) PCT No.: PCT/US22/73569

§ 371 Date: Jan. 3, 2024

Related U.S. Application Data
(60) Provisional application No. 63/219,717, filed on Jul. 8, 2021.

(51) Int. Cl. A61K 35/744; A61K 38/16; A61K 45/06; A61P 19/02; C07K 14/435; C12N 1/20; C12R 1/225

(52) U.S. Cl. CPC A61K 35/744; A61K 38/16; A61K 45/06; A61P 19/02; C07K 14/43504; C07K 14/43522; C12N 1/20; C12R 2001/225

(57) ABSTRACT

Aspects of the disclosure concern methods and compositions related to recombinant microorganisms expressing one or more biologically active peptides that are heterologous to the microorganism, wherein the one or more biologically active peptides comprise a potassium channel blocker. In particular aspects, recombinant microorganisms are generated by introducing into the microorganism one or more polynucleotides encoding one or more biologically active peptides. In specific aspects, a subject having one or more diseases or conditions mediated by cells of the immune system and/or fibroblasts or fibroblast-like cells is administered a therapeutically effective amount of a recombinant microorganism or composition thereof, wherein the recombinant microorganism is capable of producing a therapeutically effective amount of the one or more biologically active polypeptides to treat or prevent the one or more diseases or conditions mediated by cells of the immune system and/or fibroblasts or fibroblast-like cells.

### CROSS-REFERENCE TO RELATED APPLICATIONS

This application is a national phase application under 35 U.S.C. § 371 that claims priority to International Application No. PCT/US2022/073569 filed Jul. 8, 2022, which claims priority to U.S. Provisional Application Ser. No. 63/219,717, filed Jul. 8, 2021, both of which are incorporated by reference herein in their entirety.

### INCORPORATION OF SEQUENCE LISTING

The instant application contains a Sequence Listing, named “SeqLst_BAYMP0338US.xml” (23,874 bytes; created Aug. 8, 2024) which has been submitted electronically in XML format and is hereby incorporated by reference in its entirety.

### BACKGROUND

### I. Field of the Disclosure

Aspects of the disclosure concern at least the fields of cell biology, molecular biology, biochemistry, immunology, gastroenterology, and medicine.

### II. Background

Biologics now constitute a significant element of available medical treatments for various conditions such as chronic inflammatory disorders, cancer and metabolic disease. Nearly 30% of all drugs approved by the US. Food and Drug Administration in 2015-2018 were biologics, yet, the majority biologics are administered via parenteral route because of poor bioavailability via oral route. Fear of needles, injection-associated infection and pain are responsible for skipping doses by patients, especially for those with chronic inflammatory diseases that often requires life-long treatment.

According to the National Institute of Allergy and Infectious Diseases, more than 80 autoimmune diseases, such as rheumatoid arthritis (RA), type 1 diabetes, and multiple sclerosis, affect ˜50 million people in the US alone, causing symptoms ranging from mild discomfort to severe disability, and can result in death if not adequately controlled. RA alone affects ˜1.5 million people in the US. Although current therapeutics have considerably improved the management of RA, RA-induced reduction in lifespan has not improved and may be worsening²⁰. Current RA treatments focus on improving inflammation and pain, and include nonsteroidal anti-inflammatory drugs (NSAIDs), corticosteroids, disease-modifying anti-rheumatic drugs (DMARDs) such as methotrexate, and biologic response modifiers such as antibodies targeted to cytokines and their receptors (TNFα or IL-1β for example). These treatments focus on improving inflammation and pain control. However, they have little effect on the pathogenesis of RA and many increase the probability of infections or cancer. Additionally, current treatments require weekly or more frequent injections, and can lead to missed doses and poor patient compliance, resulting in inconsistent or sub-therapeutic levels of the drug. Further, many patients experience significant side effects, including increased risk of infections and cancer and kidney or liver damage, leading 30-50% of patients to alter their treatment regimens^{21,22}. All current treatments have side effects, due at least in part to the systemic nature of delivery of the treatments that range from enhanced risk of severe infections to cancer. Furthermore, the biologics can be immunogenic, leading to the generation of neutralizing antibodies after repeated drug administration.

The present disclosure satisfies a long-felt need in the art by providing novel recombinant organism-based therapeutics that combine efficacy with limited side effects and a non-invasive method for delivery to treat one or more diseases or conditions, including but not limited to one or more diseases or conditions that may be mediated by cells of the immune system and/or fibroblasts or fibroblast-like cells.

### SUMMARY

The current disclosure fulfills the need in the art by providing methods and compositions for treating or preventing one or more diseases or conditions, including but not limited to diseases or conditions that may be mediated by cells of the immune system and/or fibroblasts or fibroblast-like cells. In some aspects, the one or more diseases or conditions have inflammation as at least one symptom. In particular aspects, a subject in need of treatment and/or prevention of a disease or condition is provided a therapeutically effective amount of a recombinant microorganism expressing one or more biologically active peptides that are heterologous to the microorganism or a composition comprising said recombinant microorganism. In some aspects, administration of such a recombinant microorganism or composition thereof results in inhibition of the proliferation and/or activation of immune cells, fibroblasts, and/or fibroblast-like cells thereby treating or preventing one or more diseases or conditions, including but not limited to diseases or conditions that may be mediated by cells of the immune system and/or fibroblasts or fibroblast-like cells, by reducing inflammation associated therewith.

Aspects of the disclosure include methods for treating one or more diseases or conditions, methods for treating one or more disorders mediated by cells of the immune system and/or fibroblasts or fibroblast-like cells, methods for preventing one or more diseases or conditions, methods for preventing one or more disorders mediated by cells of the immune system and/or fibroblasts or fibroblast-like cells, methods for reducing the severity of one or more diseases or conditions, methods for reducing the severity of one or more disorders mediated by cells of the immune system and/or fibroblasts or fibroblast-like cells, methods for delaying the onset of one or more diseases or conditions, methods for delaying the onset of one or more disorders mediated by cells of the immune system and/or fibroblasts or fibroblast-like cells, and methods for generating recombinant microorganisms for use in methods of treating, preventing, reducing the severity of, and/or delaying the onset of one or more diseases or conditions, including but not limited to one or more disorders mediated by cells of the immune system and/or fibroblasts or fibroblast-like cells.

Methods of the present disclosure can include at least 1, 2, 3, 4, 5, or more of the following steps: introducing one or more polynucleotides encoding one or more biologically active peptides into a microorganism to generate a recombinant microorganism, administering one or more recombinant microorganisms expressing one or more biologically active peptides to a subject, administering compositions comprising recombinant microorganisms expressing one or more biologically active peptides to a subject, determining a subject to have a higher risk of developing one or more diseases or conditions, determining that one or more diseases or conditions pose a greater risk to the health or life of the subject, determining a subject to have a disease or condition, diagnosing a subject with one or more diseases or conditions, determining a subject to have a higher risk of developing one or more disorders mediated by cells of the immune system and/or fibroblasts or fibroblast-like cells, determining that one or more disorders mediated by cells of the immune system and/or fibroblasts or fibroblast-like cells pose a greater risk to the health or life of the subject, determining a subject to have one or more disorders mediated by cells of the immune system and/or fibroblasts or fibroblast-like cells, determining a subject to have one or more disorders mediated by cells of the immune system and/or fibroblasts or fibroblast-like cells characterized at least by inflammation, determining a subject having one or more disorders mediated by cells of the immune system and/or fibroblasts or fibroblast-like cells to have inflammation, determining a subject having one or more disorders mediated by cells of the immune system and/or fibroblasts or fibroblast-like cells to have inflammation caused at least in part by the proliferation and/or activation of immune cells and/or fibroblasts or fibroblast-like cells, diagnosing a subject with one or more disorders mediated by cells of the immune system and/or fibroblasts or fibroblast-like cells, and inhibiting the proliferation and/or activation of immune cells, fibroblasts, and/or fibroblast-like cells in the subject to improve one or more diseases or conditions, including one or more disorders mediated by cells of the immune system and/or fibroblasts or fibroblast-like cells in a subject. It is contemplated that any one or more of these steps may be excluded from certain aspects of the disclosure.

Compositions of the present disclosure can include at least 1, 2, 3, or more of the following components: a recombinant organism, nonsteroidal anti-inflammatory drugs, one or more corticosteroids, one or more disease-modifying drugs, one or more anti-rheumatic drugs, one or more antibodies targeted to cytokines or cytokine receptors, a pharmaceutically acceptable carrier, and a pharmaceutical excipient. The composition may be comprised in a pharmaceutical composition, a food product, a nutraceutical, and/or a probiotic. It is contemplated that any one or more of these components may be excluded from certain aspects of the disclosure.

In specific aspects, the recombinant organism is a non-commensal, non-colonizing microorganism. In specific aspects, the microorganism is a non-pathogenic microorganism. In specific aspects, wherein the microorganism is a probiotic microorganism. In specific aspects, the microorganism is a gram-positive bacterium. In particular aspects, the microorganism is a Lactobacillus species strain. In particular aspects, the Lactobacillus species is Lactobacillus reuteri. In particular aspects, the Lactobacillus species is L. reuteri strain 6475. In particular aspects, the L. reuteri strain 6475 is LJ01.

In specific aspects, the one or more biologically active peptides comprise one or more immunomodulatory peptides. In some aspects, the one or more immunomodulatory peptides comprise one or more potassium channel blockers. In some aspects, the potassium channel blockers inhibit one or more potassium channels upregulated by activated immune cells. In some aspects, the immune cells are CCR7⁻ effector memory T-cells (T_{EM} cells), and activation of T_{EM} cells induces the upregulation of one or more potassium channels by the T_{EM} cells. In some aspects, the immune cells are class-switched memory B cells, and activation of class-switched memory B cells induces the upregulation of one or more potassium channels by the class-switched memory B cells. In some aspects, the one or more potassium channels inhibited by the potassium channel blockers and/or upregulated by activated T_{EM} cells and/or class-switched memory B cells comprise the Kv1.3 potassium channel.

In other aspects, the one or more biologically active peptides comprise one or more non-immunomodulatory peptides. In some aspects, the one or more non-immunomodulatory peptides comprise one or more potassium channel blockers. In some aspects, the potassium channel blockers inhibit one or more potassium channels expressed by fibroblasts or fibroblast-like cells. In some aspects, the fibroblast-like cells are fibroblast-like synoviocytes (FLS), and inhibition of one or more potassium channels inhibits the proliferation, invasiveness, and secretion of cytokines, chemokines, proteases, and growth factors by FLS without inducing cell death. In some aspects, the one or more potassium channels inhibited by the potassium channel blockers in FLS comprise the KCa1.1 potassium channel.

In specific aspects, the one or more biologically active peptides comprise one or more derivatives of dalazatide (ShK-186). In some aspects, the one or more derivatives of dalazatide comprise ShK-235. In some aspects, the one or more biologically active peptides are secreted by the recombinant microorganism.

Disclosed herein, in some aspects, is a recombinant microorganism expressing one or more biologically active peptides that are heterologous to the microorganism, wherein the one or more biologically active peptides comprise one or more potassium channel blockers. In some aspects, the microorganism is a non-pathogenic microorganism. In some aspects, the microorganism is a probiotic microorganism. In some aspects, the microorganism is a gram-positive bacterium. In some aspects, the microorganism is a Lactobacillus species strain. In some aspects, the microorganism is a Lactobacillus reuteri strain. In some aspects, the microorganism is Lactobacillus reuteri ATCC PTA 6475 or a derivative thereof. In some aspects, the microorganism is Lactobacillus reuteri LJ01. In some aspects, the one or more biologically active peptides are secreted by the recombinant microorganism.

In some aspects, the one or more biologically active peptides inhibit one or more potassium channels upregulated by activated immune cells. In these aspects, the activated immune cells can comprise CCR7⁻ effector memory T-cells and/or IgD⁻ class-switched memory B-cells, the potassium channel can comprise a Kv1.3 potassium channel, the one or more biologically active peptides can comprise dalazatide or one or more derivatives thereof, and the one or more derivatives of dalazatide comprise ShK-235.

In some aspects, the potassium channel blocker inhibits one or more potassium channels upregulated by activated fibroblasts and/or fibroblast-like cells, which can be fibroblast-like synoviocytes, the one or more potassium channels comprise a KCa1.1 potassium channel, and the one or more biologically active peptides comprise iberiotoxin or one or more derivatives thereof.

Also disclosed herein, in some aspects, is a method of generating a recombinant microorganism described herein, the method comprising introducing into the microorganism one or more polynucleotides encoding the one or more biologically active peptides. In some aspects, the one or more polynucleotides encoding the one or more biologically active peptides are under the control of one or more regulatory sequences. In some aspects, the one or more regulatory sequences comprise one or more promoter sequences, enhancer sequences, and/or terminator fragments. In some aspects, the one or more polynucleotides encoding the one or more biologically active peptides comprise a secretory signal sequence upstream of the one or more biologically active polypeptides. In some aspects, the secretory signal sequence comprises a usp45 secretion peptide having at least 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO:1, SEQ ID NO:2, SEQ ID NO:3, SEQ ID NO:4, SEQ ID NO:5, SEQ ID NO:6, or SEQ ID NO:7. In some aspects, the secretory signal sequence comprises a usp45 secretion peptide having at least 95% sequence identity with SEQ ID NO:1. In some aspects, the secretory signal sequence comprises a usp45 secretion peptide comprising SEQ ID NO:1. In some aspects, the secretory signal sequence consists of SEQ ID NO:1.

In some aspects of the method, the method further comprises administration of an effective amount of the recombinant microorganism to an individual in need thereof. In some aspects, the effective amount of the recombinant microorganism comprises at least about 1×10⁷ CFU to at least about 1×10¹² CFU of the recombinant microorganism. In some aspects, the effective amount of the recombinant microorganism comprises at least about 5×10⁹ CFU of the recombinant microorganism.

In some aspects, following administration of the recombinant microorganism to the individual, the recombinant microorganism secretes the one or more biologically active polypeptides in situ in the gastrointestinal tract of the individual. In some aspects, the recombinant microorganism constitutively secretes the one or more biologically active polypeptides. In some aspects, the secreted biologically active polypeptides enter systemic circulation of the individual. In some aspects, the secreted biologically active polypeptides comprise a concentration of at least about 1 nM to at least about 10 nM in the systemic circulation of the individual. In some aspects, the secreted biologically active polypeptides treat or prevent one or more diseases or conditions mediated by cells of the immune system and/or fibroblasts or fibroblast-like cells. In some aspects, the secreted biologically active polypeptides inhibit the proliferation and/or activation of CCR7⁻ effector memory T-cells and/or IgD⁻ class-switched memory B-cells, and the CCR7⁻ effector memory T-cells and/or IgD⁻ class-switched memory B-cells are specific for autoantigens. In some aspects, the secreted biologically active polypeptides inhibit the proliferation and/or activation of fibroblasts and/or fibroblast-like cells. In some aspects, the fibroblast-like cells comprise fibroblast-like synoviocytes.

Also disclosed herein, in some aspects, is a composition compromising a recombinant microorganism described herein. In some aspects, the recombinant microorganism is produced by a method described herein. In some aspects, the composition comprises at least about 1×10⁷ CFU to at least about 1×10¹² CFU of the recombinant microorganism. In some aspects, the composition comprises at least about 5×10⁹ CFU of the recombinant microorganism. In some aspects, the composition comprises a pharmaceutical composition, a food product, a nutraceutical, and/or a probiotic. In some aspects, the composition is comprised in a pharmaceutical composition, a food product, a nutraceutical, and/or a probiotic. In some aspects, the composition further comprises one or more pharmaceutically acceptable carriers and/or excipients.

Also disclosed herein, in some aspects, is a method of treating or preventing one or more diseases or conditions mediated by cells of the immune system and/or fibroblasts or fibroblast-like cells, the method comprising administering to a subject in need thereof a therapeutically effective amount of the recombinant microorganism described herein or the composition described herein, wherein the recombinant microorganism is capable of producing a therapeutically effective amount of the one or more biologically active polypeptides to treat or prevent the one or more diseases or conditions mediated by cells of the immune system and/or fibroblasts or fibroblast-like cells.

In some aspects, the one or more biologically active polypeptides produced by the recombinant microorganism are secreted by the recombinant microorganism. In some aspects, the recombinant microorganism secretes the one or more biologically active polypeptides in situ in the gastrointestinal tract of the individual. In some aspects, the recombinant microorganism constitutively secretes the one or more biologically active polypeptides. In some aspects, the secreted biologically active polypeptides enter systemic circulation of the individual. In some aspects, the secreted biologically active polypeptides comprise a concentration of at least about 1 nM to at least about 10 nM in the systemic circulation of the individual.

In some aspects, the one or more diseases or conditions mediated by cells of the immune system and/or fibroblasts or fibroblast-like cells are characterized by at least one symptom, the at least one symptom comprising inflammation. In some aspects, the inflammation is caused at least in part by proliferation and/or activation of CCR7⁻ effector memory T-cells and/or IgD⁻ class-switched memory B-cells, and the one or more biologically active polypeptides secreted by the recombinant microorganism inhibit the proliferation and/or activation of CCR7⁻ effector memory T-cells and/or IgD⁻ class-switched memory B-cells. In some aspects, the CCR7⁻ effector memory T-cells and/or IgD⁻ class-switched memory B-cells are specific for autoantigens. In some aspects, the inflammation is caused at least in part by proliferation and/or activation of fibroblasts and/or fibroblast-like cells, and the one or more biologically active polypeptides secreted by the recombinant microorganism inhibits the proliferation and/or activation of fibroblasts and/or fibroblast-like cells. In some aspects, the fibroblast-like cells comprise fibroblast-like synoviocytes.

In some aspects, administration of one or more biologically active polypeptides reduces inflammation. In some aspects, the one or more diseases or conditions mediated by cells of the immune system and/or fibroblasts or fibroblast-like cells comprise an inflammatory disease, an autoimmune disease, an alloimmune disease, or an allergic disease. In some aspects, the one or more diseases or conditions mediated by cells of the immune system and/or fibroblasts or fibroblast-like cells comprise a T-cell-mediated disorder and/or a B-cell mediated disorder. In some aspects, the one or more diseases or conditions mediated by cells of the immune system and/or fibroblasts or fibroblast-like cells comprise a fibroblast or fibroblast-like cell-mediated disorder. In some aspects, the one or more diseases or conditions mediated by cells of the immune system and/or fibroblasts or fibroblast-like cells comprise acne vulgaris, allergy, food allergy, atherosclerosis, asthma, allergic asthma, autoimmune diseases, autoinflammatory diseases, celiac disease, chronic inflammatory demyelinating polyneuropathy, chronic prostatitis, colitis, diverticulitis, familial Mediterranean fever, glomerulonephritis, Graves' disease, Guillain-Barre syndrome, Hashimoto's thyroiditis, hidradenitis suppurativa, hypersensitivities, inflammatory bowel diseases, interstitial cystitis, leukocyte defects, lichen planus, mast cell activation syndrome, mastocytosis, multiple sclerosis, myasthenia gravis, myopathy, otitis, pelvic inflammatory disease, pneumonia, psoriasis, reperfusion injury, rheumatic fever, rheumatoid arthritis, rhinitis, sarcoidosis, Sjorgen's syndrome, reperfusion injury, rheumatic fever, rheumatoid arthritis, rhinitis, sarcoidosis, systemic lupus erythematosus, thyroiditis, transplant rejection (e.g., graft versus host disease), type I diabetes, vasculitis, and uveitis. In some aspects, the one or more diseases or conditions mediated by cells of the immune system and/or fibroblasts or fibroblast-like cells comprise rheumatoid arthritis. In some aspects, the one or more diseases or conditions mediated by cells of the immune system and/or fibroblasts or fibroblast-like cells comprise psoriasis.

In some aspects, a recombinant microorganism or a composition described herein is administered orally or rectally. In some aspects, the recombinant microorganism or the composition is administered orally via a pill, spray, capsule, aerosol, lozenge, bolus, tablet, sachet, liquid, suspension, or emulsion. In some aspects, the recombinant microorganism or the composition is administered rectally via a suppository, enema, foam, suspension, or ointment. In some aspects, the recombinant microorganism or the composition is administered at least once a day, at least twice a day, or at least 3 times a day. In some aspects, the recombinant microorganism or the composition is administered at least once a day. In some aspects, the recombinant microorganism is administered in a dose of at least about 1×10⁷ CFU to at least about 1×10¹² CFU of the recombinant microorganism at least once per day. In some aspects, the recombinant microorganism is administered in a dose of at least about 5×10⁹ CFU of the recombinant microorganism at least once per day.

In some aspects, a recombinant microorganism or a composition described herein is administered in combination with one or more additional therapeutics. In some aspects, the one or more additional therapeutics comprise one or more nonsteroidal anti-inflammatory drugs, one or more corticosteroids, one or more disease-modifying anti-rheumatic drugs, or one or more antibodies targeted to cytokines or cytokine receptors.

It is specifically contemplated that any limitation discussed with respect to one aspect of the disclosure may apply to any other aspect of the disclosure. Furthermore, any composition of the disclosure may be used in any method of the disclosure, and any method of the disclosure may be used to produce or to utilize any composition of the disclosure. Aspects of an aspect set forth in the Examples are also aspects that may be implemented in the context of aspects discussed elsewhere in a different Example or elsewhere in the application, such as in the Summary, Detailed Description, Claims, and Description of the Drawings.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIG. 1. Kv1.3 versus KCa3.1 channel numbers per cell in CCR7⁺ naïve, CCR7⁺ T_{CM}, and CCR7⁻ T_{EM} cells before and after activation. Each data point is the mean±SEM channel number in 20-50 cells.

FIGS. 2A-2C. Kv1.3 blockers preferentially inhibit T_{EM} cells while sparing naïve/T_{CM} cells. FIGS. 2A-2B. Naïve/T_{CM} (▪) and T_{EM} (▪) cells were separated based on CCR7 expression and activated in the presence of ShK-186, either immediately after isolation (FIG. 2A) or after an overnight pre-activation to induce the upregulation of either KV1.3 or KCa3.1 (FIG. 2B). FIG. 2C. T_{EM} cells were activated with 10 (◯), 30 (▪), or 90 (▴) μg/ml antigen in the presence of ShK-186. In all aspects, T-cell proliferation was assessed via [3H] thymidine incorporation into the DNA of the dividing cells. Each data point represents mean±SD of triplicates.

FIGS. 3A-3B. Design of the inducible expression system. FIG. 3A. Codon optimized ShK235 with signal peptide Usp45 was ligated into NcoI-XhoI digested pSIP411 resulting in secretion expression plasmid pLL01. FIG. 3B. Amino acid sequences of ShK, its synthetic analog ShK-186, and its recombinant analog ShK-235. Differences between sequences are shown in bold and red. ShK-186 contains a 9-carbon atom linker (AEEA) that precludes its recombinant production. ShK is the 37 amino acid peptide originally isolated from the venom of the sea anemone Stichodactyla helianthus. ShK-186 contains a pTyr attached to the peptide's N-terminus via a 9-carbon atom linker (AEEA) that precludes its recombinant production. ShK-235 differs from ShK by a Q16K substitution, an I21M substitution, and the addition of an Ala to the C-terminus.

FIG. 4. ShK-186/dalazatide displays efficacy in active plaque psoriasis. Psoriasis lesion of a patient before (left) and after 4 weeks of treatment with 60 μg/patient of ShK-186 three times weekly subcutaneously in the periumbilical area (right).

FIGS. 5A-5E. ShK-235 has a high affinity and selectivity for Kv1.3. FIG. 5A. Whole-cell Kv1.3 currents before (control) and after addition of 100 pM ShK-235. FIG. 5B. Dose-response effects of ShK-235 on Kv1.3 currents (n=4 cells per concentration). FIG. 5C. Dose-response of mKv1.3 block by synthetic ShK-235. N=3 cells per concentration. IC50=69.31±24.0 pM. FIG. 5D. Representative traces of the block of mKv1.3 currents by 100 pM synthetic ShK-235. FIG. 5E. Selectivity of ShK-235 against Kv1.x channels determined by whole-cell patch-clamp.

FIGS. 6A-6D. Supernatants from LrS235, but not from LrGusA, block Kv1.3 currents and inhibit the proliferation of human CCR7⁻ T_{EM} cells. FIG. 6A. Representative whole-cell recordings of L929 cells stably expressing mKv1.3 before (control) and after the addition of supernatants diluted 1/10 of LrGusA or LrS235. FIG. 6B. Percentage of remaining Kv1.3 currents after addition of LrGusA (▴) or LrS235 (▾) supernatants diluted 1/10. Mean±SEM, each data point represents a different measurement. FIG. 6C. Representative flow cytometry plots of CELLTRACE™ Violet dye dilution and CCR7 expression of CD3⁺ cells from human peripheral blood mononuclear cells stimulated for 7 days without any bacterial supernatants (left) or in the presence of supernatants from LrGusA (middle) or LrS235 (right). FIG. 6D. Percent of divided human CCR7⁻ T_{EM} and CCR7⁺ naïve/T_{CM} cells in the absence of stimulation (◯) and after anti-CD3 stimulation in the presence of Lr medium (▪) or supernatants of LrGusA (▴) or LrS235 (▾) diluted 1/100. Mean±SEM, N=4 different buffy coat donors. *p<0.05, **p<0.01.

FIGS. 7A-7E. LrS235 reduces disease severity in a pilot trial of collagen-induced arthritis (CIA). FIG. 7A. Clinical scores of paw inflammation from rats with CIA treated over 21 days starting at disease onset with subcutaneous injections of vehicle (□) or 100 μg/kg ShK-235 (▪) every other day, or by daily oral gavage with 1×10⁹ CFU LrGusA (▴) or LrS235 (▾). CIA was induced by two injections at a week interval of porcine collagen II in emulsion in incomplete Freund's adjuvant at the base of the tail. Rats were monitored daily for clinical signs of CIA with a 1 given for each red or swollen toe or knuckle joint, a 2 for each mildly swollen ankle or wrist, and a 5 for each severely swollen ankle or wrist. The maximum score is 15 per paw, or 60 per rat. Data are shown as mean±SEM. The inventors performed a Friedman's test (non-parametric repeated measures one-way ANOVA. *p<0.05, ***p<0.01. FIG. 7B. Joint histology. At the end of the pilot trial, joints sections were stained with hematoxylin and eosin to detect inflammatory infiltrates (arrow heads). Black arrows point to the cartilage. Scale bars: 100 μm. FIGS. 7C-7D. Hematoxylin & eosin (H&E, FIG. 7C, left) and safranin O/fast green (FIG. 7C, right) staining and histology scoring (FIG. 7D) of joints from paws from CIA rats receiving different treatments. Original magnification, 10×, scale bars, 100 μm. FIG. 7E. Representative micro-CT of paws from CIA rats treated with vehicle, synthetic ShK-235 every other day, or oral gavage with LrGusA, LrS235 daily. Data presented as mean±SEM. N=7 to 10 rats per group. Asterisks indicate areas of cartilage erosions. *P<0.05; **P<0.01, ***P<0.001, ****P<0.0001.

FIGS. 8A-8D. LrS235 secretes sufficient ShK-235 in the intestines for detection in the circulation of healthy rats. Healthy rats received an oral bolus of 1×10⁹ CFU of induced LrGusA (▴) or induced LrS235 (▾), or an enteric-coated capsule filled with ShK-235 (●, 2 mg/kg body weight) or gelatin (◯). Blood was drawn at different time points and single-cell patch-clamp used to assess the ability of the serum to block Kv1.3 currents. N=3 rats per time point. FIG. 8A. Representative traces before (control) and after addition of serum diluted 1/100 from the 6-hour time point. FIG. 8B. Current block of serum samples collected at the 6-hour time point. N=3-12 cells. FIG. 8C. Current block of serum samples collected at the indicated time points. Each data point represents a different measurement. FIG. 8D. An active DTH reaction was induced against ovalbumin and rats received a single bolus of the following immediately before ear challenge: 1×10⁹ CFU of LrGusA (▴) or LrS235 (▾) orally, an enteric-coated capsule filled with ShK-235 (●, 2 mg/kg body weight) or gelatin (◯) orally, 1 ml of LrS235 culture supernatant orally (∇), or subcutaneous injection of 0.1 mg/kg synthetic ShK-235 (▪) or vehicle (□). N=6 rats per group (3 males, 3 females). **p<0.01, ***p<0.001.

FIGS. 9A-9B. Circulating half-life of active ShK determined by patch-clamp electrophysiology. FIG. 9A. Rat serum was spiked with known concentrations of ShK, diluted 1/10 in patch-clamp external solution, and assayed for block of Kv1.3 channels by patch-clamp to draw a dose-dependent curve. N=3; mean±SEM. FIG. 9B. Serum concentration of ShK at different time points after a single injection of 80 μg/kg. N=6 rats; mean±SEM.

FIGS. 10A-10B. LrS235-HA secretes functionally active HA-tagged ShK-235. FIG. 10A. Representative whole-cell Kv1.3 currents measured by patch-clamp in cells stably expressing Kv1.3 before (control, top trace) and after application of ShK-235-HA (bottom trace). FIG. 10B. Healthy Lewis rats received a subcutaneous injection of either ShK-235-HA (left) or of untagged ShK-235 (right). Their spleens were collected 3 hours later and stained to detect the HA tag in brown (arrows). Scale bar: 50 μm.

FIG. 11. Immunogenicity of ShK-235 in rats with CIA. The inventors coated wells of an ELISA plate with 1 μg/ml of either ShK-235, rat IgG, or ovalbumin before incubation with serum from the CIA rats treated with vehicle (vertical hatching), LrGusA (left diagonal hatching), LrS235 (right diagonal hatching), or ShK-235 (horizontal hatching) in FIG. 8, diluted 1:100, 1:1000, and 1:10000 and detection of bound IgG using anti-rat IgG-HRP and TMB substrate, and detection at 450 nm. The readings of wells coated with rat IgG all were 3.3 (detector saturation level) and those of wells coated with ovalbumin 1:100 1:1000 1:10000 were <0.2. Mean±SEM of N=3.

FIGS. 12A-12E. Blocking KCa1.1 reduces the severity of an animal model of RA. FIG. 12A. Effects of vehicle (□), paxilline (▪, 20 mg/kg), and iberiotoxin (IbTX, ▪, 0.5 mg/kg) in rats with PIA. Injections were given intraperitoneally (paxilline) or subcutaneously (IbTX), every other day, starting at onset of clinical signs. N=18 rats per group. FIG. 12B. Ex vivo invasiveness of FLS isolated from healthy rats or arthritic rats, treated with vehicle or IbTX. N=5 rats per group. FIG. 12C. Representative images from the joints of arthritic rats, treated with vehicle or IbTX, and stained with Safranin O-fast green (red: cartilage & bone marrow; blue: bone). FIG. 12D. Number of urine spots after rats received a single injection of vehicle, paxilline, or IbTX and were gavaged with 1 ml drinking water. N=6 rats per group. FIG. 12E. Representative tremor measurements in rats 10 min after the injection of vehicle, paxilline, or IbTX. *p<0.05, **p<0.01, ***p<0.001.

FIG. 13. Intestinal permeability of healthy rats and rat with CIA to 4 kDa dextran. The GI barrier is permeable to 4 kDa dextran. Healthy rats □, [symbol] and rats on the day of CIA onset (▪) received a single oral bolus of 60 mg/kg 4 kDa FITC-dextran (filled squares) or saline (open squares). A sample of blood was collected 6 hrs later for plasma preparation, and detection of serum levels of the FITC-dextran was measured at 590 nm using a fluorescent plate reader. Plasma from rats having received saline with no dextran were used as controls (Q). Mean±SEM. N=3 rats per group. *p<0.05, **p<0.01.

FIGS. 14A-14C. Microfocal computed tomography (Micro-CT) validation of the therapeutic effects of LrS235. Rats were injected with vehicle (P6N buffer), synthetic ShK-235 (100 μg/kg) every other day, or orally administered with LrGusA (1×10⁹ CFU) or LrS235 (1×10⁹ CFU) daily for 21 days from the first day of the onset of the clinical symptoms of arthritis. Micro-CT scan was performed to assess bone damage at the end of the experiments. FIG. 14A. 3D reconstructed bones of ankle in different groups. FIG. 14B. Cross-section at the level of metatarsals. FIG. 14C. Pseudocolor of micro-CT data from FIG. 14A. Red color corresponds to low density pixels, indicating area of bone erosion and cartilage damage, blue color corresponds to high density pixels, indicating high density bone.

FIGS. 15A-15C. ShK-235, delivered via injection or LrS235, is not immunogenic. FIG. 15A. ELISA plates were coated with 10 μg/ml of either porcine collagen II, ShK-235, or HsTX1[R14A]. Sera from rats with CIA treated with vehicle (□), ShK-235 (▪), LrGusA (▴), or LrS235 (▾) were tested at 10-fold dilution steps starting from a 1:100 dilution. The serum from non-immunized rats was used as a negative control (●). Each column represents the mean OD of 4 individual rat sera ±SEM. FIG. 15B. Data from different PBMC donors were normalized to the unstimulated cells with 100% CELLTRACE™ violet. The higher dilution of CELLTRACE™ Violet indicates a higher division rate. Data are present as Mean±SEM. FIG. 15C. Representative photos of the ELISA plates from FIG. 4A. ELISA plates were coated with 10 μg/ml of either collagen II, ShK-235, or the Kv1.3 blocker HsTX1[R14A].

FIGS. 16A-16B. Lack of antibodies against ShK-235 in rats after 8 weeks of treatment. ELISA plates were coated with 10 μg/ml of ShK-235 or the Kv1.3 blocker HsTX1[R14A] with no sequence or structure homology to ShK-235, before incubation with serum from the healthy rats treated daily for 8 weeks (FIG. 16A) or treated daily for weeks, untreated for 12, and treated again daily for 1 week (FIG. 16B) with vehicle (◯), LrGusA (▴), or LrS235 (⋄). Serum was diluted 1:100, 1:1000, and 1:100000, and bound IgG were detected using anti-rat IgG-HRP and TMB substrate at 650 nm. N=8 rats per group (4 males, 4 females).

### DETAILED DESCRIPTION

New therapeutics that combine efficacy with limited side effects and can be delivered non-invasively are needed to adequately treat patients with diseases mediated by cells of the immune system and/or fibroblasts or fibroblast-like cells. Many treatments for disorders that mediated by cells of the immune system and/or fibroblasts or fibroblast-like cells rely upon repeated injections of recombinant peptides, antibodies, and other biologics, which exert their effects on immune cells⁵⁰⁻⁵⁶. Potassium channel-expressing CCR7⁻ effector memory T (T_{EM}) lymphocytes are significant players in the pathogenesis of multiple autoimmune diseases and in some aspects, blocking these channels can reduce disease severity in rat models of RA and patients with plaque psoriasis, for example. Repeated injection to block these channels is expensive and increases the risk of pain and infection at the injection site, leading to reduced patient compliance and therefore reduced efficacy^{21,57}. For these reasons, orally bioavailable drugs are preferable. The use of probiotics to deliver biologics, like ShK-235 and iberiotoxin (IbTX), for example, overcomes these challenges such as sensitivity to stomach acid and to the large amounts of proteases in the duodenum, as the bacteria deliver the compound of interest directly into the intestine.

The present disclosure describes methods and compositions for treating or preventing one or more diseases or conditions, such as one or more diseases or conditions that may be mediated by cells of the immune system and/or fibroblasts or fibroblast-like cells. In particular, disclosed herein are microorganisms, for example, bacteria, that can be induced to recombinantly express one or more biologically active peptides in the gastrointestinal tract of a subject to treat diseases or conditions, such as one or more diseases or conditions that may be mediated by cells of the immune system and/or fibroblasts or fibroblast-like cells like, for example, rheumatoid arthritis. The present disclosure also relates to the production of recombinant microorganisms by introducing into the microorganism one or more polynucleotides encoding the one or more biologically active peptides.

The results encompassed herein demonstrate a novel therapeutic strategy for the treatment of diseases and conditions including diseases and conditions that may be mediated by cells of the immune system and/or fibroblasts or fibroblast-like cells, for example, rheumatoid arthritis and other autoimmune diseases, via the oral delivery of a probiotic chassis engineered to secrete one or more biologically active peptides, for example, one or more biologically active peptides including but not limited to potassium channel blockers. The data support the feasibility of an approach in having L. reuteri LJ01 secrete physiologically relevant amounts of functional ShK-235 or IbTX to effectively suppress joint inflammation in a rat model of RA. The data also show that a gut-delivered peptide can be absorbed into the serum as a functional entity, dispelling conventional wisdom that the gut barrier is too sturdy to allow traversal of peptides into systemic circulation and the rest of the body.

In some aspects, engineered microbes for the delivery of biologics treat various conditions, e.g., chronic inflammatory disorders and metabolic diseases. A genetically engineered probiotic delivery system was developed to deliver a small molecular biologic to the intestinal tract with high efficacy and minimized side effects. An inducible system was constructed for expression in the probiotic Lactobacillus reuteri to secret functional potassium blockers (e.g., probiotic Lactobacillus reuteri expressing and secreting ShK-235, IbTX) (e.g., LrS235, LrIbTX). In some aspects, the small molecular biologic expressed by the microorganism is capable of blocking potassium channel currents and preferentially inhibiting human T effector memory (T_{EM}) cells proliferation in vitro. In some aspects, a single oral gavage of healthy rats with, e.g., LrS235 or LrIbTX, results in adequate functional ShK-235 or IbTX, respectively, in the circulation to reduce inflammation in a delayed-type hypersensitivity model of atopic dermatitis mediated by T_{EM} cells. Furthermore, in some aspects, daily oral gavage of e.g., LrS235 or LrIbTX, dramatically reduces clinical signs of disease and joint inflammation in rats with a model of rheumatoid arthritis without eliciting immunogenicity against ShK-235 or IbTX, respectively. Thus, in some aspects, small molecular biologic-expressing probiotic microorganisms, e.g., L. reuteri, can be efficaciously used as a novel oral delivery platform and can provide a efficacious strategy to deliver biologics with great translational potential.

### I. Examples of Definitions

Throughout this application, the terms “about,” “substantially,” and “approximately” are used according to their plain and ordinary meaning in the area of cell and molecular biology to indicate a deviation of ±10% of the value(s) to which they are attached.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that aspects described herein in the context of the term “comprising” may also be implemented in the context of the term “consisting of” or “consisting essentially of.”

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “inhibiting,” “decreasing,” or “reducing” or any variation of these terms includes any measurable decrease (e.g., a 5%, 10%, 15%, 20%, 25%, 30%, 35%, 40%, 45%, 50%, 55%, 60%, 65%, 70%, 75%, 80%, 85%, 90%, 95%, or 99% decrease) or complete inhibition to achieve a desired result. The terms “improve,” “promote,” or “increase” or any variation of these terms includes any measurable increase (e.g., a 5%, 10%, 15%, 20%, 25%, 30%, 35%, 40%, 45%, 50%, 55%, 60%, 65%, 70%, 75%, 80%, 85%, 90%, 95%, or 99% increase) to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

A “nucleic acid,” as used herein, is a molecule comprising nucleic acid components and refers to DNA or RNA molecules. It may be used interchangeably with the term “polynucleotide.” A nucleic acid molecule is a polymer comprising or consisting of nucleotide monomers, which are covalently linked to each other by phosphodiester-bonds of a sugar/phosphate-backbone. Nucleic acids may also encompass modified nucleic acid molecules, such as base-modified, sugar-modified or backbone-modified etc. DNA or RNA molecules. Nucleic acids can exist in a variety of forms such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, saRNA, and complementary sequences of the foregoing described herein.

The term “polynucleotide” refers to a nucleic acid molecule that can be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA, or synthetic), analogs thereof, or a combination thereof. Additional coding or non-coding sequences may, but need not, be present within a polynucleotide.

The terms “protein,” “polypeptide,” or “peptide” are used herein as synonyms and refer to a polymer of amino acid monomers, i.e., a molecule comprising at least two amino acid residues. Polypeptides may include gene products, naturally occurring polypeptides, synthetic polypeptides, homologs, orthologs, paralogs, fragments and other equivalents, variants, and analogs of the foregoing. Polypeptides may be a single molecule or may be a multi-molecular complex such as a dimer, trimer or tetramer. A protein comprises one or more peptides or polypeptides, and may be folded into a 3-dimensional form, which may be required for the protein to exert its biological function.

The term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group). In some aspects, percent identity of two sequences is determined using the BLASTN or BLASTP algorithm, as available on the United States National Center for Biotechnology Information (NCBI) website.

Percentage identity is obtained by determining the number of identical positions at which the sequences to be compared correspond, dividing this number by the number of positions compared (e.g., the number of positions in the reference sequence) and multiplying this result by 100.

In some aspects, the degree of similarity or identity is given for a region that is at least, at most, exactly, or between any two of about 50%, about 60%, about 70%, about 80%, about 90%, or about 100% of the entire length of the reference sequence. For example, if the reference nucleic acid sequence consists of 200 nucleotides, the degree of identity is given for at least, at most, exactly, or between any two of about 10, about 20, about 30, about 40, about 50, about 60, about 70, about 80, about 90, about 100, about 120, about 140, about 160, about 180, or about 200 nucleotides, in some aspects, continuous nucleotides. In some aspects, the degree of similarity or identity is given for the entire length of the reference sequence.

“Individual, “subject,” and “patient” are used interchangeably and can refer to either a human or non-human, such as primates, mammals, and vertebrates. In particular aspects, the subject is a human. The subject is of any age, gender, or race. The subject can be a patient, e.g., have or be suspected of having a disease (that may be referred to as a disorder or medical condition), such as one or more immune system-mediated disorders. The subject may be undergoing or have undergone treatment. The subject may be asymptomatic. The subject may be a healthy individual desirous of prevention of a disease or condition.

As will be understood from context, “risk” of a disease, disorder, and/or condition refers to a likelihood that a particular individual will develop the disease, disorder, and/or condition. In some embodiments, risk is expressed as a percentage. In some embodiments, risk is, is at least or is at most from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes. An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

As used herein, “treat,” “treating,” or “treatment” or equivalent terminology refer to both therapeutic treatment and prophylactic or preventative measures, wherein the object is to prevent or slow down (lessen) an undesired physiological change or disorder, such as the growth, development, or spread of one or more symptoms or manifestation of a disease or condition. As an example, the disease or condition may be mediated by cells of the immune system and/or fibroblasts or fibroblast-like cells, and the one or more symptoms may be, for example, inflammation. For purposes of this disclosure, beneficial or desired clinical results include, but are not limited to, alleviation or amelioration of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” does not necessarily indicate complete eradication or cure of the disease or condition, or associated symptoms thereof. Those in need of treatment include those already with the condition or disorder as well as those prone to have the condition or disorder or those in which the condition or disorder is to be prevented. The results of treatment can be determined by methods known in the art, such as determination of reduction of, e.g., tumor burden or viral load, determination of restoration of function, or other methods known in the art.

As used herein, “prevent,” and similar words such as “prevented,” “preventing,” etc., indicate an approach for preventing, inhibiting, or reducing the likelihood of the occurrence or recurrence of, a disease or condition, e.g., an immune system-mediated disorder. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition. Prevention may be considered complete when onset of a disease, disorder, or condition has been delayed for a predefined period of time.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of an immune system-mediated disorder may include but is not limited to a reduction in pain, edema, elevated temperature, and/or inflammation or prevention of immune rejection. Treatment of an immune system-mediated disorder may also refer to prolonging survival of a subject with an immune system-mediated disorder.

The term “therapeutically effective amount” refers to an amount sufficient to produce a desired therapeutic result, for example an amount of a recombinant organism expressing one or more biologically active peptides that are heterologous to the microorganism or a composition comprising such a recombinant organism sufficient to improve at least one symptom of a medical condition in a subject to whom the recombinant organism or composition are administered.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

### II. Therapeutically-Relevant Ion Channels

Disclosed herein, in some aspects, are methods of treating or preventing one or more diseases or conditions, including at least one or more disorders that may be mediated by cells of the immune system and/or fibroblasts or fibroblast-like cells. In some aspects, the one or more diseases or conditions are characterized by at least one symptom, and the at least one symptom is inflammation.

In some aspects, the inflammation is caused at least in part by the proliferation and activation of immune cells. The pathogenesis role of T lymphocytes in inflammatory disorders that may be mediated by cells of the immune system and/or fibroblasts or fibroblast-like cells has been extensively studied. Whereas CCR7⁺ naïve and central memory T (T_{CM}) cells are the predominant T lymphocyte populations in the circulation and lymphoid organs, most T cells in the synovium and synovial fluid of patients with inflammatory disorders are CCR7⁻ effector memory T (T_{EM}) cells, making them a desirable therapeutic target in some aspects. Thus, in some aspects, the inflammation is caused at least in part by the proliferation and activation of CCR7⁻ effector memory T-cells (T_{EM} cells). In particular aspects, one or more biologically active peptides produced and secreted by a recombinant microorganism inhibits the proliferation and/or activation of CCR7⁻ T_{EM} cells, thereby reducing inflammation caused by T_{EM} cells, for example. Thus, in some aspects, targeting T_{EM} cells can reduce inflammation and/or treat or prevent one or more disorders that may be mediated by cells of the immune system.

In some aspects, the inflammation is caused at least in part by the proliferation and activation of class-switched B-cells. In particular aspects, one or more biologically active peptides produced and secreted by a recombinant microorganism inhibits the proliferation and/or activation of class-switched B-cells, thereby reducing inflammation caused by class-switched B-cells, for example. Thus, in some aspects, targeting class-switched B-cells can reduce inflammation and/or treat or prevent one or more disorders that may be mediated by cells of the immune system.

In some aspects, the inflammation is caused at least in part by the proliferation and activation of fibroblasts and/or fibroblast-like cells. In some aspects, the fibroblast-like cells are fibroblast-like synoviocytes (FLS). In some aspects, the inflammation is caused at least in part by the proliferation and activation of FLS. In particular aspects, one or more biologically active peptides produced and secreted by a recombinant microorganism inhibits the proliferation, invasiveness, and secretion of cytokines, chemokines, proteases and growth factors by FLS without inducing cell death, thereby reducing inflammation caused by FLS, for example. Thus, in some aspects, targeting fibroblasts or fibroblast-like cells like FLS can reduce inflammation and/or treat or prevent one or more disorders that may be mediated by fibroblasts or fibroblast-like cells.

Human and rat T-cells and B-cells express low levels of two K⁺ channels, Kv1.3 and KCa3.1, that regulate plasma membrane potential and thus influx of Ca²⁺, a crucial second messenger in T-cell activation²⁻⁵. Upon activation by, for example, an autoantigen, CCR7⁻ effector memory T (T_{EM}) cells upregulate Kv1.3 whereas CCR7⁺ naïve and central memory T (T_{CM}) cells upregulate KCa3.1 and escape Kv1.3 blockers^{2,6-9}. Similarly, upon activation by, for example, an autoantigen, IgD⁻ B-cells (class-switched memory) upregulate Kv1.3 whereas IgD⁺ B-cells (naïve and early memory) upregulate KCa3.1.

Thus, CCR7⁻ T_{EM} cells and IgD⁻ class-switched memory B-cells are exquisitely sensitive to Kv1.3 blockers, which selectively inhibit T_{EM} cells and IgD⁻ class-switched memory B-cells, whereas CCR7⁺ naïve T and T_{CM} cells and IgD⁺ naïve and early memory B-cells rely on KCa3.1 and are insensitive to Kv1.3 blockers. Additionally, CCR7⁻ T_{EM} cells and IgD⁻ class-switched memory B-cells are involved in the pathophysiology of many autoimmune diseases upon activation of the cells by an autoantigen^{2-5,8-17}.

The human genome encodes more than 80 K⁺ channel subunits. The diversity in K⁺ channels is further increased by the ability of subunits to form functional homotetramers or heterotetramers within a family²⁸. In T lymphocytes, K⁺ channels are crucial in regulating signaling downstream from the T-cell receptor, and thus T-cell activation²⁻⁴. During T-cell activation, a large Ca²⁺ influx through the Ca²⁺ release-activated Ca²⁺ channel (CRAC; composed of Orai subunits and stromal interacting molecule 1 (STIM1) proteins) is necessary for T-cell activation²⁹. Following T-cell receptor engagement and endoplasmic reticulum Ca²⁺ store depletion, Ca²⁺ unbinds from STIM1's low-affinity luminal EF hand domain. This triggers STIM1 first to oligomerize and then to translocate to the plasma membrane where it activates Orai1 to form a Ca²⁺-selective pore. As Ca²⁺ enters the cell, it changes the plasma membrane potential and reduces the driving force needed for Ca²⁺ influx. K⁺ channels are physically associated with the T-cell receptor complex and thus localize to the immune synapse; they open following Ca²⁺ influx, leading to a K⁺ efflux and restoration of resting membrane potential, thus sustaining the Ca²⁺ influx needed for activations^{8,30-32}. Blocking K⁺ channels blunts the Ca²⁺ influx, preventing the activation of Ca²⁺-dependent transcription factors and resulting in an inhibition of T-cell cytokine secretion, proliferation, and motility^{8,17,33,34}.

Kv1.3^{high} T_{EM} cells play a major role in the pathogenesis of many autoimmune and inflammatory diseases, including multiple sclerosis, psoriasis, glomerulonephritis, ulcerative colitis, asthma, or type 1 diabetes⁸⁻¹⁷. Kv1.3 blockers including ShK analogs have also proven effective in reducing clinical sign severity in animal models of these diseases and in patients with active plaque psoriasis^{6,8,16,17,33,47}-49.

The Kv1.3 channel, like about half of all K⁺ channels, is intrinsically voltage dependent; its conformation changes when the cell is depolarized, leading to an open channel that is specific to K⁺ ions. The pore for specific conduction of K⁺ ions is right in the middle of subunits pre-assembled as a tetramer. A single resting T-cell obtained from human peripheral blood typically has approximately 400 functional Kv1.3 channels in the plasma membrane. In T-cells under basal conditions, the resting potential is maintained at about −50 mV by only a small fraction of the available Kv1.3 channels. In essence, Kv1.3 by its sigmoid voltage dependence provides protection against depolarization of the membrane potential, thereby ensuring that the lymphocyte's membrane potential does not become significantly depolarized, even if Ca²⁺ is entering the cell.

Kv1.3 is found in the plasma membrane of T-cells as part of a signaling complex that includes β1-integrin, a PDZ-domain protein called hDlg (or SAP97), an auxiliary channel subunit Kvβ2, an adapter protein ZIP (or sequestosome 1/p62), and p56lck (Lck). It is also present in the inner membrane of mitochondria, where it has been reported to be the target for the apoptotic BAX protein, which binds and occludes the channel pore, thereby altering the membrane potential of the mitochondrion.

A second type of K⁺ current in T-cells is activated by a rise in cytosolic Ca²⁺, rather than by changes in membrane potential. In human T and B cells, the Ca²⁺-activated K⁺ current is characterized by an intermediate single channel conductance that uniquely distinguishes it from small- or large-conductance Ca²⁺-activated K⁺ channels. The channel is closed under resting conditions with low basal cytosolic Ca²⁺ and opens rapidly if Ca²⁺ rises, with an effective binding coefficient of approximately 300 nM and a high degree of cooperativity consistent with a tetrameric channel. The essential pore-forming subunit is KCa3.1, with a six-transmembrane segment architecture and a tetrameric pore similar to Kv1.3 and other voltage-gated K⁺ channels. CaM bound to the C-terminus of the KCa3.1 subunit functions as an essential Ca²⁺-sensing subunit to activate the channel rapidly upon Ca²⁺ binding. Thus, during TCR-evoked Ca²⁺ signaling, the opening of KCa3.1 channels contributes a K⁺ current that makes the membrane potential more negative (hyperpolarized).

T-cell Ca²⁺ release-activated Ca²⁺ (CRAC) currents corresponding to CRAC channels are highly Ca²⁺-selective type of ‘store-operated’ Ca²⁺ entry channels (permeability ratios >1000 for Ca²⁺ over monovalent cations). Activating stimuli of these channels is depletion of ER luminal Ca²⁺, rather than a rise in cytosolic Ca²⁺. When the ER Ca²⁺ store is depleted, Stim proteins physically convey the signal from the ER to the plasma membrane to activate CRAC channels (70, 73, reviewed in 74). The N-terminus of Stim residing within the ER lumen contains a low-affinity EF-hand domain that binds Ca²⁺ when the Ca²⁺ store is filled. If the ER store is depleted, Ca²⁺ ions unbind from Stim, and Stim proteins migrate toward and accumulate in puncta next to the plasma membrane. Once it is immediately adjacent to the plasma membrane, Stim triggers CRAC channels formed by Orai subunits to open, allowing Ca²⁺ to enter the cell. When expressed alone, Orai is seen by single-molecule photobleaching as a dimer, but when expressed as a conducting channel activated by the C-terminus of Stim, Orai was shown to be tetrameric, suggesting that Stim coordinates assembly of stable but inactive Orai dimers into functional tetramers. In T-cells, CRAC channels triggered by STIM1 and formed by Orai1 are required for Ca²⁺ entry, based upon RNA knockdown of STIM1 and Orai1 in human T-cell lines and in human T-cells, and from nearly complete inhibition of thapsigargin- or TCR-induced Ca²⁺ entry by dominant-negative constructs of Orai1 in human T-cells.

Intermediate conductance KCa3.1 channels are functionally related to small conductance KCa channels (KCa 2.1, 2.2, and 2.3). Small conductance channels are sensitive to block by apamin (100 pM-10 nM), which distinguishes them from all other KCa channels. The group additionally is made up of Intermediate conductance channels (KCa3.1 (IK)). Both intermediate conductance and small conductance channels play roles in processes involving calcium-dependent signaling in both electrically excitable and non-excitable cells. If they do not bind calcium directly, they detect it via calmodulin, which is constitutively bound to the C-terminal region. Binding of calcium to calmodulin results in conformational changes that are in turn responsible for channel gating.

A second group of Ca²⁺ activated K⁺ channels include large conductance KCa channels (KCa1.1, also known as BK channel, MaxiK, Slo, or Slo1, and encoded by KCNMA1. These channels are large conductance, voltage and calcium-sensitive potassium channels. The channels are activated by both membrane depolarization or increase in cytosolic Ca²⁺ that mediates export of K⁺. It is also activated by the concentration of cytosolic Mg²⁺. The channels can be formed by 2 subunits: the pore-forming alpha subunit, which is the product of this gene, and the modulatory beta subunit. Intracellular calcium regulates the physical association between the alpha and beta subunits. The channels bind calcium independently of calmodulin, but binding is mediated by at least three divalent cation binding sites in the cytoplasmic carboxyl domain of each channel subunit. The KCa1.1 potassium channel contributes to repolarization of the membrane potential. Other members of this group are KCa4.1 (Slack or Slo2.2), KCa4.2 (Slick or Slo2.1), and KCa5.1 (Slo3).

The KCa1.1 K⁺ channel is the predominant K⁺ channel at the plasma membrane of human and rat fibroblast-like synoviocytes (FLS). FLS form the lining of the synovium and secrete lubricants into the joints under physiologic conditions. In adult rheumatoid arthritis (RA), FLS are activated and acquire a “tumor-like” phenotype. This is characterized by loss of contact inhibition, secretion of pro-inflammatory and proangiogenic cytokines, chemokines and growth factors, secretion of proteases, and invasiveness. Potassium (K⁺) channels are often upregulated by metastatic tumor cells and other highly migratory and aggressive cells, and the KCa1.1 K⁺ channel is a major regulator of pathogenic features of RA-FLS. Blocking KCa1.1 expression or function inhibits proliferation, invasiveness, and secretion of cytokines, chemokines, proteases and growth factors by FLS without inducing cell death (see, e.g., C. Beeton, Expert Opin Ther Targets, 2017 December; 21(12): 1077-1081, incorporated by reference herein in its entirety).

### III. Biologically Active Peptides

Disclosed herein, in some aspects, are methods of treating or preventing a disease or condition, such as a disease or condition including but not limited to a disease or condition mediated by cells of the immune system and/or fibroblasts or fibroblast-like cells, by administering one or more biologically active peptides produced and secreted by a recombinant microorganism.

In some aspects, the one or more biologically active peptides are administered to treat or prevent a disease or condition including but not limited to a disease or condition mediated by cells of the immune system and/or fibroblasts or fibroblast-like cells. In some aspects, treating or preventing an immune system disorder comprises administration of the one or more biologically active peptides to inhibit the proliferation and/or activation of immune cells, such as CCR7⁻ T_{EM} cells and/or IgD⁻ class-switched memory B-cells. In some aspects, the one or more biologically active peptides produced and secreted by a recombinant microorganism to inhibit the proliferation and/or activation of immune cells, such as CCR7⁻ T_{EM} cells and/or IgD⁻ class-switched memory B-cells, are immunomodulatory peptides.

In some aspects, treating or preventing a disease or condition including but not limited to a disease or condition mediated by cells of the immune system and/or fibroblasts or fibroblast-like cells comprises administration of the one or more biologically active peptides to inhibit the proliferation and/or activation of fibroblasts and/or fibroblast-like cells, such as fibroblast-like synoviocytes (FLS). In some aspects, the one or more biologically active peptides produced and secreted by a recombinant microorganism to inhibit the proliferation and/or activation of fibroblasts and/or FLS are non-immunomodulatory peptides.

In some aspects, treating or preventing a disease or condition mediated by cells of the immune system and/or fibroblasts or fibroblast-like cells comprises administration of the one or more biologically active peptides to inhibit the proliferation and/or activation of both immune cells and fibroblasts and/or fibroblast-like cells.

The one or more biologically active peptides can also be administered to treat or prevent a disease or condition in addition to or other than a disease or condition mediated by cells of the immune system and/or fibroblasts or fibroblast-like cells.

### A. Ion Channel Blockers

In some aspects, the biologically active peptides are immunomodulatory peptides, and the immunomodulatory peptides are immune cell ion channel blockers. In some aspects, the proliferation and/or activation of immune cells, such as CCR7⁻ T_{EM} cells and/or IgD⁻ class-switched memory B-cells, is inhibited by blocking ion channels present in the plasma membrane of the immune cells. In some aspects, the ion channels comprise potassium channels. In some aspects, the potassium channels comprise Kv1.3 potassium channels, and, in some aspects, inhibiting T_{EM} cells by blocking Kv1.3 potassium channels with one or more potassium channel blockers can treat or prevent one or more diseases or conditions including one or more diseases or conditions mediated by cells of the immune system.

In some aspects, the biologically active peptides block ion channels in cells in addition to or other than immune cells. In some aspects, the ion channels blocked by the biologically active peptides comprise ion channels in fibroblasts and/or fibroblast like cells. In some aspects, the fibroblast-like cells comprise FLS. In some aspects, the proliferation and/or activation of fibroblasts and/or fibroblast like cells, such as FLS, is inhibited by blocking ion channels present in the plasma membrane of the cells. In some aspects, the ion channels comprise potassium channels. In some aspects, the potassium channels comprise KCa1.1 potassium channels, and, in some aspects, inhibiting fibroblasts and/or fibroblast like cells, such as FLS, by blocking KCa1.1 potassium channels with one or more potassium channel blockers can treat or prevent one or more diseases or conditions including one or more diseases or conditions mediated by fibroblasts or fibroblast-like cells.

T-cell and B-cell potassium channels such as Kv1.3 can be blocked by a number of pharmacological agents. In some aspects, Kv1.3 is blocked by small organic compounds in the millimolar to nanomolar range or peptide toxins in the nanomolar to picomolar range of potency. In some aspects, Kv1.3 blockers include but are not limited to canonical but low potency agents previously known to block ‘delayed rectifier’ K⁺ channels in nerves, ionic blockers, Ca²⁺ antagonists, and calmodulin (CaM) antagonists, scorpion peptides, sea anemone peptides, compounds discovered as a result of high-throughput screening efforts, progesterone, and non-peptidyl compounds resulting from a screen of compounds isolated from the shrub plant Ruta graveolens and the carrot family plant Ammi visnaga.

A variety of small organic inhibitors block Kv1.3 by gaining access to the inner vestibule of the channel, and several of these lipophilic compounds stabilize the inactivated state of the channel. The most potent non-peptidyl inhibitor of Kv1.3 is PAP-1 with an IC₅₀ value of 2 nM. In specific aspects, the inhibitor of Kv1.3 is PAP-1.

For the scorpion and sea anemone peptide toxins, alanine scanning and complementary mutagenesis have revealed the binding interactions with Kv1.3. These toxins block from the outside of the cell. A critical positively charged lysine residue on the toxin partially enters the pore between subunits of the tetrameric channel, occupying a site where K⁺ ions normally bind. Several scorpion, sea anemone, and other peptide toxin variants with improved selectivity for Kv1.3 have been engineered and in some aspects include but are not limited to the following peptides, as well as derivatives or analogs thereof: OSK-1-Lys16Asp20, which blocks with an IC₅₀ value of 3 pM and exhibits >300-fold selectivity for Kv1.3 over closely related K⁺ channels; BmK1; BmKTX-Arg11Thr28His33 peptide inhibitor (ADWX-1), which blocks Kv1.3 with an IC₅₀ value of 1 pM; Vm24; HsTX1 and its analogs, including but not limited to HsTX1[R14A]; Osk1; kaliotoxin; charybdotoxin; margatoxin; AcK1; Agitoxin-2; EgK5; VvK1; anuroctoxin; and ShK.

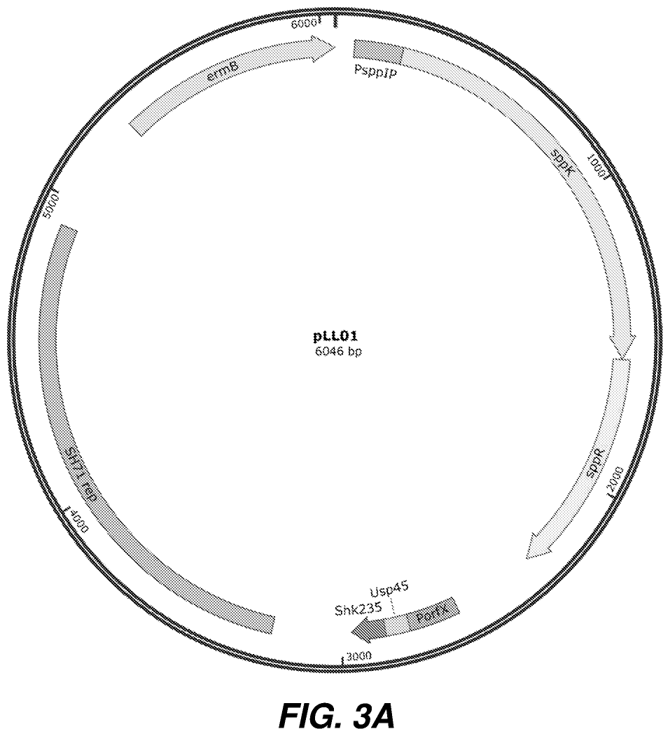

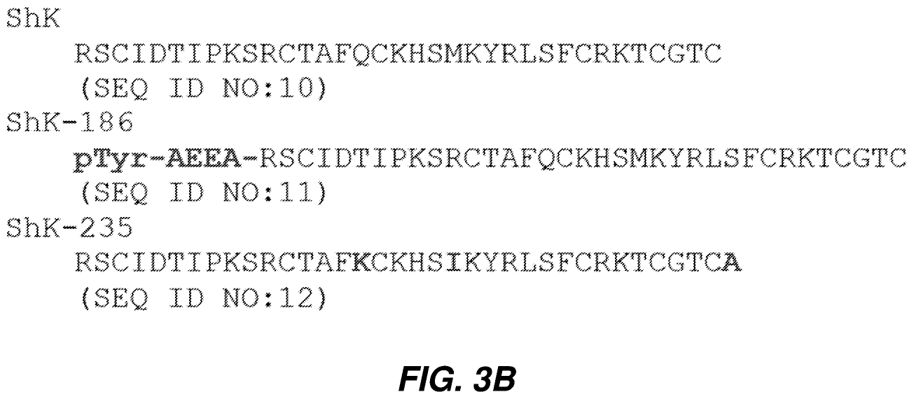

ShK is a 35-amino acid peptide with a molecular mass of 4055 Da having the amino acid sequence RSCIDTIPKSRCTAFQCKHSMKYRLSFCRKTCGTC (SEQ ID NO:10; FIG. 3B), and it contains 6 cysteine residues located at positions 3, 12, 17, 28, 32, and 35. Its three disulfide bonds were identified as Cys3 to Cys35, Cys12 to Cys28, and Cys17 to Cys32. ShK-186 (a.k.a. dalazatide) is derivative of ShK having 1-phosphotyrosine attached to the α-amino group of ShK-Arg1 through a 20 Å hydrophilic 2-aminoethoxy-2-ethoxy acetic acid (AEEA; mini-PEG) linker and having an amidated C-terminal Cys. ShK-186 therefore has the amino acid sequence pTyr-AEEA-RSCIDTIPKSRCTAFQCKHSMKYRLSFCRKTCGTC (SEQ ID NO:11; FIG. 3B). ShK-186 is a potent and selective blocker of Kv1.3 that has been extensively tested in rats, non-human primates, and in Phase 1A and 1B clinical trials in healthy volunteers and patients with a T_{EM} cell-mediated autoimmune disease^{2-4,16,18}.

Because ShK-186 cannot be produced recombinantly due to the presence of a non-amino acid adduct in the peptide, the derivative ShK-235 has been generated that retains the inhibitory function and selectivity for inhibition of Kv1.3 channels found in ShK-186 and can be produced recombinantly in bacteria. ShK-235 has the amino acid sequence RSCIDTIPKSRCTAFKCKHSIKYRLSFCRKTCGTCA (SEQ ID NO:12; FIG. 3B). Extensive literature has been published showing the selectivity of ShK-186 and ShK-235 for Kv1.3 over other ion channels, the preferential inhibition of CCR7⁻ T_{EM} cells over CCR7⁺ naïve and T_{CM} cells by Kv1.3 blockers, and the efficacy of Kv1.3-selective ShK analogs in reducing disease severity in rat models of autoimmune diseases, including RA^{2-5,19,73}. In some aspects, the Kv1.3 blocker comprises a peptide having an amino acid sequence that has, has at least, or has at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:12. In some aspects, the Kv1.3 blocker comprises a peptide having an amino acid sequence comprising SEQ ID NO:12. In some aspects, the Kv1.3 blocker comprises a peptide having an amino acid sequence consisting of SEQ ID NO:12. In specific aspects, the Kv1.3 blocker is ShK-235. In some embodiments, the ShK-235 is synthetic. In other embodiments, the ShK-235 is produced and secreted by a recombinant microorganism (e.g., bacteria) (e.g., L. reuteri).

A variety of non-peptidyl compounds and peptides inhibit KCa3.1 channels. In some aspects, these include but are not limited to the following compounds and peptides, as well as derivatives or analogs thereof: clotrimazole and the potent and selective analogs TRAM-34 and ICA-17043, charybdotoxin and its analog ChTX-E32, and maurotoxin.

Ion channel modulators that block the KCa1.1 channel include but are not limited to, in some aspects, the following compounds: iberiotoxin (IbTX); slotoxin; limbatustoxin; Lq2; martentoxin; and derivatives or analogs thereof. In specific aspects, the KCa1.1 blocker is IbTX. In some embodiments, the IbTX is synthetic. In other embodiments, the IbTX is produced and secreted by a recombinant microorganism (e.g., bacteria).

### B. Other Relevant Peptides

In some aspects, the biologically active peptides are peptides other than ion channel blockers. Other relevant toxins and peptides include but are not limited to, in some aspects, the following toxins and peptides, as well as derivatives or analogs thereof: exendin-4; insulin and proinsulin; liraglutide and pramlintide; glatiramer; enfuvirtide; buserelin and leuprorelin; teduglutide; and thymalfasin. In some aspects, one or more biologically active peptides other than ion channel blockers produced and secreted by a recombinant microorganism are administered to treat or prevent a disease or condition in addition to or other than a disease or condition mediated by cells of the immune system and/or fibroblasts or fibroblast-like cells. In some aspects, the disease or condition comprises type 2 diabetes, HIV, cancer, short bowel syndrome, or hepatitis (including hepatitis B and C).

### IV. Recombinant Microorganisms & Compositions Thereof

Disclosed herein, in some aspects, are methods of treating or preventing one or more diseases or conditions, such as one or more diseases or conditions mediated by cells of the immune system and/or fibroblasts or fibroblast-like cells, by administering one or more biologically active peptides produced and secreted by a recombinant microorganism. The microorganism according to the invention can be any microorganism, including bacteria, yeasts or fungi, suitable for mucosal delivery. In some aspects, the microorganism is a non-commensal, non-colonizing microorganism. In some aspects, the microorganism is a non-pathogenic microorganism. In some aspects, the microorganism is a probiotic microorganism. In some aspects, the microorganism is a gram-positive bacterium.

In specific aspects, the microorganism is a probiotic microorganism. Probiotic organisms are known to the person skilled in the art. Probiotic organisms include, but are not limited to, bacteria such as Lactobacillus sp., Lactococcus sp. and yeasts such as Saccharomyces cerevisiae, for example, S. cerevisiae subspecies boulardii. In some aspects, the microorganism is a lactic acid bacterium chosen from the group consisting of Lactobacillus, Leuconostoc, Pediococcus, Lactococcus, Streptococcus, Aerococcus, Carnobacterium, Enterococcus, Oenococcus, Teragenococcus, Vagococcus, and Weisella.

In specific aspects, the microorganism is from a Lactobacillus species strain. In specific aspects, the microorganism is a Lactobacillus reuteri strain. In specific aspects, the microorganism is Lactobacillus reuteri ATCC PTA 6475 or a derivative thereof. In specific aspects, the microorganism is LJ01. Lactobacillus reuteri LJ01 is a derivative of a well-characterized commercial probiotic strain that survives transit through the gastrointestinal tract without long-term colonization and that displays anti-inflammatory and bone-protective properties in itself. Thus, in some aspects, L. reuteri LJ01 can produce and secrete one or more biologically active peptides for the treatment or prevention of one or more diseases or conditions, such as one or more diseases or conditions including but not limited to one or more diseases or conditions mediated by cells of the immune system and/or fibroblasts or fibroblast-like cells.

In specific aspects, one or more biologically and functionally active peptides, for example, ShK-235 and/or IbTX, are constitutively expressed and secreted by L. reuteri LJ01. In some aspects, the biologically and functionally active peptide, for example, ShK-235 or IbTX, constitutively expressed and secreted by a recombinant microorganism, for example, L. reuteri LJ01, results in the selective block of one or more potassium channels upregulated by activated immune cells, for example, Kv1.3, or expressed by activated fibroblasts or fibroblast-like cells (e.g., FLS), for example KCa1.1; the inhibition of immune cell and/or fibroblast or fibroblast-like cell proliferation; and/or a reduction of clinical severity of one or more diseases or conditions including but not limited to one or more diseases or conditions mediated by cells of the immune system and/or fibroblasts or fibroblast-like cells. In some aspects, delivery of the biologically and functionally active peptide, for example, ShK-235 or IbTX, to the gut by a recombinant microorganism, for example, L. reuteri LJ01, leads to secretion of the peptide in situ in the gut and systemic circulation of active peptide in the serum.

Thus, in some aspects, functionally active ShK-235 is constitutively expressed and secreted by L. reuteri to selectively block Kv1.3 potassium channels upregulated by activated CCR7⁻ T_{EM} cells and or IgD⁻ class-switched memory B-cells, resulting in the inhibition of human T_{EM} cell and or IgD⁻ class-switched memory B-cell proliferation in vitro and a reduction of clinical severity of one or more diseases or conditions, including but not limited to one or more diseases or conditions mediated by cells of the immune system and/or fibroblasts or fibroblast-like cells. In some aspects, delivery of ShK-235 to the gut by L. reuteri LJ01 leads to systemic circulation of active peptide in the serum.

In some aspects, functionally active IbTX is constitutively expressed and secreted by L. reuteri to selectively block KCa1.1 potassium channels upregulated by activated FLS, resulting in the inhibition of human FLS cell proliferation in vitro and a reduction of clinical severity of one or more diseases or conditions, including but not limited to one or more diseases or conditions mediated by cells of the immune system and/or fibroblasts or fibroblast-like cells. In some aspects, delivery of IbTX to the gut by L. reuteri LJ01 leads to systemic circulation of active peptide in the serum.

Aspects of the disclosure concern compositions comprising a therapeutically effective amount a recombinant microorganism expressing one or more biologically active peptides that are heterologous to the microorganism for treatment or prevention of one more diseases or conditions including but not limited to one or more diseases or conditions mediated by cells of the immune system and/or fibroblasts or fibroblast-like cells. In some aspects, the disease or condition is characterized by inflammation as at least one symptom.

In particular aspects a subject that has a disease or condition or that is at risk for having a disease or condition is provided an effective amount of a composition comprising a therapeutically effective amount a recombinant microorganism expressing one or more biologically active peptides that are heterologous to the microorganism. In some aspects, the one or more biologically active peptides comprise an immunomodulatory peptide. In some aspects, the one or more biologically active peptides comprise a non-immunomodulatory peptide. In particular aspects a subject that has a disease or condition mediated by cells of the immune system and/or fibroblasts or fibroblast-like cells or that is at risk for having a disease or condition mediated by cells of the immune system and/or fibroblasts or fibroblast-like cells is provided an effective amount of a composition comprising a therapeutically effective amount a recombinant microorganism expressing one or more biologically active peptides that are heterologous to the microorganism. The compositions may comprise a recombinant microorganism that produces and secretes any one or more biologically active peptides and/or derivatives thereof associated with efficacious therapy to treat or prevent one or more diseases or conditions and/or to enhance therapy to treat or prevent one or more diseases or conditions, including but not limited to one or more diseases or conditions mediated by cells of the immune system and/or fibroblasts or fibroblast-like cells.

### A. Recombinant Microorganisms

In some aspects, the recombinant microorganism comprises a Lactobacillus microorganism, such as L. reuteri. Any strain of L. reuteri may be utilized. Examples of strains of L. reuteri include at least ATCC PTA 6475 (MM4-1A), ATCC 6475::pocR, ATCC PTA 4659 (MM2-3), ATCC PTA 5289 (FJ1), CRL1324, 1048, 1063, 1073, 173.5, 27.4, atcc55739, atcc53608, jw2015, jw2019, ks6, lem83, lr85573, tmw11294, 10c2, p97, pg3b, 32, 676, cp447, 6s15, cp415, lp167.67, lpa1, tmw1146, tmw1137, 100.93, ad23, dbc2, 100-23, dsm20053, ilc4, mlc3, mouse56, n2j, r2lc, 11283, 11284, ke1, ky21, mf14c, mf2-3, mf23, t1, 1204, 1366, atcc55730 (SD2112), cf48-3a, cf48-3a1, DSM 17938::pocR, dsm17938, m27u15, m45r2, m81r43, MM34-4a, mm344a, MM36-1a, mm361a, MV36-2a, mv362a, MV4-1a, mv41a, nck1556, t2, t3, csa9, csf8, hw8, hwb7, d15, hwh3, jcm1081, jcm 1112, lb54, lk146, lk150, lk20, lk94, nck983, nck985, tu160, tu174, 1013, 104r, 20.2, 3c6, 4s17, cp395, 1461, cf4-6g, cf6-2a, DSM20016, dsm20016t, fj1, mm3-1a, mm41a, cf2a0, me261, sr11, uga29, 2010, 6799jm, 6799jm1, lacto6798jm1, lpuph1, bmc1, bmc2, ml1, cr, oneone, dsm20056, fua3043, fua3048, jw2016, l16001, lpupjm1, l16041, n2d, n4i, number20, mouse2, r13, rat19, l722, lms11.1, lms11.3, lr4020, tmw1.1297, 13S14, JW2017, 1068, 63/1, 146/2, 173/3, 173/4, P26, 1704, N5D:1, me262, Mouse 41, Mouse 81, DBC3, ILC3, MHLC1A, MLC1B, MLC4, CS9, CSB7, LK159, LK75, LK139, NCK984, SD2112, MV14-1a, M1F7-J, L3B, 3S3, MM2-3, CF2-7F, CF15-6, SR14, FUA3041, FUA3044, JCM5869, 6798 cm-1, BMC3, L6799, L6800jm-1, Lacto1662, Rat 8, Rat 17, Mouse 20, Mouse 76, uga44-1, other strains listed and described in Frese et al., 2011; Spinler et al., 2014; Oh et al., 2010, and combinations thereof. In some aspects, the strains of L. reuteri include at least ATCC PTA 6475, DSM17938, ATCC PTA4659, ATCC PTA 5289, ATCC 55730, CRL1324, DSM20016, CF4-6G, ATCC55730, CF48-3A1, M27U15, or a combination thereof. In specific aspects, the strain of L. reuteri includes at least ATCC PTA 6475. In specific aspects, the strain of L. reuteri includes at least ATCC PTA 6475 LJ01.

The L. reuteri may be obtained from any suitable source, including commercially, from a research laboratory, as a gift, or isolated from nature. Isolation methods for L. reuteri are known in the art, including at least those described in U.S. Pat. Nos. 5,439,678 and 5,849,289 (both incorporated by reference herein in their entirety), for example.

In specific aspects, L. reuteri is not delivered as a live microorganism, but instead a specific extract or fraction produced from the live microorganism that may be equally bioactive is delivered. This has been demonstrated, for example, in the case of wound healing in animals, in which consumed sonicated mixtures or lysates prepared from live or dead L. reuteri are biologically active in a wound healing model (Poutahidis et al., 2013; Varian et al., 2016). The mixtures, lysates, or extracts may be delivered in pill form or any other standard delivery mode (food or liquid beverages, yogurt, dry powder mixed in water or beverage) in which such mixtures can be effectively dosed and consumed in a biologically active format.

### B. Generation of Recombinant Microorganisms

In some aspects, the recombinant microorganism delivers the one or more biologically active peptides at an intended site, for example, the gastrointestinal tract. The recombinant microorganism expresses the one or more biologically active peptides, after which the one or more biologically active peptides are secreted by the recombinant microorganism. In some aspects, the recombinant microorganism is generated by introducing into a microorganism one or more polynucleotides encoding one or more biologically active peptides. In some aspects, the one or more polynucleotides encoding the one or more biologically active peptides are under the control of one or more regulatory sequences. In some aspects, the one or more regulatory sequences comprise one or more promoter sequences, enhancer sequences, and/or terminator fragments.

In some aspects, the one or more polynucleotides encoding the one or more biologically active peptides comprise a secretory signal sequence (also interchangeably referred to herein as signal peptide or secretion peptide) upstream of the one or more biologically active peptides. In some aspects, the secretory signal sequence comprises a usp45 secretion peptide derived from the L. lactis subsp. cremoris MG1363 ubiquitin specific peptidase 45 and having the following sequence:

(SEQ ID NO: 1)
MEKKIISAILMSTVILSAAAPLSGVYAAA.

In some aspects, the secretory signal sequence comprises a usp45 secretion peptide derived from the L. lactis subsp. cremoris MG1363 ubiquitin specific peptidase 45 and having the following sequence:

MKKKIISAILMSTVILSAAAPLSGVYA (SEQ ID NO:2), corresponding to NCBI accession number ABY84357.1.

In some aspects, the secretory signal sequence comprises a secretion peptide derived from L. reuteri 6475 having a YSIRK gram-positive signal peptide and having the following sequence:

MKKKIISAILMSTVILSAAAPLSGVYA (SEQ ID NO:3), corresponding to NCBI accession number WP_019253893.1.

In some aspects, the secretory signal sequence comprises a secretion peptide derived from a L. plantarum WCFS1 transglycosylase and having the following sequence:

MKKFNFKTMLLLVLASCVFGVVVNVTTSLGPQTAITAQAS (SEQ ID NO:4), corresponding to NCBI accession number WP_011102021.1.

In some aspects, the secretory signal sequence comprises a secretion peptide derived from the putative L. reuteri 6475 N-acetylmuramoyl-Lalanine amidase and having the following sequence:

MNRQPNNDPSTHFKMYKSGKKWVFAGLTAVTLLTASGAVAHADD (SEQ ID NO:5), corresponding to NCBI accession number AAP97061.1.

In some aspects, the secretory signal sequence comprises a secretion peptide derived from a L. reuteri F275 cell wall hydrolase peptidoglycan-binding domain-containing protein (LysM) having the following sequence:

MKLSKKVAKITAAITGAVALGTVATATTANAD (SEQ ID NO:6), corresponding to NCBI accession number WP_086135997.1.

In some aspects, the secretory signal sequence comprises a secretion peptide derived from a L. reuteri 6475 amino acid ABC transporter substrate-binding protein having the following sequence:

MGKFWKKALLTIAALTVGTSAGITSVSAASSAVNX (SEQ ID NO:7), corresponding to NCBI accession number WP_003667327.1.

In some aspects, the signal peptide amino acid sequence has, has at least, or has at most 5, 6, 7, 8, 9, 10, 11, 12, 13, 14, 15, 16, 17, 18, 19, 20, 21, 22, 23, 24, 25, 26, 27, 28, 29, or 30 amino acids, or any range or value derivable therein, and has, has at least, or has at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NOs:1-7. In some aspects, the signal peptide amino acid sequence comprises any of SEQ ID NOs:1-7. In some aspects, the signal peptide amino acid sequence consists of any of SEQ ID NOs:1-7.

In some aspects, expression of one or more biologically active peptides by a microorganism is achieved by operably linking a nucleic acid encoding the one or more biologically active peptides or portions thereof to a promoter and incorporating the construct into an expression vector, which is taken up and expressed by the microorganism. The expression vector is capable of expressing the heterologous biologically active peptides under conditions present at the intended site, e.g. such as in the gastrointestinal tract.

Usually, the expression system will comprise a genetic construct comprising at least one nucleotide sequence encoding the desired biologically active peptides, preferably operably linked to a promoter capable of directing expression of the sequence in the hosting microorganism. Suitably the peptide to be expressed can be encoded by a nucleic acid sequence that is adapted to the preferred codon usage of the host. The construct may further contain (all) other suitable element(s), including enhancers, transcription initiation sequences, signal sequences, reporter genes, transcription termination sequences, etc., operable in the selected host, as is known to the person skilled in the art. The construct is preferably in a form suitable for transformation of the host and/or in a form that can be stably maintained in the host, such as a vector, plasmid or mini-chromosome. Suitable vectors comprising nucleic acid for introduction into microorganisms, e.g. bacteria, can be chosen or constructed, containing appropriate regulatory sequences, including promoter sequences, terminator fragments, enhancer sequences, marker genes and other sequences as appropriate. In some aspects, a food-grade construct is used.

In one aspect, the genetic construct comprising at least one nucleotide sequence encoding the desired biologically active peptides is stably transfected into the microorganism, i.e. microorganisms in which the gene coding for the biologically active peptides has been integrated into the host's genome. Techniques for establishing stably transfected microorganisms are known in the art. For instance, the gene of interest may be cloned into the host's genome via homologous recombination. In some aspects, an essential gene of the host is disrupted by the homologous recombination event, such as deletion of the gene, one or more amino acid substitutions leading to an inactive form of the protein encoded by the essential gene, or to a frameshift mutation resulting in a truncated form of the protein encoded by the essential gene. The transforming plasmid can be any plasmid, as long as it cannot complement the disrupted essential gene.

The plasmid may be self-replicating, preferably carrying one or more genes of interest and one or more resistance markers, or the plasmid is an integrative plasmid. In the latter case, the integrative plasmid itself may be used to disrupt the essential gene, by causing integration at the locus of the essential gene because of which the function of the essential gene is disrupted. In some aspects, the essential gene is replaced by double homologous recombination by a cassette comprising the sequence of interest, flanked by targeting sequences that target the insertion to the essential gene. It will be appreciated that that these targeting sequences are sufficiently long and sufficiently homologous to enable integration of the gene of interest into the target site.

The genetic construct encoding the biologically active peptides disclosed herein may thus be present in the microorganism extra-chromosomally, for example, autonomously replicating using an own origin of replication, or may be integrated into the microbial genomic DNA, e.g., bacterial or yeast chromosome. In the latter case, a single or multiple copies of the said nucleic acid may be integrated; the integration may occur at a random site of the chromosome or, as described above, at a predetermined site thereof.

Hence, in one aspect, the genetic construct encoding the biologically active peptides disclosed herein may further comprise sequences configured to effect insertion of the said genetic construct into the genome, e.g., a chromosome, of a microorganism. In an example, insertion of the genetic construct into particular sites within a genome, e.g. chromosome, of a microorganism may be facilitated by homologous recombination. For instance, the genetic construct of the invention may comprise one or more regions of homology to the said site of integration within the genome e.g., a chromosome, of the microorganism. The sequence at the said genome, e.g. chromosome, site may be natural, i.e., as occurring in nature, or may be an exogenous sequence introduced by previous genetic engineering.

In some aspects, the genetic construct or vector used for the expression of ShK-235 by a recombinant microorganism comprises the following nucleotide sequence:

(SEQ ID NO: 8)
ATGGAGAAGAAAATTATTTCAGCTATTTTAATGTCAACTGTTATTTTATCA
GCTGCAGCTCCATTATCAGGTGTTTATGCTGCTGCACGAAGTTGTATTGATACGATTC
CAAAATCACGGTGTACCGCTTTTAAATGCAAGCACAGTATTAAGTACCGTTTGTCAT
TCTGCCGGAAAACTTGTGGAACTTGTGCTTAAGAATTCGGTACCCCGGGTTCGAAGG
CGCCAAGCTTCAAATTACAGCACGTGTTGCTTTGATTGATAGCCAAAAAGCAGCAGT
TGATAAAGCAATTACTGATATTGCTGAAAAATTGTAATTTATAAATAAAAATCACCT
TTTAGAGGTGGTTTTTTTATTTATAAATTATTCGTTTGATTTCGCTTTCGATAGAACA
ATCAAAGCGAGAATAAGGAAGATAAATCCCATAAGGGCGGGAGCAGAATGTCCGA
GACTAATTCATGAGATCGATTTTTTATTAAAACGTCTCAAAATCGTTTCTGAGACGTT
TTAGCGTTTATTTCGTTTAGTTATCGGCATAATCGTTAAAACAGGCGTTATCGTAGCG
TAAAAGCCCTTGAGCGTAGCGTGGCTTTGCAGCGAAGATGTTGTCTGTTAGATTATG
AAAGCCGATGACTGAATGAAATAATAAGCGCAGCGTCCTTCTATTTCGGTTGGAGG
AGGCTCAAGGGAGTTTGAGGGAATGAAATTCCCTCATGGGTTTGATTTTAAAAATTG
CTTGCAATTTTGCCGAGCGGTAGCGCTGGAAAATTTTTGAAAAAAATTTGGAATTTG
GAAAAAAATGGGGGGAAAGGAAGCGAATTTTGCTTCCGTACTACGACCCCCCATTA
AGTGCCGAGTGCCAATTTTTGTGCCAAAAACGCTCTATCCCAACTGGCTCAAGGGTT
TGAGGGGTTTTTCAATCGCCAACGAATCGCCAACGTTTTCGCCAACGTTTTTTATAA
ATCTATATTTAAGTAGCTTTATTGTTGTTTTTATGATTACAAAGTGATACACTAATTT
TATAAAATTATTTGATTGGAGTTTTTTAAATGGTGATTTCAGAATCGAAAAAAAGAG
TTATGATTTCTCTGACAAAAGAGCAAGATAAAAAATTAACAGATATGGCGAAACAA
AAAGGTTTTTCAAAATCTGCGGTTGCGGCGTTAGCTATAGAAGAATATGCAAGAAA
GGAATCAGAACAAAAAAAATAAGCGAAAGCTCGCGTTTTTAGAAGGATACGAGTTT
TCGCTACTTGTTTTTGATAAGGTAATTATATCATGGCTATTAAAAATACTAAAGCTA
GAAATTTTGGATTTTTATTATATCCTGACTCAATTCCTAATGATTGGAAAGAAAAATT
AGAGAGTTTGGGCGTATCTATGGCTGTCAGTCCTTTACACGATATGGACGAAAAAAA
AGATAAAGATACATGGAATAGTAGTGATGTTATACGAAATGGAAAGCACTATAAAA
AACCACACTATCACGTTATATATATTGCACGAAATCCTGTAACAATAGAAAGCGTTA
GGAACAAGATTAAGCGAAAATTGGGGAATAGTTCAGTTGCTCATGTTGAGATACTT
GATTATATCAAAGGTTCATATGAATATTTGACTCATGAATCAAAGGACGCTATTGCT
AAGAATAAACATATATACGACAAAAAAGATATTTTGAACATTAATGATTTTGATATT
GACCGCTATATAACACTTGATGAAAGCCAAAAAAGAGAATTGAAGAATTTACTTTT
AGATATAGTGGATGACTATAATTTGGTAAATACAAAAGATTTAATGGCTTTTATTCG
CCTTAGGGGAGCGGAGTTTGGAATTTTAAATACGAATGATGTAAAAGATATTGTTTC
AACAAACTCTAGCGCCTTTAGATTATGGTTTGAGGGCAATTATCAGTGTGGATATAG
AGCAAGTTATGCAAAGGTTCTTGATGCTGAAACGGGGGAAATAAAATGACAAACAA
AGAAAAAGAGTTATTTGCTGAAAATGAGGAATTAAAAAAAGAAATTAAGGACTTAA
AAGAGCGTATTGAAAGATACAGAGAAATGGAAGTTGAATTAAGTACAACAATAGAT
TTATTGAGAGGAGGGATTATTGAATAAATAAAAGCCCCCCTGACGAAAGTCGAAGG
GGGCTTTTATTTTGGTTTGATGTTGCGATTAATAGCAATACGATTGCAATAAACAAA
ACGATCCCCTTAGAAGCAAACTTAGAGTGTGTTGATAGTGCATTATCTTAAAATTTT
GTATAATAGGAATTGAAGTTAAATTAGATGCTAAAAATAGGAATTGAAGTTAAATT
AGATGCTAAAAATTTGTAATTAAGAAGGAGGGATTCGTCATGTTGGTATTCCAAATG
CGTAATGTAGATAAAACATCTACTGTTTTGAAACAGACTAAAAACAGTGATTACGCA
GATAAATAAATACGTTAGATTAATTCCTACCAGTGACTAATCTTATGACTTTTTAAA
CAGATAACTAAAATTACAAACAAATCGTTTAACTTCAGGAGAGATTACATGAACAA
AAATATAAAATATTCTCAAAACTTTTTAACGAGTGAAAAAGTACTCAACCAAATAAT
AAAACAATTGAATTTAAAAGAAACCGATACCGTTTACGAAATTGGAACAGGTAAAG
GGCATTTAACGACGAAACTGGCTAAAATAAGTAAACAGGTAACGTCTATTGAATTA
GACAGTCATCTATTCAACTTATCGTCAGAAAAATTAAAACTGAATACTCGTGTCACT
TTAATTCACCAAGATATTCTACAGTTTCAATTCCCTAACAAACAGAGGTATAAAATT
GTTGGGAATATTCCTTACAATTTAAGCACACAAATTATTAAAAAAGTGGTTTTTGAA
AGCCGTGCGTCTGACATCTATCTGACTGTTGAAGAAGGATTCTACAAGCGTACCTTG
GATATTCACCGAACACTAGGGTTGCTCTTGCACACTCAAGTCTCGATTCAGCAATTG
CTTAAGCTGCCAGCGGAATGCTTTCATCCTAAACCAAAAGTAAACAGTGTCTTAATA
AAACTTACCCGCCATACCACAGATGTTCCAGATAAATATTGGAAGCTATATAAGTAC
TTTGTTTCAAAATGGGTCAATCGAGAATATCGTCAACTGTTTACTAAAAATCAGTTT
CGTCAAGCAATGAAACACGCCAAAGTAAACAATTTAAGTACCATTACTTATGAGCA
AGTATTGTCTATTTTTAATAGTTATCTATTATTTAACGGGAGGAAATAATTCTATGAG
TCGCTTTTTTAAATTTGGAAAGTTACACGTTACTAAAGGGAATGGAGACCGGGGTCG
ACCCTTCAATAGAGTTCTTAACGTTAATCCGAAAAAAACTAACGTTAATATTAAAAA
ATAAGATCCGCTTGTGAATTATGTATAATTTGATTAGACTAAAGAATAGGAGAAAGT
ATGATGATATTTAAAAAACTTTCTCGTTAAGATAGGTTGTTGGTGAGCATGTTATAT
ACGGATGTATCGGTTTCCTTAATGCAAAATTTTGTTGCTATCTTATTAATTTTTCTATT
ATATAGATATATTCAAAGAAAGATAACATTTAAACGGATCATATTAGATATTTTAAT
AGCGATTATTTTTTCAATATTATATCTGTTTATTTCAGATGCGTCATTACTTGTAATG
GTATTAATGCGATTAGGGTGGCATTTTCATCAACAAAAAGAAAATAAGATAAAAAC
GACTGATACAGCTAATTTAATTCTAATTATCGTGATCCAGTTATTGTTAGTTGCGGTT
GGGACTATTATTAGTCAGTTTACCATATCGATTATCAAAAGTGATTTCAGCCAAAAT
ATATTGAACAATAGTGCAACAGATATAACTTTATTAGGTATTTTCTTTGCTGTTTTAT
TTGACGGCTTGTTCTTTATATTATTGAAGAATAAGCGGACTGAATTACAACATTTAA
ATCAAGAAATCATTGAATTTTCGTTAGAAAAACAATATTTTATATTTATATTTATTTT
ATTTATAGTAATAGAAATTATTTTAGCAGTTGGGAATCTTCAAGGAGTAACAGCCAC
GATATTATTAACCATTATCATTATTTTTTGTGTCCTTATCGGGATGACTTTTTGGCAA
GTGATGCTTTTTTTGAAGGCTTATTCGATTCGCCAAGAAGCCAATGACCAATTGGTC
CGGAATCAACAACTTCAAGATTATCTAGTCAATATCGAACAGCAGTACACCGAATTA
CGGCGATTTAAGCATGATTATCAAAACATCTTATTATCGTTGGAGAGTTTTGCCGAA
AAGGGCGATCAGCAACAGTTTAAGGCGTATTACCAAGAATTATTAGCACAACGGCC
AATTCAAAGTGAAATCCAAGGGGCAGTCATTGCACAACTCGACTACTTGAAAAATG
ATCCTATTCGAGGATTAGTCATTCAAAAGTTTTTGGCAGCCAAACAGGCTGGTGTTA
CTTTAAAATTCGAAATGACCGAACCAATCGAATTAGCAACCGCTAATCTATTAACGG
TTATTCGGATTATCGGTATTTTATTAGACAATGCGATTGAACAAGCCGTTCAAGAAA
CCGATCAATTGGTGAGTTGTGCTTTCTTACAATCTGATGGTTTAATCGAAATTACGAT
TGAAAATACGGCCAGTCAAGTTAAGAATCTCCAAGCATTTTCAGAGTTAGGCTATTC
AACGAAAGGCGCTGGTCGGGGGACTGGTTTAGCTAATGTGCAGGATTTGATTGCCA
AACAAACCAATTTATTCTTAGAAACACAGATTGAAAATAGAAAGTTACGACAGACA
TTGATGATTACGGAGGAAACTTAATTTGTATCCCGTTTATTTATTAGAGGATGATTTA
CAGCAACAAGCGATTTATCAGCAAATTATCGCGAATACGATTATGATTAACGAATTT
GCAATGACTTTAACATGCGCTGCCAGTGATACTGAGACATTGTTGGCGGCAATTAAG
GATCAGCAACGAGGTTTATTCTTTTTGGATATGGAAATTGAGGATAACCGCCAAGCC
GGTTTAGAAGTGGCAACTAAGATTCGGCAGATGATGCCGTTTGCGCAAATTGTCTTC
ATTACAACCCACGAGGAACTGACATTATTAACGTTAGAACGAAAAATAGCGCCTTT
AGATTACATTCTCAAGGACCAAACAATGGCTGAAATCAAAAGGCAATTGATTGATG
ATCTATTGTTAGCTGAGAAGCAAAACGAGGCGGCAGCGTATCACCGAGAAAATTTA
TTTAGTTATAAAATAGGTCCTCGCTTTTTCTCATTACCATTAAAGGAAGTTGTTTATT
TATATACTGAAAAAGAAAATCCGGGTCATATTAATTTGTTAGCCGTTACCAGAAAGG
TTACTTTTCCAGGAAATTTAAATGCGCTGGAAGCCCAATATCCAATGCTCTTTCGGT
GTGATAAAAGTTACTTAGTTAACCTATCTAATATTGCCAATTATGACAGTAAAACAC
GGAGTTTAAAATTTGTAGATGGCAGTGAGGCAAAAGTCTCGTTCCGGAAATCACGG
GAACTAGTGGCCAAATTAAAACAAATGATGTAGCGCCTGCAGGCACGCCAAATGAT
CCCAGTAAAAAGCCACCCGCATGGCGGGTGGCTTTTTATTAGCCCTAGAAGGGCTTC
CCACACGCATTTCAGCGCCTTAGTGCCTTAGTTTGTGAATCATAGGTGGTATAGTCC
CGAAATACCCGTCTAAGGAATTGTCAGATAGGCCTAATGACTGGCTTTTATAATATG
AGATAATGCCGACTGTACTTTTTACAGTCGGTTTTCTAATGTCACTAACCTGCCCCGT
TAGTTGAAGAAGGTTTTTATATTACAGCTCCAGATCTACCGGTTTAATTTGAAAATT
GATATTAGCGTTTAACAGTTAAATTAATACGTTAATAATTTTTTTGTCTTTAAATAGG
GATTTGAAGCATAATGGTGTTATAGCGTACTTAGCTGGCCAGCATATATGTATTCTA
TAAAATACTATTACAAGGAGATTTTAGCC.

In some aspects, the genetic construct or vector used for the expression of ShK-235 by a recombinant microorganism has, has at least, or has at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:8. In some aspects, the genetic construct or vector used for the expression of ShK-235 by a recombinant microorganism comprises SEQ ID NO:8. In some aspects, the genetic construct or vector used for the expression of ShK-235 by a recombinant microorganism consists of SEQ ID NO:8.

In some aspects, the genetic construct or vector used for the expression of IbTX by a recombinant microorganism comprises the following nucleotide sequence:

(SEQ ID NO: 9)
AAAGTAAACAGTGTCTTAATAAAACTTACCCGCCATACCACAGATGTTCC
AGATAAATATTGGAAGCTATATAAGTACTTTGTTTCAAAATGGGTCAATCGAGAATA
TCGTCAACTGTTTACTAAAAATCAGTTTCGTCAAGCAATGAAACACGCCAAAGTAAA
CAATTTAAGTACCATTACTTATGAGCAAGTATTGTCTATTTTTAATAGTTATCTATTA
TTTAACGGGAGGAAATAATTCTATGAGTCGCTTTTTTAAATTTGGAAAGTTACACGT
TACTAAAGGGAATGGAGACCGGGGTCGACCCTTCAATAGAGTTCTTAACGTTAATCC
GAAAAAAACTAACGTTAATATTAAAAAATAAGATCCGCTTGTGAATTATGTATAATT
TGATTAGACTAAAGAATAGGAGAAAGTATGATGATATTTAAAAAACTTTCTCGTTAA
GATAGGTTGTTGGTGAGCATGTTATATACGGATGTATCGGTTTCCTTAATGCAAAAT
TTTGTTGCTATCTTATTAATTTTTCTATTATATAGATATATTCAAAGAAAGATAACAT
TTAAACGGATCATATTAGATATTTTAATAGCGATTATTTTTTCAATATTATATCTGTT
TATTTCAGATGCGTCATTACTTGTAATGGTATTAATGCGATTAGGGTGGCATTTTCAT
CAACAAAAAGAAAATAAGATAAAAACGACTGATACAGCTAATTTAATTCTAATTAT
CGTGATCCAGTTATTGTTAGTTGCGGTTGGGACTATTATTAGTCAGTTTACCATATCG
ATTATCAAAAGTGATTTCAGCCAAAATATATTGAACAATAGTGCAACAGATATAACT
TTATTAGGTATTTTCTTTGCTGTTTTATTTGACGGCTTGTTCTTTATATTATTGAAGAA
TAAGCGGACTGAATTACAACATTTAAATCAAGAAATCATTGAATTTTCGTTAGAAAA
ACAATATTTTATATTTATATTTATTTTATTTATAGTAATAGAAATTATTTTAGCAGTT
GGGAATCTTCAAGGAGTAACAGCCACGATATTATTAACCATTATCATTATTTTTTGT
GTCCTTATCGGGATGACTTTTTGGCAAGTGATGCTTTTTTTGAAGGCTTATTCGATTC
GCCAAGAAGCCAATGACCAATTGGTCCGGAATCAACAACTTCAAGATTATCTAGTC
AATATCGAACAGCAGTACACCGAATTACGGCGATTTAAGCATGATTATCAAAACAT
CTTATTATCGTTGGAGAGTTTTGCCGAAAAGGGCGATCAGCAACAGTTTAAGGCGTA
TTACCAAGAATTATTAGCACAACGGCCAATTCAAAGTGAAATCCAAGGGGCAGTCA
TTGCACAACTCGACTACTTGAAAAATGATCCTATTCGAGGATTAGTCATTCAAAAGT
TTTTGGCAGCCAAACAGGCTGGTGTTACTTTAAAATTCGAAATGACCGAACCAATCG
AATTAGCAACCGCTAATCTATTAACGGTTATTCGGATTATCGGTATTTTATTAGACA
ATGCGATTGAACAAGCCGTTCAAGAAACCGATCAATTGGTGAGTTGTGCTTTCTTAC
AATCTGATGGTTTAATCGAAATTACGATTGAAAATACGGCCAGTCAAGTTAAGAATC
TCCAAGCATTTTCAGAGTTAGGCTATTCAACGAAAGGCGCTGGTCGGGGGACTGGTT
TAGCTAATGTGCAGGATTTGATTGCCAAACAAACCAATTTATTCTTAGAAACACAGA
TTGAAAATAGAAAGTTACGACAGACATTGATGATTACGGAGGAAACTTAATTTGTAT
CCCGTTTATTTATTAGAGGATGATTTACAGCAACAAGCGATTTATCAGCAAATTATC
GCGAATACGATTATGATTAACGAATTTGCAATGACTTTAACATGCGCTGCCAGTGAT
ACTGAGACATTGTTGGCGGCAATTAAGGATCAGCAACGAGGTTTATTCTTTTTGGAT
ATGGAAATTGAGGATAACCGCCAAGCCGGTTTAGAAGTGGCAACTAAGATTCGGCA
GATGATGCCGTTTGCGCAAATTGTCTTCATTACAACCCACGAGGAACTGACATTATT
AACGTTAGAACGAAAAATAGCGCCTTTAGATTACATTCTCAAGGACCAAACAATGG
CTGAAATCAAAAGGCAATTGATTGATGATCTATTGTTAGCTGAGAAGCAAAACGAG
GCGGCAGCGTATCACCGAGAAAATTTATTTAGTTATAAAATAGGTCCTCGCTTTTTC
TCATTACCATTAAAGGAAGTTGTTTATTTATATACTGAAAAAGAAAATCCGGGTCAT
ATTAATTTGTTAGCCGTTACCAGAAAGGTTACTTTTCCAGGAAATTTAAATGCGCTG
GAAGCCCAATATCCAATGCTCTTTCGGTGTGATAAAAGTTACTTAGTTAACCTATCT
AATATTGCCAATTATGACAGTAAAACACGGAGTTTAAAATTTGTAGATGGCAGTGA
GGCAAAAGTCTCGTTCCGGAAATCACGGGAACTAGTGGCCAAATTAAAACAAATGA
TGTAGCGCCTGCAGGCACGCCAAATGATCCCAGTAAAAAGCCACCCGCATGGCGGG
TGGCTTTTTATTAGCCCTAGAAGGGCTTCCCACACGCATTTCAGCGCCTTAGTGCCTT
AGTTTGTGAATCATAGGTGGTATAGTCCCGAAATACCCGTCTAAGGAATTGTCAGAT
AGGCCTAATGACTGGCTTTTATAATATGAGATAATGCCGACTGTACTTTTTACAGTC
GGTTTTCTAATGTCACTAACCTGCCCCGTTAGTTGAAGAAGGTTTTTATATTACAGCT
CCAGATCTACCGGTTTAATTTGAAAATTGATATTAGCGTTTAACAGTTAAATTAATA
CGTTAATAATTTTTTTGTCTTTAAATAGGGATTTGAAGCATAATGGTGTTATAGCGTA
CTTAGCTGGCCAGCATATATGTATTCTATAAAATACTATTACAAGGAGATTTTAGCC
ATGGAGAAGAAAATTATTTCAGCTATTTTAATGTCAACTGTTATTTTATCAGCTGCA
GCTCCATTATCAGGTGTTTATGCTGCTGCAGAATTTACTGATGTTGATTGTAGTGTTA
GTAAAGAATGTTGGAGTGTTTGTAAAGATTTATTTGGTGTTGATCGTGGTAAATGTA
TGGGTAAAAAATGTCGTTGTTATCAATAAGAATTCGGTACCCCGGGTTCGAAGGCGC
CAAGCTTCAAATTACAGCACGTGTTGCTTTGATTGATAGCCAAAAAGCAGCAGTTGA
TAAAGCAATTACTGATATTGCTGAAAAATTGTAATTTATAAATAAAAATCACCTTTT
AGAGGTGGTTTTTTTATTTATAAATTATTCGTTTGATTTCGCTTTCGATAGAACAATC
AAAGCGAGAATAAGGAAGATAAATCCCATAAGGGCGGGAGCAGAATGTCCGAGAC
TAATTCATGAGATCGATTTTTTATTAAAACGTCTCAAAATCGTTTCTGAGACGTTTTA
GCGTTTATTTCGTTTAGTTATCGGCATAATCGTTAAAACAGGCGTTATCGTAGCGTA
AAAGCCCTTGAGCGTAGCGTGGCTTTGCAGCGAAGATGTTGTCTGTTAGATTATGAA
AGCCGATGACTGAATGAAATAATAAGCGCAGCGTCCTTCTATTTCGGTTGGAGGAG
GCTCAAGGGAGTTTGAGGGAATGAAATTCCCTCATGGGTTTGATTTTAAAAATTGCT
TGCAATTTTGCCGAGCGGTAGCGCTGGAAAATTTTTGAAAAAAATTTGGAATTTGGA
AAAAAATGGGGGGAAAGGAAGCGAATTTTGCTTCCGTACTACGACCCCCCATTAAG
TGCCGAGTGCCAATTTTTGTGCCAAAAACGCTCTATCCCAACTGGCTCAAGGGTTTG
AGGGGTTTTTCAATCGCCAACGAATCGCCAACGTTTTCGCCAACGTTTTTTATAAATC
TATATTTAAGTAGCTTTATTGTTGTTTTTATGATTACAAAGTGATACACTAATTTTAT
AAAATTATTTGATTGGAGTTTTTTAAATGGTGATTTCAGAATCGAAAAAAAGAGTTA
TGATTTCTCTGACAAAAGAGCAAGATAAAAAATTAACAGATATGGCGAAACAAAAA
GGTTTTTCAAAATCTGCGGTTGCGGCGTTAGCTATAGAAGAATATGCAAGAAAGGA
ATCAGAACAAAAAAAATAAGCGAAAGCTCGCGTTTTTAGAAGGATACGAGTTTTCG
CTACTTGTTTTTGATAAGGTAATTATATCATGGCTATTAAAAATACTAAAGCTAGAA
ATTTTGGATTTTTATTATATCCTGACTCAATTCCTAATGATTGGAAAGAAAAATTAGA
GAGTTTGGGCGTATCTATGGCTGTCAGTCCTTTACACGATATGGACGAAAAAAAAGA
TAAAGATACATGGAATAGTAGTGATGTTATACGAAATGGAAAGCACTATAAAAAAC
CACACTATCACGTTATATATATTGCACGAAATCCTGTAACAATAGAAAGCGTTAGGA
ACAAGATTAAGCGAAAATTGGGGAATAGTTCAGTTGCTCATGTTGAGATACTTGATT
ATATCAAAGGTTCATATGAATATTTGACTCATGAATCAAAGGACGCTATTGCTAAGA
ATAAACATATATACGACAAAAAAGATATTTTGAACATTAATGATTTTGATATTGACC
GCTATATAACACTTGATGAAAGCCAAAAAAGAGAATTGAAGAATTTACTTTTAGAT
ATAGTGGATGACTATAATTTGGTAAATACAAAAGATTTAATGGCTTTTATTCGCCTT
AGGGGAGCGGAGTTTGGAATTTTAAATACGAATGATGTAAAAGATATTGTTTCAAC
AAACTCTAGCGCCTTTAGATTATGGTTTGAGGGCAATTATCAGTGTGGATATAGAGC
AAGTTATGCAAAGGTTCTTGATGCTGAAACGGGGGAAATAAAATGACAAACAAAGA
AAAAGAGTTATTTGCTGAAAATGAGGAATTAAAAAAAGAAATTAAGGACTTAAAAG
AGCGTATTGAAAGATACAGAGAAATGGAAGTTGAATTAAGTACAACAATAGATTTA
TTGAGAGGAGGGATTATTGAATAAATAAAAGCCCCCCTGACGAAAGTCGAAGGGGG
CTTTTATTTTGGTTTGATGTTGCGATTAATAGCAATACGATTGCAATAAACAAAACG
ATCCCCTTAGAAGCAAACTTAGAGTGTGTTGATAGTGCATTATCTTAAAATTTTGTAT
AATAGGAATTGAAGTTAAATTAGATGCTAAAAATAGGAATTGAAGTTAAATTAGAT
GCTAAAAATTTGTAATTAAGAAGGAGGGATTCGTCATGTTGGTATTCCAAATGCGTA
ATGTAGATAAAACATCTACTGTTTTGAAACAGACTAAAAACAGTGATTACGCAGAT
AAATAAATACGTTAGATTAATTCCTACCAGTGACTAATCTTATGACTTTTTAAACAG
ATAACTAAAATTACAAACAAATCGTTTAACTTCAGGAGAGATTACATGAACAAAAA
TATAAAATATTCTCAAAACTTTTTAACGAGTGAAAAAGTACTCAACCAAATAATAAA
ACAATTGAATTTAAAAGAAACCGATACCGTTTACGAAATTGGAACAGGTAAAGGGC
ATTTAACGACGAAACTGGCTAAAATAAGTAAACAGGTAACGTCTATTGAATTAGAC
AGTCATCTATTCAACTTATCGTCAGAAAAATTAAAACTGAATACTCGTGTCACTTTA
ATTCACCAAGATATTCTACAGTTTCAATTCCCTAACAAACAGAGGTATAAAATTGTT
GGGAATATTCCTTACAATTTAAGCACACAAATTATTAAAAAAGTGGTTTTTGAAAGC
CGTGCGTCTGACATCTATCTGACTGTTGAAGAAGGATTCTACAAGCGTACCTTGGAT
ATTCACCGAACACTAGGGTTGCTCTTGCACACTCAAGTCTCGATTCAGCAATTGCTT
AAGCTGCCAGCGGAATGCTTTCATCCTAAACCA.

In some aspects, the genetic construct or vector used for the expression of IbTX by a recombinant microorganism has, has at least, or has at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:9. In some aspects, the genetic construct or vector used for the expression of IbTX by a recombinant microorganism comprises SEQ ID NO:9. In some aspects, the genetic construct or vector used for the expression of IbTX by a recombinant microorganism consists of SEQ ID NO:9.

### 1. Vectors

Expression vectors can be suitable for replication and, in some aspects, integration. Typical cloning vectors contain transcription and translation terminators, initiation sequences, and promoters useful for regulation of the expression of the desired nucleic acid sequence. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

In certain aspects the expression vector may be provided to a cell in the form of a viral vector. Viral vector technology is well known in the art and is described, for example, in Sambrook et al. (2001) Molecular Cloning: A Laboratory Manual, Cold Spring Harbor Laboratory, New York), and in other virology and molecular biology manuals. A number of viral based systems have been developed for gene transfer into mammalian cells. Viruses that are useful as vectors include, but are not limited to, retroviruses, adenoviruses, adeno-associated viruses, herpes viruses, and lentiviruses (including self-inactivating lentivirus vectors). For example, adenoviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. Thus, in some aspects, the nucleic acid encoding the one or more biologically active peptides is introduced into a microorganism using a recombinant vector such as a viral vector including, for example, a lentivirus, a retrovirus, gamma-retroviruses, an adeno-associated virus (AAV), a herpesvirus, or an adenovirus.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a microorganism, the vector can either be stably replicated by the cells during mitosis as an autonomous structure, incorporated within the genome of the microorganism, or maintained in the microorganism's nucleus or cytoplasm.

Expression cassettes included in the vectors particularly contain (in a 5′-to-3′ direction) regulatory elements including a transcriptional promoter operably linked to a protein-coding sequence, splice signals including intervening sequences, a transcriptional termination/polyadenylation sequence, post-transcriptional regulatory elements, and origins of replication.

a. Promoter/Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

In some aspects, a promoter employed is expressed constitutively in the microorganism. The use of a constitutive promoter avoids the need to supply an inducer or other regulatory signal for expression to take place. In some aspects, the promoter directs expression at a level at which the microorganism remains viable, i.e. retains some metabolic activity, even if growth is not maintained.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the thymidine kinase promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each specifically incorporated by reference herein in its entirety). Furthermore, it is contemplated the control sequences that direct transcription and/or expression of sequences within non-nuclear organelles such as mitochondria and the like can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, specifically incorporated by reference herein in its entirety). The promoters employed may be constitutive, cell-specific, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, through world wide web at epd.isb-sib.ch/) could also be used to drive expression. Non-limiting examples of other potential promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). A specific example could be a phosphoglycerate kinase (PGK) promoter.

In some aspects, expression of the polynucleotide is regulated by a constitutive promoter. In some aspects, the constitutive promoter is EF-Tu, T7, Sp6, or lac. In some aspects, the constitutive promoter is CAG (also known as CAGGS or CBA), EF-1ALPHA, ubiquitin, or CMV.

In some aspects, expression of the polynucleotide is regulated by a promoter that is regulated by oxygen levels. In some aspects, expression of the polynucleotide is regulated by a promoter that is turned on under hypoxic, micro-oxic, or anaerobic conditions. In some aspects, expression of the polynucleotide is regulated promoters regulated by FNR, as an example.

b. Protease Cleavage Sites/Self-Cleaving Peptides and Internal Ribosome Binding Sites

Suitable protease cleavages sites and self-cleaving peptides are known to the skilled person (see, e.g., in Ryan et al., 1997; Scymczak et al., 2004). Examples of protease cleavage sites are the cleavage sites of furin proteases, potyvirus NIa proteases (e.g., tobacco etch virus protease), potyvirus HC proteases, potyvirus P1 (P35) proteases, byovirus Nla proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PY\IF (parsnip yellow fleck virus) 3C-like protease, thrombin, factor Xa and enterokinase. Due to its high cleavage stringency, TEV (tobacco etch virus) protease cleavage sites may be used. In some aspects, the protease cleavage sites are the cleavage sites of furin proteases.

Exemplary self-cleaving peptides (also called “cis-acting hydrolytic elements”, CHYSEL; see deFelipe (2002) are derived from potyvirus and cardiovirus 2A peptides. Particular self-cleaving peptides may be selected from 2A peptides derived from FMDV (foot-and-mouth disease virus), equine rhinitis A virus, Thoseà asigna virus, and porcine teschovirus.

A specific initiation signal also may be used for efficient translation of coding sequences in a polycistronic message. These signals include the ATG initiation codon or adjacent sequences. For example, an initiation signal may comprise a Kozak consensus sequence having an amino acid sequence comprising GCCACCAUGGG. See Kozak, 1987; Harte et al., 2012. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain aspects, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, specifically incorporated by reference herein in their entirety). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs may comprise at least one termination signal. A “termination signal” or “terminator” is comprised of the DNA sequences involved in specific termination of an RNA transcript by an RNA polymerase. Thus, in certain aspects a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other aspects involving eukaryotes, the terminator comprises a signal for the cleavage of the RNA, and the terminator signal promotes polyadenylation of the message. The terminator and/or polyadenylation site elements can serve to enhance message levels and to minimize read through from the cassette into other sequences.

Terminators contemplated include any known terminator of transcription described herein or known to one of ordinary skill in the art, including but not limited to, for example, the termination sequences of genes, such as for example the bovine growth hormone terminator or viral termination sequences, such as for example the SV40 terminator. In certain aspects, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice, and any such sequence may be employed. Exemplary aspects include the SV40 polyadenylation signal or the bovine growth hormone polyadenylation signal, convenient and known to function well in various target cells. Polyadenylation may increase the stability of the transcript or may facilitate cytoplasmic transport.

g. Post-Transcriptional Regulatory Elements

A vector for use in the disclosure can also comprise one or more post-transcriptional regulatory elements (PREs). Examples of PREs include the woodchuck hepatitis virus PRE (WPRE), hepatitis B virus PRE, and Intron A of human cytomegalovirus immediate early gene. See Sun et al. 2009 and Mariati et al. 2010 for further examples and details. In a particular aspect, the PRE is a WPRE. WPRE is a DNA sequence that, when transcribed, creates a tertiary structure to enhance expression of genes delivered by viral vectors.

h. Origins of Replication

In order to propagate a vector in a microorganism, the vector may contain one or more origins of replication sites (often termed “ori”), for example, a nucleic acid sequence corresponding to oriP of EBV as described above or a genetically engineered oriP with a similar or elevated function in differentiation programming, which is a specific nucleic acid sequence at which replication is initiated. Alternatively a replication origin of other extra-chromosomally replicating virus as described above or an autonomously replicating sequence (ARS) can be employed.

### 2. Vector Delivery

Genetic modification or introduction of exogenous nucleic acids into the microorganisms may use any suitable methods for nucleic acid delivery for transformation of a cell, as described herein or as would be known to one of ordinary skill in the art. Methods of introducing and expressing polynucleotides into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Such methods include, but are not limited to, direct delivery of DNA such as by ex vivo transfection (Wilson et al., 1989, Nabel et al, 1989); transduction; viral transduction; injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by nucleofection; by lipofection or liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile or nanoparticle bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985); thermal shock (Froger and Hall, 2007); and any combination of such methods. Through the application of techniques such as these, organelle(s), cell(s), tissue(s) or organism(s) may be stably or transiently transformed.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al. (2001) Molecular Cloning: A Laboratory Manual, Cold Spring Harbor Laboratory, New York).

Biological methods for introducing a polynucleotide of interest into a host cell can include the use of DNA and RNA vectors into which the polynucleotide of interest, or transgene, can be inserted. Viral vectors have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362, and the like).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. Also contemplated are nanoparticles. An illustrative colloidal system for use as a delivery vehicle in vitro and in vivo is a liposome (e.g., an artificial membrane vesicle).

Gene therapy methods and methods of delivering genes to subjects, for example using adeno-associated viruses, are described in U.S. Pat. No. 6,967,018, WO2014/093622, US2008/0175845, US 2014/0100265, EP2432490, EP2352823, EP2384200, WO2014/127198, WO2005/122723, WO2008/137490, WO2013/142114, WO2006/128190, WO2009/134681, EP2341068, WO2008/027084, WO2009/054994, WO2014059031, U.S. Pat. No. 7,977,049 and WO 2014/059029, each of which are specifically incorporated herein by reference in their entirety.

a. Liposome-Mediated Transfection

One illustrative delivery vehicle is a lipid and/or a liposome. The use of lipid formulations is contemplated for the introduction of the nucleic acids into a host cell (in vitro, ex vivo, or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In a certain aspect, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The amount of liposomes used may vary upon the nature of the liposome as well as the cell used, for example, about 5 to about 20 pg vector DNA per 1 to 10 million of cells may be contemplated.

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain aspects, a liposome may be complexed with a hemagglutinating virus (HVJ). This has been shown to facilitate fusion with the cell membrane and promote cell entry of liposome-encapsulated DNA (Kaneda et al., 1989). In other aspects, a liposome may be complexed or employed in conjunction with nuclear non-histone chromosomal proteins (HMG-1) (Kato et al., 1991). In yet further aspects, a liposome may be complexed or employed in conjunction with both HVJ and HMG-1. In other aspects, a delivery vehicle may comprise a ligand and a liposome.

In various aspects lipids suitable for use can be obtained from commercial sources. For example, lipofectamine can be obtained from Thermo Fisher Scientific, Waltham, Mass.; dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform can be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al. (1991) Glycobiology 5: 505-510). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

b. Electroporation

In certain aspects, a nucleic acid is introduced into a cell via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 μg vector DNA per 1 to 10 million of cells may be contemplated.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

c. Calcium Phosphate

In other aspects, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

d. DEAE-Dextran

In another aspect, a nucleic acid is delivered into a cell using DEAE-dextran followed by polyethylene glycol. In this manner, reporter plasmids were introduced into mouse myeloma and erythroleukemia cells (Gopal, 1985).

e. Selectable or Screenable Markers

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

In certain aspects, cells containing an exogenous nucleic acid may be identified in vitro or in vivo by including a marker in the expression vector or the exogenous nucleic acid. Such markers would confer an identifiable change to the cell permitting easy identification of cells containing the expression vector. Generally, a selection marker may be one that confers a property that allows for selection. A positive selection marker may be one in which the presence of the marker allows for its selection, while a negative selection marker is one in which its presence prevents its selection. An example of a positive selection marker is a drug resistance marker.

In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Selectable markers may include a type of reporter gene used in laboratory microbiology, molecular biology, and genetic engineering to indicate the success of a transfection or other procedure meant to introduce foreign DNA into a cell. Selectable markers are often antibiotic resistance genes; cells that have been subjected to a procedure to introduce foreign DNA are grown on a medium containing an antibiotic, and those cells that can grow have successfully taken up and expressed the introduced genetic material. Examples of selectable markers include: the Abicr gene or Neo gene from Tn5, which confers antibiotic resistance to geneticin.

A screenable marker may comprise a reporter gene, which allows the researcher to distinguish between wanted and unwanted cells. Certain aspects of the present invention utilize reporter genes to indicate specific cell lineages. For example, the reporter gene can be located within expression elements and under the control of the ventricular- or atrial-selective regulatory elements normally associated with the coding region of a ventricular- or atrial-selective gene for simultaneous expression. A reporter allows the cells of a specific lineage to be isolated without placing them under drug or other selective pressures or otherwise risking cell viability.

Examples of such reporters include genes encoding cell surface proteins (e.g., CD4, HA epitope), fluorescent proteins, antigenic determinants and enzymes (e.g., β-galactosidase). The vector containing cells may be isolated, e.g., by FACS using fluorescently-tagged antibodies to the cell surface protein or substrates that can be converted to fluorescent products by a vector encoded enzyme.

In specific aspects, the reporter gene is a fluorescent protein. A broad range of fluorescent protein genetic variants have been developed that feature fluorescence emission spectral profiles spanning almost the entire visible light spectrum (see below table for non-limiting examples). Mutagenesis efforts in the original Aequorea victoria jellyfish green fluorescent protein have resulted in new fluorescent probes that range in color from blue to yellow, and are some of the most widely used in vivo reporter molecules in biological research. Longer wavelength fluorescent proteins, emitting in the orange and red spectral regions, have been developed from the marine anemone, Discosoma striata, and reef corals belonging to the class Anthozoa. Still other species have been mined to produce similar proteins having cyan, green, yellow, orange, and deep red fluorescence emission. Developmental research efforts are ongoing to improve the brightness and stability of fluorescent proteins, thus improving their overall usefulness.

TABLE
Fluorescent Protein Properties

| | Excitation | Emission | Molar | | | Relative |
|---|---|---|---|---|---|---|
| Protein | Maximum | Maximum | Extinction | Quantum | in vivo | Brightness |
| (Acronym) | (nm) | (nm) | Coefficient | Yield | Structure | (% of EGFP) |
| [symbol] GFP (wt) | 395/475 | 509 | 21,000 | 0.77 | Monomer* | 48 |

Green Fluorescent Proteins

| | | | | | | |
|---|---|---|---|---|---|---|
| .EGFP | 484 | 507 | 56,000 | 0.60 | Monomer* | 100 |
| .AcGFP | 480 | 505 | 50,000 | 0.55 | Monomer* | 82 |
| [symbol] TurboGFP | 482 | 502 | 70,000 | 0.53 | Monomer* | 110 |
| [symbol] Emerald | 487 | 509 | 57,500 | 0.68 | Monomer* | 116 |
| [symbol] Azami Green | 492 | 505 | 55,000 | 0.74 | Monomer | 121 |
| [symbol] ZsGreen | 493 | 505 | 43,000 | 0.91 | Tetramer | 117 |

Blue Fluorescent Proteins

| | | | | | | |
|---|---|---|---|---|---|---|
| [symbol] EBFP | 383 | 445 | 29,000 | 0.31 | Monomer* | 27 |
| [symbol] Sapphire | 399 | 511 | 29,000 | 0.64 | Monomer* | 55 |
| .T-Sapphire | 399 | 511 | 44,000 | 0.60 | Monomer* | 79 |

Cyan Fluorescent Proteins

| | | | | | | |
|---|---|---|---|---|---|---|
| [symbol] ECFP | 439 | 476 | 32,500 | 0.40 | Monomer* | 39 |
| [symbol] mCFP | 433 | 475 | 32,500 | 0.40 | Monomer | 39 |
| [symbol] Cerulean | 433 | 475 | 43,000 | 0.62 | Monomer* | 79 |
| [symbol] CyPet | 435 | 477 | 35,000 | 0.51 | Monomer* | 53 |
| [symbol] AmCyan1 | 458 | 489 | 44,000 | 0.24 | Tetramer | 31 |
| .Midori-Ishi Cyan | 472 | 495 | 27,300 | 0.90 | Dimer | 73 |
| .mTFP1 (Teal) | 462 | 492 | 64,000 | 0.85 | Monomer | 162 |

Yellow Fluorescent Proteins

| | | | | | | |
|---|---|---|---|---|---|---|
| [symbol] EYFP | 514 | 527 | 83,400 | 0.61 | Monomer* | 151 |
| .Topaz | 514 | 527 | 94,500 | 0.60 | Monomer* | 169 |
| [symbol] Venus | 515 | 528 | 92,200 | 0.57 | Monomer* | 156 |
| .mCitrine | 516 | 529 | 77,000 | 0.76 | Monomer | 174 |
| [symbol] YPet | 517 | 530 | 104,000 | 0.77 | Monomer* | 238 |
| .PhiYFP | 525 | 537 | 124,000 | 0.39 | Monomer* | 144 |
| [symbol] ZsYellow1 | 529 | 539 | 20,200 | 0.42 | Tetramer | 25 |
| [symbol] mBanana | 540 | 553 | 6,000 | 0.7 | Monomer | 13 |

.Orange and Red Fluorescent Proteins

| | | | | | | |
|---|---|---|---|---|---|---|
| [symbol] Kusabira Orange | 548 | 559 | 51,600 | 0.60 | Monomer | 92 |
| .mOrange | 548 | 562 | 71,000 | 0.69 | Monomer | 146 |
| [symbol] dTomato | 554 | 581 | 69,000 | 0.69 | Dimer | 142 |
| .dTomato-Tandem | 554 | 581 | 138,000 | 0.69 | Monomer | 283 |
| .DsRed | 558 | 583 | 75,000 | 0.79 | Tetramer | 176 |
| .DsRed2 | 563 | 582 | 43,800 | 0.55 | Tetramer | 72 |
| [symbol] DsRed-Express (T1) | 555 | 584 | 38,000 | 0.51 | Tetramer | 58 |
| [symbol] DsRed-Monomer | 556 | 586 | 35,000 | 0.10 | Monomer | 10 |
| .mTangerine | 568 | 585 | 38,000 | 0.30 | Monomer | 34 |
| [symbol] mStrawberry | 574 | 596 | 90,000 | 0.29 | Monomer | 78 |
| [symbol] AsRed2 | 576 | 592 | 56,200 | 0.05 | Tetramer | 8 |
| .mRFP1 | 584 | 607 | 50,000 | 0.25 | Monomer | 37 |
| [symbol] JRed | 584 | 610 | 44,000 | 0.20 | Dimer | 26 |
| [symbol] mCherry | 587 | 610 | 72,000 | 0.22 | Monomer | 47 |
| .HcRed1 | 588 | 618 | 20,000 | 0.015 | Dimer | 1 |
| [symbol] mRaspberry | 598 | 625 | 86,000 | 0.15 | Monomer | 38 |
| .HcRed-Tandem | 590 | 637 | 160,000 | 0.04 | Monomer | 19 |
| .mPlum | 590 | 649 | 41,000 | 0.10 | Monomer | 12 |
| [symbol] AQ143 | 595 | 655 | 90,000 | 0.04 | Tetramer | 11 |

*Weak Dimer

### C. Recombinant Microorganism Compositions

Compositions comprising the microorganism, including bacteria, yeasts or fungi, selected using the methods described above are provided. The compositions may include a pharmaceutically acceptable carrier, diluent and/or excipient, in some aspects. The composition may include more than one microorganism, including bacteria, yeasts or fungi, isolate. The compositions may be formulated for delivery in food, water, via oral gavage, via an aerosol or sprayable product, drops, powder freeze-dried, extract, pill, suppository, and/or via enema, in specific aspects. In particular aspects, a L. reuteri composition is formulated to target a region in the gastrointestinal tract, including any portion of the gastrointestinal tract. The L. reuteri composition can be formulated to be delivered to any portion of the gastrointestinal tract. The composition can be formulated as a frozen composition, e.g., flash frozen, dried or lyophilized for storage and/or transport

The recombinant microorganism compositions encompassed herein may be administered in a variety of ways known or available to those skilled in the art. The strains or recombinant microorganism compositions may be administered in the form of a pharmaceutical, nutraceutical, added to food and/or water, and/or provided in aerosolized or sprayable form for administration by inhalation. In addition, the strains and recombinant microorganism compositions described herein may be provided as liquid suspensions, lyophilized or freeze dried powders or frozen concentrates for addition to target regions other than a subject, as examples.

In some aspects, the recombinant microorganisms are preferably not freeze- or spray-dried in a ready-to-use product, because the microorganisms preferably should be as robust as possible when entering the intestines in order to successfully compete with the pro-inflammatory part of the microflora already present in the intestines. However, some strains may be sufficiently robust and/or some protocols sufficiently gentle to allow spray- or freeze-dried probiotic microorganisms in a ready-to-use product.

In particular aspects, the recombinant microorganism or composition thereof are formulated to target a region in the gastrointestinal tract, including any portion of the gastrointestinal tract. In one aspect of any of the above methods involving administration of a recombinant microorganism composition, the recombinant microorganism composition is directly or indirectly delivered to the digestive tract of the subject. In some aspects, the recombinant microorganism delivered to the gut, or gastrointestinal tract, of the subject secretes one or more biologically active peptides in situ in the gastrointestinal tract of the subject. The compositions comprising the recombinant microorganism encompassed herein may be administered in a variety of ways known or available to those skilled in the art. In one aspect, the compositions comprising the recombinant microorganism are administered to the subject by a route selected from the group consisting of oral (e.g., by a pill, spray, capsule, aerosol, lozenge, bolus, tablet, sachet, liquid, suspension, or emulsion), rectal (e.g., by a suppository, enema, foam, suspension, or ointment), nasal, and via naso/oro-gastric gavage. While the compositions of the present disclosure may be formulated for oral administration, rectal, or naso/oro-gastric gavage, other routes of administration can be employed, however, including, but not limited to, mucosal and vaginal.

In one aspect, the recombinant microorganism composition is delivered to the subject in a form of a liquid, foam, cream, spray, powder, or gel. In one aspect, the recombinant microorganism composition comprises a buffering agent (e.g., sodium bicarbonate, infant formula or sterilized human milk, or other agents which allow bacteria to survive and grow (e.g., survive in the acidic environment of the stomach and to grow in the intestinal environment), along with preservatives, stabilizers, binders, compaction agents, lubricants, dispersion enhancers, disintegration agents, antioxidants, flavoring agents, sweeteners, and coloring agents.

In addition, the recombinant microorganism composition can administered alone or in combination with a carrier, such as a pharmaceutically acceptable carrier or a biocompatible scaffold. In some aspects, formulations of the composition comprise an ingestible carrier, which may be a pharmaceutically acceptable carrier such as a capsule, tablet or powder. In specific aspects, the ingestible carrier is a food product such as acidified milk, yogurt (frozen or non-frozen), milk powder, milk concentrate, cheese spreads, dressings and/or beverages. In some aspects, the composition further comprises a protein and/or peptide, in particular proteins and/or peptides that are rich in glutamine/glutamate, a lipid, a carbohydrate, a vitamin, mineral and/or trace element, for example. The formulation of the composition may further comprise an adjuvant, a drug, a biological compound, or a mixture thereof. The formulation of the composition may be a food stuff or a medicament, in certain aspects.

Additional formulations of the composition that are suitable for other modes of administration include oral formulations. Oral formulations include such normally employed excipients such as, for example, pharmaceutical grades of mannitol, lactose, starch, magnesium stearate, sodium saccharine, cellulose, magnesium carbonate and the like. These formulations take the form of solutions, suppositories, suspensions, tablets, pills, capsules, sustained release formulations or powders and contain about 10% to about 95% of active ingredient, preferably about 25% to about 70%. In some aspects, the compositions are formulated for oral administration. Oral administration may be achieved using a chewable formulation, a dissolving formulation, an encapsulated/coated formulation, a multi-layered lozenge (to separate active ingredients and/or active ingredients and excipients), a slow release/timed release formulation, or other suitable formulations known to persons skilled in the art. Although the word “tablet” is used herein, the formulation may take a variety of physical forms that may commonly be referred to by other terms, such as lozenge, pill, capsule, or the like.

In one aspect, the disclosed compositions are formulated as a capsule. The capsule may be a hollow, generally cylindrical capsule formed from various substances, such as gelatin, cellulose, carbohydrate or the like

In another aspect, the disclosed compositions are formulated as a suppository. The suppository may include but is not limited to the bacteria and one or more carriers, such as polyethylene glycol, acacia, acetylated monoglycerides, carnauba wax, cellulose acetate phthalate, corn starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monoleate, sucrose talc, titanium dioxide, white wax and coloring agents.

In some aspects, the disclosed compositions are prepared as a tablet. The tablet may include the bacteria and one or more tableting agents, such as dibasic calcium phosphate, stearic acid, croscarmellose, silica, cellulose and cellulose coating. The tablets may be formed using a direct compression process, though those skilled in the art will appreciate that various techniques may be used to form the tablets.

In particular aspects, the recombinant microorganism compositions of the present disclosure may be prepared as a powder that is intended to be dissolved in a liquid, such as water, milk, juice, and yogurt. It is understood that individual liquids may be mixed together where appropriate. For example, the recombinant microorganism formulation may be combined with fruit juice and yogurt or milk and yogurt to make probiotic yogurt shakes. The recombinant microorganism compositions may also be combined with milk and ice cream to make probiotic milk shakes. Flavorings for the recombinant microorganism liquid formulations contemplated in the disclosure are known to those of ordinary skill in the art.

The formulation of the compositions may be provided in food and/or beverage, in specific aspects. Particular examples include dairy products and/or probiotic-fortified foods. However, tablets, capsules, gums, or powders containing the bacteria in freeze-dried form are also available. In specific aspects, the L. reuteri is provided in a pill or in yogurt, for example. The formulation may be provided in infant formula and/or cereal (infant or otherwise), in some aspects.

In specific aspects, the compositions comprising the recombinant microorganism and related methods of the present disclosure utilize particular formulation methods. In some examples, the carrier may comprise a disintegrant, a glidant, and/or a lubricant, such as is described in U.S. Pat. No. 9,084,434, for example, to facilitate having a greater shelf life and/or half-life of the formulation. The disintegrant may be any suitable disintegrant such as, for example, a disintegrant selected from the group consisting of sodium croscarmellose, crospovidone, gellan gum, hydroxypropyl cellulose, starch, and sodium starch glycolate. The glidant may be any suitable glidant such as for example, a glidant selected from the group consisting of silicon dioxide, colloidal silicon dioxide, and talc. The lubricant may be any suitable lubricant such as for example, a lubricant selected from the group consisting of calcium stearate, magnesium stearate, stearic acid, sodium stearyl fumerate, and vegetable based fatty acids. In the composition and method of the present invention, the carrier is present in the composition in a range of approximately 30% w/w to approximately 98% w/w; this weight percentage is a cumulative weight percentage taking into consideration all ingredients present in the carrier. The composition of the present invention may be an oral dosage form, a powder that is mixed into a liquid, or a chewing gum. Where the composition is an oral dosage form, the oral dosage form may be selected from the group consisting of tablets, caplets, and capsules, wherein the tablets and caplets may be solid or chewable. Where the composition is a powder, it may be mixed into a liquid that is selected from the group consisting of water, milk, juice, and yogurt. Where the composition is a chewing gum, the gum may be soft gum or hard chewing gum tablets. By combining probiotic species with sugar alcohols, such as mannitol, sorbitol, alone or together with the additional sugar alcohol lactitol and/or a phytonutrient, such as oligomeric proanthocyanidins (OPC), the stability of probiotic formulations is increased under various storage conditions.

In particular aspects the formulation of the compositions comprising the recombinant microorganism comprises one or more excipients. Examples of excipients that may be used to formulate appropriate dosage forms include binders, disintegrants, lubricants, coatings, plasticizers, compression agents, wet granulation agents, and sweeteners, all of which are known to those of ordinary skill in the art to which the invention pertains. All of the following examples are provided by way of illustration and not limitation. Binders are used where appropriate to help the dosage form ingredients still together. Examples of binders include carbopol, povidone, and xanthan gum. Lubricants are generally always used in the manufacture of dosage forms by direct compression in order to prevent the compacted powder mass from sticking to the equipment during the tabletting or encapsulation process. Examples of lubricants include calcium stearate, magnesium stearate, stearic acid, sodium stearyl fumerate, and vegetable based fatty acids. Disintegrants aid in the break-up of the compacted mass when placed in a fluid environment. Examples of disintegrants include sodium croscarmellose, crospovidone, gellan gum, hydroxypropyl cellulose, starch, and sodium starch glycolate. Coatings are used to control the solubility of the drug. Examples of coatings include carrageenan, cellulose acetate phthalate, ethylcellulose, gellan gum, maltodextrin, methacrylates, methylcellulose, microcrystalline cellulose, and shellac. Plasticizers are used to control the release rate of the drug from the dosage form. Examples of plasticizers include citrate esters, dibutyl sebacate, diethyl phthalate, polyvinylacetate phthalate, and triacetin. Compression agents include calcium carbonate, dextrose, fructose, guar gum, honey, lactose, maltodextrin, maltose, mannitol, microcrystalline cellulose, molasses, sorbitol, starch, and sucrose. Wet granulation agents include calcium carbonate, lactose, maltodextrin, mannitol, microcrystalline cellulose, povidone, and starch. Sweeteners include aspartame, dextrose, fructose, honey, lactose, maltodextrin, maltose, mannitol, molasses, monoammonium glycyrrhizinate, sorbitol, sucralose, and sucrose. Excipients that are generally used in the manufacture of chewable tablets include by way of illustration and not limitation, dextrose, fructose, guar gum, lactose, maltodextrin, maltose, mannitol, microcrystalline cellulose, and sorbitol. As is evident from the foregoing list, many of the same ingredients may be used for various different purposes in various different dosage forms.

Aspects of the disclosure include one or more compositions or formulations thereof comprising a recombinant microorganism suitable for delivery to a subject in need thereof. A recombinant microorganism composition or formulation thereof may be provided as a formulation to the subject in any suitable form, and in particular aspects, the recombinant microorganisms of the composition or formulation are live, although in certain aspects, the recombinant microorganisms of the composition or formulation are dead. The recombinant microorganism compositions or formulations thereof of the disclosure can thus comprise, without limitation, e.g., bacterial, yeast, or fungi cells, including but not limited to conditionally lethal cells, inactivated cells, killed cells, spores (e.g., germination-competent spores), recombinant carrier strains, cell extract, and microorganism-derived products (natural or synthetic microorganism-derived products such as, e.g., microorganism antigens or metabolic products). One or several different recombinant microorganism inoculants can be administered simultaneously or sequentially (including administering at different times). Such microorganisms can be isolated from gastrointestinal (GI) microbiota and grown in culture. The present disclosure also comprises administering “bacterial analogues”, such as recombinant carrier strains expressing one or more heterologous genes derived from the relevant bacterial species. The use of such recombinant bacteria may allow the use of lower therapeutic amounts due to higher protein expression. In one aspect of any of the methods involving administration of a probiotic composition, the probiotic composition is reconstituted from a lyophilized preparation. In one aspect of any of the methods involving administration of a probiotic composition, said probiotic composition comprises a buffering agent to adjust pH to a suitable number, such as 7.0.

In some aspects, a recombinant microorganism composition or formulation thereof may comprise a therapeutically effective amount of one or more bacteria or yeast. As used here, a “therapeutically effective” amount of a bacterium or yeast describes an amount sufficient to be effective in treating a desired condition, for example, an immune system-mediated disorder. In some aspects, a therapeutically effective amount of isolated or purified populations of bacteria or yeast administered to a human will be at least, at most, or about 1×10³ colony forming units (CFU) of bacteria or yeast or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU (or any derivable range or value therein). In some aspects, a single dose will contain bacteria (such as a specific bacteria or species, genus, or family described herein) or yeast (such as a specific yeast or species, genus, or family described herein) present in an amount of least, at most, or about 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or more CFU (or any derivable or value range therein). In some aspects, a single dose will contain at least, at most, or about 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ CFU (or any derivable range or value therein) of total bacteria or yeast.

In some aspects, a therapeutically effective amount of each isolated or purified population of bacteria or yeast that is administered will be at least, at most, or about 1×10³ cells of bacteria or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ cells (or any derivable range or value therein). In some aspects, a single dose will contain bacteria (such as a specific bacteria or species, genus, or family described herein) or yeast (such as a specific yeast or species, genus, or family described herein) present in an amount of at least, at most, or about 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or more cells (or any derivable range or value therein). In some aspects, a single dose will contain at least, at most, or about 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ cells (or any derivable range or value therein) of total bacteria or yeast.

In particular aspects, each dose of the recombinant microorganism composition or formulation thereof may comprise a certain range of bacterial or yeast cells. In specific aspects, the recombinant microorganism composition or formulation thereof comprises a range of about 10³ cells to about 10¹³ cells. In certain aspects, each dose is in the range of about 10⁵ cells to about 10¹¹ cells, about 10⁵ to about 10¹⁰ cells, about 10⁷ cells to about 10¹⁰ cells, and so forth. In other aspects, each dose is in the range of about 5×10⁹ cells.

In particular aspects, the bacterial or yeast cells in each dose of the recombinant microorganism composition and/or the amount of one or more biologically active peptides produced and/or secreted by the bacterial or yeast cells is at least, at most, or about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 pg/kg, ng/kg, pg/kg, mg/kg, pg/day, ng/day, pg/day, or mg/day or any range derivable therein, of the one or more biologically active peptides. Furthermore, such doses can be administered at multiple times during a day, and/or on multiple days, weeks, or months.

In certain aspects, the effective dose of compositions comprising the recombinant microorganisms is one which can provide a blood level of the one or more biologically active peptides produced and/or secreted by the recombinant microorganisms of about 1 pM to 150 μM. In another aspect, the effective dose provides a blood level of about 1 pM to 100 μM; or about 1 pM to 50 μM; or about 1 pM to 20 μM; or about 1 pM to 10 μM; or about 1 pM to 1 μM; or about 100 pM to 900 nM; or about 200 pM to 800 nM; or about 300 pM to 700 nM; or about 400 pM to 600 nM; or about 500 pM to 500 nM; or about 600 pM to 400 nM; or about 700 pM to 300 nM; or about 800 pM to 200 nM; or about 900 pM to 100 nM (or any range derivable therein). In other aspects, the dose can provide the following blood level of the one or more biologically active peptides that results from administration of the recombinant microorganism or composition thereof to a subject: about, at least, at most, or about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 pM, nM, μM or any range derivable therein. In certain aspects, the one or more biologically active peptides produced and/or secreted by the recombinant microorganisms administered to a subject are metabolized in the body to a metabolite of the biologically active peptides, in which case the blood levels may refer to the amount of that metabolite. Alternatively, to the extent the one or more biologically active peptides produced and/or secreted by the recombinant microorganisms are not metabolized by a subject, the blood levels discussed herein may refer to the unmetabolized biologically active peptides.

It will be understood by those skilled in the art and made aware that dosage units of pg/kg, ng/kg, μg/kg, or mg/kg of body weight can be converted and expressed in comparable concentration units of pg/ml, ng/ml, μg/ml or pM, nM, μM, or mM (blood levels), such as 1 pM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

The one or more compositions of the recombinant microorganism can be administered alone or in combination with one or more additional therapeutic agents disclosed herein. The one or more additional therapeutic agents may include but are not limited to, for example: nonsteroidal anti-inflammatory drugs, one or more corticosteroids, one or more disease-modifying anti-rheumatic drugs, or one or more antibodies targeted to cytokines or cytokine receptors.

Administration “in combination with” one or more additional therapeutic agents includes both simultaneous (at the same time) and consecutive administration in any order. The compositions of the recombinant microorganism and one or more additional therapeutic agents can be administered in one composition, or simultaneously as two separate compositions, or sequentially. Administration can be chronic or intermittent, as deemed appropriate by the supervising practitioner, including in view of any change in any undesirable side effects.

An effective amount of the compositions comprising the recombinant microorganism or composition thereof may be provided to a subject in need thereof and may or may not be provided with one or more additional therapeutic agents. The additional therapeutic agents may be in the same composition as the recombinant microorganism or composition thereof, or the additional therapeutic agents may be in a different composition as the recombinant microorganism or composition thereof. The additional therapeutic agents may or may not be given to the subject at the same time as the recombinant microorganism or composition thereof. The additional therapeutic agents may assist in treating and/or preventing at least disorder that may be mediated by cells of the immune system and/or fibroblasts or fibroblast-like cells and/or the additional therapeutic agents may be useful for treating and/or preventing at least one symptom of another medical condition.

In some aspects, the recombinant microorganism or composition thereof is administered prior to the additional therapeutic agents. In some aspects, the recombinant microorganism or composition thereof is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) prior to the additional therapeutic agents. In some aspects, at least, at most, or about 1, 2, 3, 4, 5, 6, or 7 doses (or any derivable range therein) of the recombinant microorganism or composition thereof is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) prior to the additional therapeutic agents. In some aspects, the recombinant microorganism or composition thereof is administered after the additional therapeutic agents. In some aspects, the recombinant microorganism or composition thereof is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) after the additional therapeutic agents. In some aspects, at least, at most, or about 1, 2, 3, 4, 5, 6, or 7 doses (or any derivable range therein) of the recombinant microorganism or composition thereof is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) after the additional therapeutic agents.

In specific aspects, compositions comprising the additional therapeutic agents of the present disclosure utilize particular formulation methods. In some examples, the carrier may comprise a disintegrant, a glidant, and/or a lubricant, such as is described in U.S. Pat. No. 9,084,434, for example, to facilitate having a greater shelf life and/or half-life of the formulation. The disintegrant may be any suitable disintegrant such as, for example, a disintegrant selected from the group consisting of sodium croscarmellose, crospovidone, gellan gum, hydroxypropyl cellulose, starch, and sodium starch glycolate. The glidant may be any suitable glidant such as for example, a glidant selected from the group consisting of silicon dioxide, colloidal silicon dioxide, and talc. The lubricant may be any suitable lubricant such as for example, a lubricant selected from the group consisting of calcium stearate, magnesium stearate, stearic acid, sodium stearyl fumerate, and vegetable based fatty acids. In the composition and method of the present invention, the carrier is present in the composition in a range of approximately 30% w/w to approximately 98% w/w; this weight percentage is a cumulative weight percentage taking into consideration all ingredients present in the carrier. The composition of the present invention may be an oral dosage form, a powder that is mixed into a liquid, or a chewing gum. Where the composition is an oral dosage form, the oral dosage form may be selected from the group consisting of tablets, caplets, and capsules, wherein the tablets and caplets may be solid or chewable. Where the composition is a powder, it may be mixed into a liquid that is selected from the group consisting of water, milk, juice, and yogurt. Where the composition is a chewing gum, the gum may be soft gum or hard chewing gum tablets. By combining probiotic species with sugar alcohols, such as mannitol, sorbitol, alone or together with the additional sugar alcohol lactitol and/or a phytonutrient, such as oligomeric proanthocyanidins (OPC), the stability of probiotic formulations is increased under various storage conditions.

In particular aspects the formulation of the compositions comprising the additional therapeutic agents comprises one or more excipients. Examples of excipients that may be used to formulate appropriate dosage forms include binders, disintegrants, lubricants, coatings, plasticizers, compression agents, wet granulation agents, and sweeteners, all of which are known to those of ordinary skill in the art to which the invention pertains. All of the following examples are provided by way of illustration and not limitation. Binders are used where appropriate to help the dosage form ingredients still together. Examples of binders include carbopol, povidone, and xanthan gum. Lubricants are generally always used in the manufacture of dosage forms by direct compression in order to prevent the compacted powder mass from sticking to the equipment during the tabletting or encapsulation process. Examples of lubricants include calcium stearate, magnesium stearate, stearic acid, sodium stearyl fumerate, and vegetable based fatty acids. Disintegrants aid in the break-up of the compacted mass when placed in a fluid environment. Examples of disintegrants include sodium croscarmellose, crospovidone, gellan gum, hydroxypropyl cellulose, starch, and sodium starch glycolate. Coatings are used to control the solubility of the drug. Examples of coatings include carrageenan, cellulose acetate phthalate, ethylcellulose, gellan gum, maltodextrin, methacrylates, methylcellulose, microcrystalline cellulose, and shellac. Plasticizers are used to control the release rate of the drug from the dosage form. Examples of plasticizers include citrate esters, dibutyl sebacate, diethyl phthalate, polyvinylacetate phthalate, and triacetin. Compression agents include calcium carbonate, dextrose, fructose, guar gum, honey, lactose, maltodextrin, maltose, mannitol, microcrystalline cellulose, molasses, sorbitol, starch, and sucrose. Wet granulation agents include calcium carbonate, lactose, maltodextrin, mannitol, microcrystalline cellulose, povidone, and starch. Sweeteners include aspartame, dextrose, fructose, honey, lactose, maltodextrin, maltose, mannitol, molasses, monoammonium glycyrrhizinate, sorbitol, sucralose, and sucrose. Excipients that are generally used in the manufacture of chewable tablets include by way of illustration and not limitation, dextrose, fructose, guar gum, lactose, maltodextrin, maltose, mannitol, microcrystalline cellulose, and sorbitol. As is evident from the foregoing list, many of the same ingredients may be used for various different purposes in various different dosage forms.

The additional therapeutic agents of the disclosure may be administered by the same route of administration as the recombinant microorganism compositions or by different routes of administration. In some aspects, the recombinant microorganism or a composition thereof and/or compositions comprising additional therapeutic agents are administered intravenously, intramuscularly, subcutaneously, topically, orally, transdermally, intraperitoneally, intraorbitally, by implantation, by inhalation, intrathecally, intraventricularly, or intranasally. In some aspects, the microbial composition is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the subject, the subject's clinical history and response to the treatment, and the discretion of the attending physician.

The treatments with the therapeutic agents may include various “unit doses.” Unit dose is defined as containing a predetermined-quantity of the therapeutic composition calculated to produce the desired responses discussed above in association with its administration, i.e., the appropriate route and regimen. The quantity to be administered, and the particular route and formulation, is within the skill of determination of those in the clinical arts and depends on the result and/or protection desired. A unit dose need not be administered as a single injection but may comprise continuous infusion over a set period of time. In some aspects, a unit dose comprises a single administrable dose.

The quantity of additional therapeutic agents to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of at least, at most, or about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the additional therapeutic agents in compositions comprising the additional therapeutic agents is one which can provide a blood level of about 1 μM to 150 μM of the additional therapeutic agents. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

Typically, the recombinant microorganism or a composition thereof and/or compositions comprising additional therapeutic agents will be administered in a manner compatible with the dosage formulation, and in such amount as will be therapeutically or prophylactically effective for the subject being treated. Precise amounts of the recombinant microorganism or a composition thereof and/or compositions comprising additional therapeutic agents also depend on the judgment of the practitioner and are peculiar to each individual. Suitable regimes for initial administration and boosters are also variable, but are typified by an initial administration followed by subsequent administrations. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

The recombinant microorganism or a composition thereof and/or compositions comprising additional therapeutic agents will be pharmaceutically acceptable or pharmacologically acceptable. The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

### V. Methods of Treatment and Use of the Disclosure

Methods of the disclosure allow for the treatment or prevention a medical condition by administering a therapeutically effective amount of a recombinant microorganism or composition thereof, wherein the recombinant microorganism is capable of producing a therapeutically effective amount of the one or more biologically active peptides to treat or prevent one or more diseases or conditions including but not limited to one or more diseases or conditions that may be mediated by cells of the immune system and/or fibroblasts or fibroblast-like cells. The disclosure encompasses methods and compositions for in situ expression of one or more biologically active peptides by a recombinant microorganism in the gut, or gastrointestinal tract, to treat or prevent one or more diseases or conditions including but not limited to one or more diseases or conditions that may be mediated by cells of the immune system and/or fibroblasts or fibroblast-like cells.

In some aspects, the disease or condition to be treated or prevented by administering a therapeutically effective amount of a recombinant microorganism or composition thereof comprises type 2 diabetes, HIV, cancer, short bowel syndrome, or hepatitis (including hepatitis B and C). Tumors for which the present treatment methods are useful include any malignant cell type, such as those found in a solid tumor or a hematological tumor. In aspects wherein the individual has cancer, the cancer may be primary, metastatic, resistant to therapy, and so forth. In specific aspects, the present therapy is useful for individuals with cancers that have been clinically indicated to be subject to immune cell regulation, including multiple types of solid tumors (melanoma, colon, lung, breast, and head and neck cancers), for example. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

In some aspects, the disease or condition to be treated or prevented by administering a therapeutically effective amount of a recombinant microorganism or composition comprises one or more diseases or conditions that may be mediated by cells of the immune system and/or fibroblasts or fibroblast-like cells, such as an immune system disorder. As used herein, “immune system-mediated disorder” or “immune system disorder” refers to any disease or medical condition that is mediated by autoreactive (self-reactive) immune cells. In some aspects, an antigen provokes a T-cell mediated immune response, and the immune system-mediated disorder is a T-cell mediated disorder. In some aspects, autoreactive B cells differentiate upon repetitive autoantigen stimulation into class-switched memory B cells, and the immune system-mediated disorder is a B-cell mediated disorder. In some aspects, cells including fibroblasts and/or fibroblast-like cells, which can initiate inflammation by coordinating recruitment of immunocompetent cells, act as non-immune cell targets for treating or preventing immune system disorders such as rheumatoid arthritis.

Immune system-mediated disorders include but are not limited to acne vulgaris, allergy, food allergy, atherosclerosis, asthma, allergic asthma, autoimmune diseases, autoinflammatory diseases, celiac disease, chronic inflammatory demyelinating polyneuropathy, chronic prostatitis, colitis, diverticulitis, familial Mediterranean fever, glomerulonephritis, Graves' disease, Guillain-Barre syndrome, Hashimoto's thyroiditis, hidradenitis suppurativa, hypersensitivities, inflammatory bowel diseases, interstitial cystitis, leukocyte defects, lichen planus, mast-cell activation syndrome, mastocytosis, multiple sclerosis, myasthenia gravis, myopathy, otitis, pelvic inflammatory disease, pneumonia, psoriasis, reperfusion injury, rheumatic fever, rheumatoid arthritis, rhinitis, sarcoidosis, Sjorgen's syndrome, systemic lupus erythematosus, thyroiditis, transplant rejection (e.g., graft versus host disease), type I diabetes, vasculitis, and uveitis.

The antigen that provokes an immune response can be any antigen, such as, but not limited to allergens (including food allergens), allo-antigens, self-antigens, auto-antigens, and therapeutic molecules or antigens that induce an immune response. In some aspects, the antigen is involved in the induction of immune system-mediated disorders. In some aspects, the antigen is involved in the induction of acne vulgaris, allergy, food allergy, atherosclerosis, asthma, allergic asthma, autoimmune diseases, autoinflammatory diseases, celiac disease, chronic inflammatory demyelinating polyneuropathy, chronic prostatitis, colitis, diverticulitis, familial Mediterranean fever, glomerulonephritis, Graves' disease, Guillain-Barre syndrome, Hashimoto's thyroiditis, hidradenitis suppurativa, hypersensitivities, inflammatory bowel diseases, interstitial cystitis, leukocyte defects, lichen planus, mast-cell activation syndrome, mastocytosis, multiple sclerosis, myasthenia gravis, myopathy, otitis, pelvic inflammatory disease, pneumonia, psoriasis, reperfusion injury, rheumatic fever, rheumatoid arthritis, rhinitis, sarcoidosis, Sjorgen's syndrome, reperfusion injury, rheumatic fever, rheumatoid arthritis, rhinitis, sarcoidosis, systemic lupus erythematosus, thyroiditis, transplant rejection (e.g., graft versus host disease), type I diabetes, vasculitis, or uveitis.

In specific aspects, the subject has one or more diseases or conditions that may be mediated by cells of the immune system and/or fibroblasts or fibroblast-like cells, such as an immune system-mediated disorder, and such a disorder may have any cause, including deprivation, genetic and metabolic diseases, infectious diseases, nutritional factors, physical trauma, and toxic and environmental factors, for example; the disorder may have two or more causes. In particular aspects, a subject that is the subject for methods and compositions of the disclosure has one or more diseases or conditions that may be mediated by cells of the immune system and/or fibroblasts or fibroblast-like cells, for example, an immune system-mediated disorder, in which at least one symptom is inflammation. Other symptoms include but are not limited to fatigue, achy muscles, swelling and redness, low-grade fever, hair loss, and skin rashes.

A subject having one or more diseases or conditions that may be mediated by cells of the immune system and/or fibroblasts or fibroblast-like cells is provided a therapeutically effective amount of a recombinant microorganism or composition thereof to treat or prevent the disease or condition, which may have as a symptom inflammation. As used herein, the term “therapeutically effective amount” is synonymous with “effective amount,” “therapeutically effective dose,” and/or “effective dose,” and refers to an amount of an agent sufficient to produce a desired result or exert a desired influence on the particular condition being treated. In some aspects, a therapeutically effective amount is an amount sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. For instance, in some aspects, the effective amount refers to the amount of a recombinant microorganism or composition thereof that can treat or prevent inflammation and/or the one or more diseases or conditions that may be mediated by cells of the immune system and/or fibroblasts or fibroblast-like cells in a subject. The effective amount may vary depending on the organism or individual treated. The appropriate effective amount to be administered for a particular application of the disclosed methods can be determined by those skilled in the art, using the guidance provided herein. As used herein, the terms “treatment,” “treat,” or “treating” refers to intervention in an attempt to alter the natural course of the subject being treated, and may be performed either for prophylaxis or during the course of pathology of a disease or condition. Treatment may serve to accomplish one or more of various desired outcomes, including, for example, preventing occurrence or recurrence of disease, alleviation or reduction in severity of symptoms, and diminishment of any direct or indirect pathological consequences of the disease, preventing disease spread, lowering the rate of disease progression, amelioration or palliation of the disease state, and remission or improved prognosis.

In some aspects, there may be a regimen having a first phase for an initial treatment, which may last for days, weeks, months, or years, and then another phase for maintenance, which may last for weeks, months, or years. Such a treatment may be administered one or more times a day, including at least, at most, or about 1, 2, 3, or more times per day, for a period sufficient to stabilize the gut flora. In other aspects, in a maintenance phase, a subject is provided a lesser amount of the recombinant microorganism or composition thereof and/or fewer administrations than the initial treatment phase. The duration of a treatment regimen may be dependent on each individual patient and the stage of the medical condition. In some aspects, a continued treatment for a certain period of time occurs until a detectable improvement in inflammation and/or the one or more diseases or conditions that may be mediated by cells of the immune system and/or fibroblasts or fibroblast-like cells is observed. In some aspects, the improvements are maintained by additional treatment, although the additional treatment may be reduced in frequency and/or volume.

In a particular form of the disclosure, an initial treatment regimen comprising a therapeutically effective amount of a recombinant microorganism or composition thereof may comprise of at least, at most, or about 10⁵, 10⁶, 10⁷, 10⁸, 10⁹, 10¹⁰, 1×10¹², 1×10¹³, 1×10¹⁴, 1×10¹⁵ or greater than 1×10¹⁵ cells per dose or any range or value derivable therein. In some aspects, an initial treatment regimen comprising a therapeutically effective amount of a recombinant microorganism or composition thereof may comprise at least, at most, or about 5×10⁹ cells per dose. In some aspects, the cells in an initial treatment regimen comprising a therapeutically effective amount of a recombinant microorganism or composition thereof and/or the amount of one or more biologically active peptides produced and/or secreted by the recombinant microorganism is at least, at most, or about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 pg/kg, ng/kg, μg/kg, mg/kg, pg/day, ng/day, μg/day, or mg/day or any range derivable therein, of the one or more biologically active peptides. In certain aspects, the therapeutically effective amount of compositions comprising the recombinant microorganisms is one which can provide a blood level of the one or more biologically active peptides produced and/or secreted by the recombinant microorganisms of about 1 pM to 150 μM. In other aspects, the dose can provide the following blood level of the one or more biologically active peptides that results from administration of the recombinant microorganism or composition thereof to a subject: at least, at most, or about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 pM, nM, μM or any range derivable therein.

In a particular form of the disclosure, an initial treatment regimen may comprise an effective amount of a recombinant microorganism or composition thereof and one or more additional therapeutics. In some aspects, the one or more additional therapeutics may comprise at least, at most, or about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, pg/day, or mg/day or any range or value derivable therein.

In a particular form of the disclosure, an initial treatment regimen comprises an effective amount of a recombinant microorganism or composition, which may comprise at least, at most, or about 10⁵, 10⁶, 10⁷, 10⁸, 10⁹, 10¹⁰, 1×10¹², 1×10¹³, 1×10¹⁴, 1×10¹⁵ or greater than 1×10¹⁵ cells per dose or any range or value derivable therein, for example, 5×10⁹ cells per dose, such that a therapeutically effective amount of a recombinant microorganism or composition thereof and/or the amount of one or more biologically active peptides produced and/or secreted by the recombinant microorganism is at least, at most, or about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 pg/kg, ng/kg, μg/kg, mg/kg, pg/day, ng/day, μg/day, or mg/day or any range derivable therein, and at least an effective amount of one or more additional therapeutics, which may comprise at least, at most, or about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range or value derivable therein.

In some aspects, disclosed herein are methods of treating or preventing one or more immune system-mediated disorders, the method comprising administering to a subject in need thereof a therapeutically effective amount of the recombinant microorganism or a composition, wherein the recombinant microorganism is capable of producing a therapeutically effective amount of the one or more biologically active peptides to treat or prevent the one or more immune system-mediated disorders. In some aspects, the immune system-mediated disorder is rheumatoid arthritis. In some aspects, the immune system-mediated disorder is psoriasis.

In specific aspects, a treatment regimen is for a subject with one or more diseases or conditions that may be mediated by cells of the immune system and/or fibroblasts or fibroblast-like cells, such as an immune system-mediated disorder, and the one or more diseases or conditions may have as a symptom inflammation. The immune system-mediated disorder and/or inflammation may be caused at least in part by proliferation and/or activation of CCR7⁻ effector memory T-cells and/or IgD⁻ class-switched memory B-cells, and the one or more biologically active peptides secreted by the recombinant microorganism can inhibit the proliferation and/or activation of CCR7⁻ effector memory T-cells and/or IgD⁻ class-switched memory B-cells to reduce inflammation and/or treat or prevent the immune system-mediated disorder. In some aspects, the CCR7⁻ effector memory T-cells and/or IgD⁻ class-switched memory B-cells are specific for autoantigens. The immune system-mediated disorder and/or inflammation may also be caused at least in part by proliferation and/or activation of non-immune cells including fibroblasts and/or fibroblast-like cells, such as FLS, and the one or more biologically active peptides secreted by the recombinant microorganism can inhibit the proliferation, invasiveness, and secretion of cytokines, chemokines, proteases and growth factors by fibroblasts and/or fibroblast-like cells, such as FLS, to reduce inflammation and/or treat or prevent the immune system-mediated disorder. In some aspects, the one or more biologically active peptides secreted by the recombinant microorganism can also treat or prevent other diseases in which fibroblasts are involved, including but not limited to immune-checkpoint inhibitor-induced rheumatoid arthritis, scleroderma, lung fibrosis, and diabetic skin ulcers.

In particular aspects, targeting immune cells via administration of a recombinant microorganism or composition thereof is useful to treat one or more diseases or conditions that may be mediated by cells of the immune system and/or fibroblasts or fibroblast-like cells, such as one or more immune system-mediated disorders. In particular aspects, one or more biologically active peptides produced and secreted by a recombinant microorganism inhibits the proliferation and/or activation of immune cells, such as CCR7⁻ T_{EM} cells and/or IgD⁻ class-switched memory B-cells, thereby reducing inflammation caused by immune cells, for example. Thus, in some aspects, targeting T_{EM} cells and/or class-switched memory B-cells with one or more biologically active peptides can reduce inflammation and/or treat or prevent immune-system related disorders. In some aspects, the one or more biologically active peptides produced and secreted by a recombinant microorganism to inhibit the proliferation and/or activation of immune cells, such as CCR7⁻ T_{EM} cells and/or IgD⁻ class-switched memory B-cells, are immunomodulatory peptides, which can be immune cell ion channel blockers. In some aspects, the proliferation and/or activation of immune cells, such as CCR7⁻ T_{EM} cells and/or IgD⁻ class-switched memory B-cells, is inhibited by blocking ion channels, such as potassium channels like Kv1.3 potassium channels, present in the plasma membrane of the immune cells. Thus, in some aspects, inhibiting T_{EM} cells and/or class-switched memory B-cells by blocking Kv1.3 potassium channels with one or more potassium channel blockers can treat or prevent immune-system related disorders and/or reduce inflammation associated therewith.

In particular aspects, targeting cells including fibroblasts and/or fibroblast-like cells, such as FLS, via administration of a recombinant microorganism or composition thereof is useful to treat immune system-mediated disorders. In particular aspects, one or more biologically active peptides produced and secreted by a recombinant microorganism inhibits the proliferation, invasiveness, and secretion of cytokines, chemokines, proteases and growth factors by fibroblasts and/or fibroblast-like cells, such as FLS, thereby reducing inflammation, for example. Thus, in some aspects, targeting fibroblasts and/or fibroblast-like cells, such as FLS, with one or more biologically active peptides can reduce inflammation and/or treat or prevent immune-system related disorders. In some aspects, the one or more biologically active peptides produced and secreted by a recombinant microorganism to inhibit the proliferation, invasiveness, and secretion of cytokines, chemokines, proteases and growth factors by fibroblasts and/or fibroblast-like cells, such as FLS, are ion channel blockers. In some aspects, the proliferation, invasiveness, and secretion of cytokines, chemokines, proteases and growth factors by fibroblasts and/or fibroblast-like cells, such as FLS, is inhibited by blocking ion channels, such as potassium channels like KCa1.1 potassium channels, present in the plasma membrane of the fibroblasts and/or fibroblast-like cells, such as FLS. Thus, in some aspects, inhibiting fibroblasts and/or fibroblast-like cells, such as FLS, by blocking KCa1.1 potassium channels with one or more potassium channel blockers can treat or prevent immune-system related disorders and/or reduce inflammation associated therewith.

The subject may have or may be at risk of having one or more diseases or conditions that may be mediated by cells of the immune system and/or fibroblasts or fibroblast-like cells. The subject may have or may be at risk of having one or more of acne vulgaris, allergy, food allergy, atherosclerosis, asthma, allergic asthma, autoimmune diseases, autoinflammatory diseases, celiac disease, chronic inflammatory demyelinating polyneuropathy, chronic prostatitis, colitis, diverticulitis, familial Mediterranean fever, glomerulonephritis, Graves' disease, Guillain-Barre syndrome, Hashimoto's thyroiditis, hidradenitis suppurativa, hypersensitivities, inflammatory bowel diseases, interstitial cystitis, leukocyte defects, lichen planus, mast-cell activation syndrome, mastocytosis, multiple sclerosis, myasthenia gravis, myopathy, otitis, pelvic inflammatory disease, pneumonia, psoriasis, reperfusion injury, rheumatic fever, rheumatoid arthritis, rhinitis, sarcoidosis, Sjorgen's syndrome, reperfusion injury, rheumatic fever, rheumatoid arthritis, rhinitis, sarcoidosis, systemic lupus erythematosus, thyroiditis, transplant rejection (e.g., graft versus host disease), type I diabetes, vasculitis, or uveitis. In some aspects, the subject has or is at risk of having rheumatoid arthritis. In some aspects, the subject has or is at risk of having psoriasis.

Methods of the disclosure include methods of treating or preventing one or more diseases or conditions that may be mediated by cells of the immune system and/or fibroblasts or fibroblast-like cells which may have as a symptom inflammation. In some aspects, the subject is at a higher risk than an average person in the general population. In some aspects, the one or more diseases or conditions that may be mediated by cells of the immune system and/or fibroblasts or fibroblast-like cells poses a greater risk to the health or life of the subject than such a condition would pose to an average person in the general population. In some aspects, the method is employed for a subject where it is uncertain whether or not risk of developing one or more diseases or conditions that may be mediated by cells of the immune system and/or fibroblasts or fibroblast-like cells is increased, whereas in other aspects the method is employed for a subject where it is known that the risk of developing one or more diseases or conditions that may be mediated by cells of the immune system and/or fibroblasts or fibroblast-like cells is increased. In other aspects, it has been determined that the risk of developing one or more diseases or conditions that may be mediated by cells of the immune system and/or fibroblasts or fibroblast-like cells is increased for the subject, but the methods of the disclosure are still employed as a routine matter or in the general therapeutic interest of the subject.

In some aspects, the subject does not exhibit one or more diseases or conditions that may be mediated by cells of the immune system and/or fibroblasts or fibroblast-like cells and/or inflammation when a recombinant organism or composition thereof is administered. In some aspects, the subject has been diagnosed with one or more diseases or conditions that may be mediated by cells of the immune system and/or fibroblasts or fibroblast-like cells and/or inflammation. In some aspects, the recombinant organism or composition thereof is administered after the subject has been diagnosed with one or more diseases or conditions that may be mediated by cells of the immune system and/or fibroblasts or fibroblast-like cells and/or inflammation, and the recombinant organism or composition thereof may be administered to the subject until the subject is no longer symptomatic.

In specific aspects, a subject is provided a therapy or preventative for one or more diseases or conditions that may be mediated by cells of the immune system and/or fibroblasts or fibroblast-like cells in addition to the compositions encompassed herein. For example, they may be provided one or more of the following in addition to and/or in combination with the recombinant organism or composition thereof: nonsteroidal anti-inflammatory drugs, one or more corticosteroids, one or more disease-modifying anti-rheumatic drugs, or one or more antibodies targeted to cytokines or cytokine receptors.

### VI. Kits

Kits are also included as part of the disclosure. Kits for implementing methods of the invention described herein are specifically contemplated. In some aspects, there are kits for treating diseases or conditions that may be mediated by cells of the immune system and/or fibroblasts or fibroblast-like cells in a subject and/or preventing onset of at least one symptom of diseases or conditions that may be mediated by cells of the immune system and/or fibroblasts or fibroblast-like cells in a subject. The kit may be provided for treatment and/or prevention of immune-system mediated disorders, for example, T-cell- and/or B-cell-mediated disorders, in a subject having inflammation due in part the activation and/or proliferation of immune cells, for example, effector memory T-cells and/or class-switched memory B-cells. The kit may be provided for treatment and/or prevention of disorders in a subject having inflammation due in part the activation and/or proliferation of fibroblasts and/or fibroblast-like cells, for example, FLS.

The container means of the kits will generally include at least one vial, test tube, flask, bottle, syringe or other container means, into which a recombinant microorganism component (including L. reuteri) and/or a therapeutic agent component may be placed, and preferably, suitably aliquoted. Where there are other components in the kit other than a recombinant microorganism component (including L. reuteri) and/or a therapeutic agent component, the kit also will generally contain a second, third, or other additional container into which the additional components (such as another recombinant microorganism or therapeutic agent) may be separately placed. However, various combinations of components may be comprised in a vial. The kits of the present disclosure also will typically include a means for containing the compositions, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as a stab or as a dried powder(s). When reagents and/or components are provided as a dry powder, the powder can be reconstituted by the addition of a suitable solvent. It is envisioned that the solvent may also be provided in another container means. The components of the kits may be packaged either in aqueous media or in lyophilized form.

A kit will also include instructions for employing the kit components as well the use of any other reagent not included in the kit. Instructions may include variations that can be implemented.

### EXAMPLES

The following examples are included to demonstrate preferred aspects of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the disclosure.

### Example 1

### Immune Cell Kv1.3 Potassium Channels as Targets of Recombinant Microorganism-Derived Therapeutic for Treatment of Immune System-Mediated Disorders

1. Selection of Kv1.3 K⁺ Channels as Therapeutic Target on CCR7⁻ T_{EM} Cells

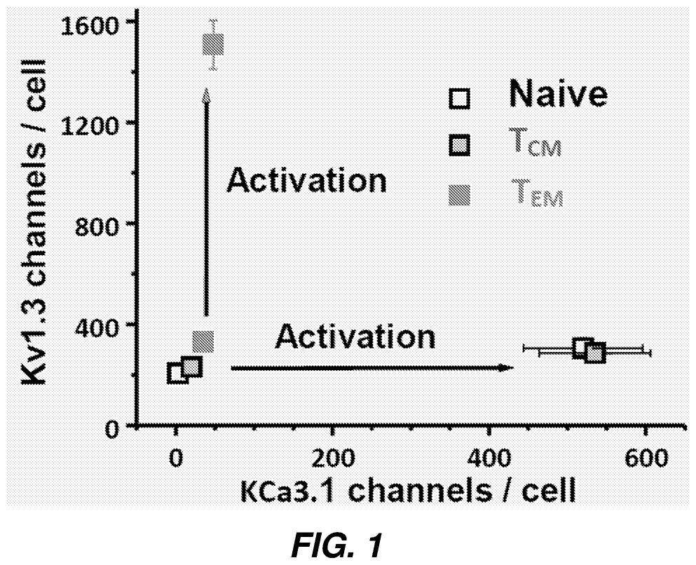

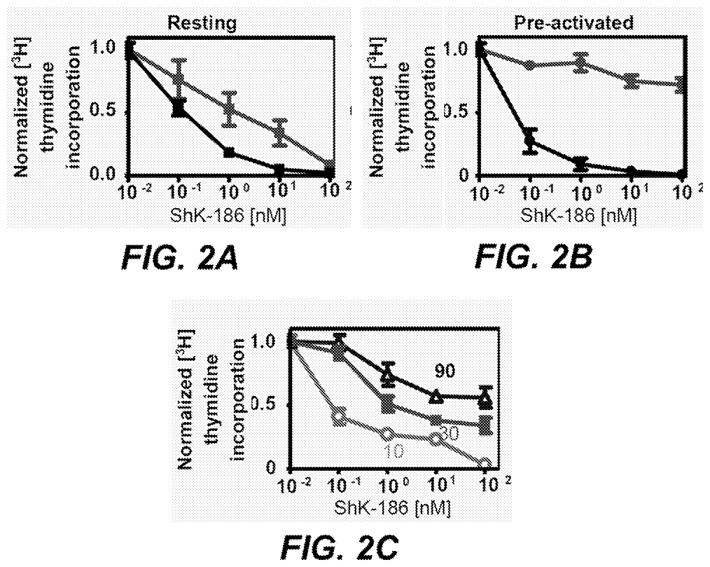

The activation of CCR7⁻ T_{EM} cells induces the upregulation of the Kv1.3 K⁺ channel (FIG. 1)^{8,9}. In contrast, CCR7⁺ naïve and central memory T (T_{CM}) cells upregulate a different K⁺ channel (KCa3.1) upon activation^{8,9,35}. The inventors therefore designed selective Kv1.3 blockers and assessed the sensitivity of the different T-cell subsets to Kv1.3 block. Kv1.3 blockers preferentially inhibit human CCR7⁻ T_{EM} cells over CCR7⁺ naïve and T_{CM} cells (FIG. 2)^{6,8,9,36}. The selective Kv1.3 blocker ShK-186 was 10-fold more effective in suppressing [³H]-thymidine incorporation, an indicator of cell proliferation, by T_{EM} cells compared with naïve/T_{CM} cells from patients with type 1 diabetes or RA (FIG. 2A), but when cells were activated overnight, rested and re-stimulated, T_{EM} cells remained exquisitely sensitive to the Kv1.3 blocker at pharmacologically relevant concentrations, while naïve/T_{CM} cells were resistant (FIG. 2B)⁸. Without wishing to be bound by theory, this “escape” by naïve/T_{CM} effectors may be due to up-regulation of the calcium-activated KCa3.1 channel that regulates membrane potential and Ca²⁺ signaling in naïve/T_{CM} effectors in place of Kv1.38. The significance of this finding is that in some aspects Kv1.3 blockers have an advantage over current immunotherapies because naïve and long-lived T_{CM} cells (the main memory pool) would escape inhibition while T_{EM} cells would be targeted.

While global suppression of all T_{EM} cells pose a problem in some aspects, the inventors found that T_{EM} cells escaped Kv1.3 blockers when the strength of antigenic stimulus increased (FIG. 2C)⁸, suggesting that in some aspects, high-affinity T_{EM} cells specific for pathogens and vaccine antigens overcomes Kv1.3 blockade while moderate-to-weak affinity T_{EM} cells specific for autoantigens remains suppressed. Indeed, whereas Kv1.3 blockers reduce incidence and severity of animal models of autoimmunity^{6,8,33,34,36,37}, they do not affect the clearance of influenza or chlamydia infections in rats⁸ or induce the reactivation of latent CMV in non-human primates³⁸. ShK-186 (dalazatide)³⁹, a Kv1.3 blocker that has successfully completed Phase 1A and 1B clinical trials in healthy volunteers and in patients with active plaque psoriasis, displayed both safety and efficacy after repeated systemic injections¹⁸.

### 2. Design of Selective Kv1.3 Blockers and Selection of ShK-235 as a Kv1.3 Blocker

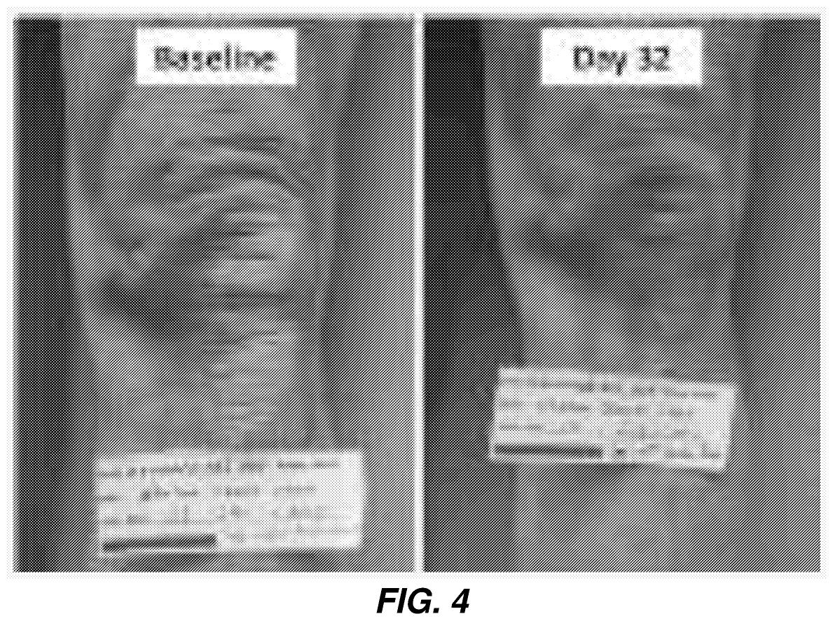

ShK was identified in the venom of the sea anemone Stichodactyla helianthus as a potent blocker of Kv1.3 with an IC50 of 10 pM2-4 (FIG. 3B, SEQ ID NO:10). ShK also blocks other K⁺ channels. The inventors modified ShK by adding a pTyr to its N-terminus via an AEEA linker to generate ShK-186, now known as dalazatide (FIG. 3B, SEQ ID NO:11). This modification maintained the affinity of the peptide to Kv1.3 (IC₅₀=70 pM) and improved the selectivity for Kv1.3 as ShK-186 is 100-fold selective for Kv1.3 versus the closely-related Kv1.1 channel and 700-fold or more selective over all other ion channels. ShK-186 has demonstrated an excellent safety profile after systemic administration in rodents, non-human primates, healthy humans, and patients with an autoimmune disease during Phase 1A and 1B clinical trials^{6,8,16,37}. In addition, ShK-186 did not prevent the clearance of viral or bacterial infections in rats or induce the reactivation of CMV in non-human primates^{8,38}. In addition to its safety profile, ShK-186/dalazatide demonstrated efficacy in rat models of RA and multiple sclerosis^{6,8,33,37,40}. ShK-186 was well tolerated and showed efficacy in a Phase 1B clinical trial in patients with active plaque psoriasis resistant to current medications at doses as low at 30 μg/patient 3 times a week, administered subcutaneously (FIG. 4)¹⁸.

The AEEA linker in ShK-186 prevents its recombinant expression in bacteria. The inventors therefore designed ShK-235 that differs from ShK-186 by only 3 amino acids (FIG. 3B, SEQ ID NO:12)¹, lacks the pTyr-AEEA linker, retains the advantages of ShK-186 in terms of high affinity and selectivity for Kv1.3, and can be produced recombinantly. ShK-235 has the following characteristics that make it an attractive compound for testing the inventors' novel delivery system:

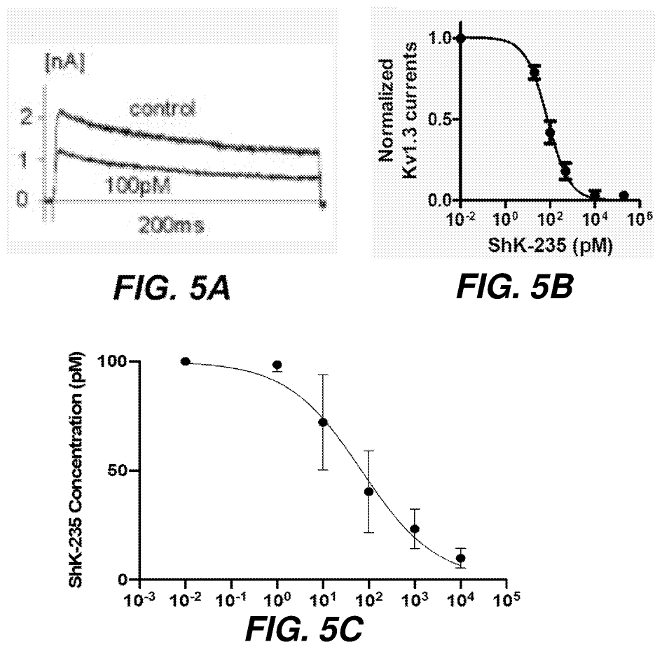

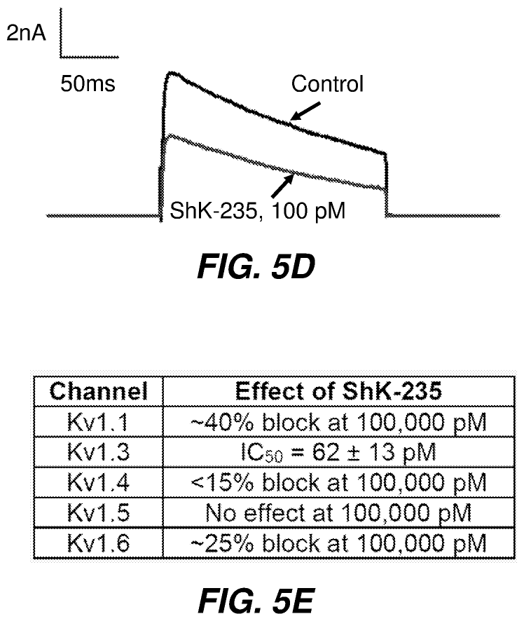

1. Potency on Kv1.3. Like ShK-186, ShK-235 is highly potent in blocking Kv1.3 channel function with an IC₅₀ of 62 pM on Kv1.31 (ShK-186's IC₅₀ is 70 pM)³ (FIGS. 5A-5B, 5E).

2. Improved selectivity for Kv1.3. ShK-235 is more selective than ShK-186 for Kv1.3 over other channels with a 2,200-fold selectivity or more¹ compared to 100-fold for ShK-1863 (FIG. 5E).

3. Preferential targeting of CCR7⁻ T_{EM} lymphocytes. As a selective Kv1.3 blocker, ShK-235 preferentially inhibits the activation of human and rat T_{EM} cells with a similar IC₅₀ and has little to no effect on the activation and function of human and rat naïve and T_{CM} cells¹.

4. Ease of expression in bacteria. Unlike ShK-186 that contains an unnatural adduct³, ShK-235 is composed of only natural amino acids (FIG. 3B) and can thus be expressed recombinantly¹. As a non-glycosylated small peptide, it is easily expressed by bacteria¹.

3. Selection and Generation of Lactobacillus reuteri as a Bioengineered Delivery Vehicle of ShK-235

To generate a L. reuteri strain that is capable of secreting ShK-235, the gene for ShK-235 was codon-optimized for expression in L. reuteri, fused to the usp45 signal peptide sequence at the 5′ end, and the fusion product was cloned into the vector pSIP411, resulting in plasmid pLL01 (FIG. 3A). pSIP411 allows for the controlled expression of genes by the addition of a peptide pheromone that activates a promoter, based on a two-component regulatory system identified in L. sakei (see FIG. 3A), and ShK235 expression is induced upon addition of the SppIP peptide pheromone. The resulting bacterial strain containing the pLL01 was named LrS235 (L. reuteri ShK-235). As a control for subsequent experiments, pSIP411 was used, which contains the gusA gene that encodes a B-glucuronidase (bacteria strain referred to as LrGusA hereafter). Induction of neither ShK-235 nor GusA secretion had any measurable effect on cells growth.

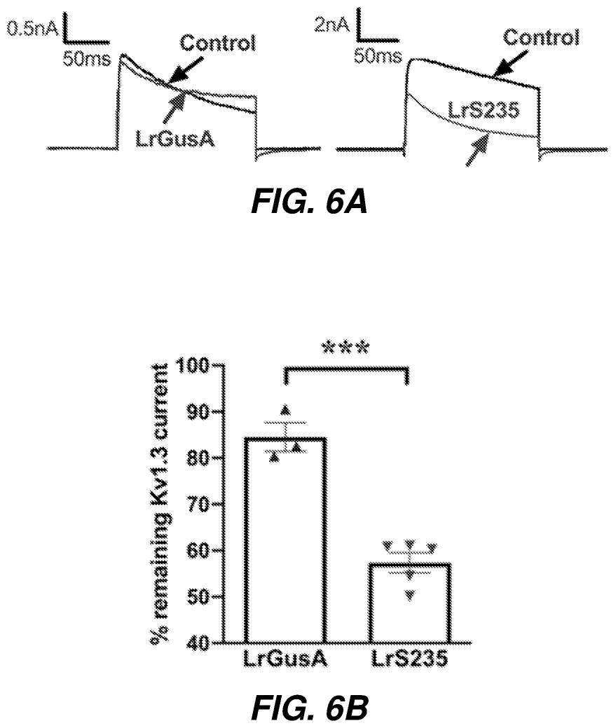

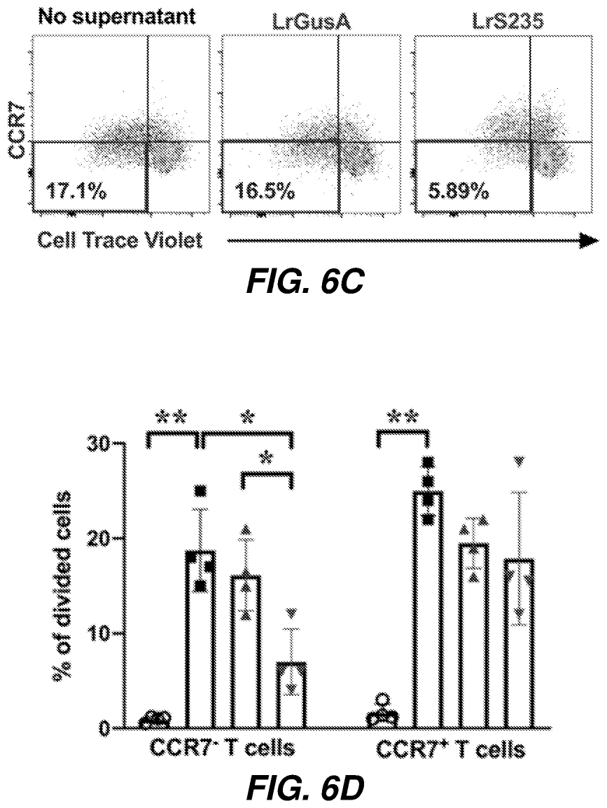

In the absence of antibodies directed to ShK-235, the inventors used functional assays to identify and quantify ShK-235 expression and secretion into culture supernatants of LrS235. In more detail, the presence of active peptide was assayed by using whole-cell patch-clamp to test the ability of LrS235 supernatants to block Kv1.3 channels in mouse L929 fibroblasts stably expressing mKv1.3 channels. LrS235 and LrGusA were grown to mid-exponential phase and induced for expression of ShK-235 or GusA, and supernatants were processed as described in the materials and methods. As an additional control, known concentrations of synthetic ShK-235 were used to plot a dose-response curve of the block of Kv1.3 by ShK-235 (IC₅₀=69±24 pM; FIGS. 5C, 5D). Culture supernatant from LrS235 and LrGusA was tested for Kv1.3 channel block and the dose-response curve used to calculate the concentration of ShK-235 in supernatants. Using single-cell patch-clamp electrophysiology, the inventors found that the culture supernatants of LrS235, but not of LrGusA, blocked Kv1.3 currents (FIG. 6A). The mean concentration of ShK-235 in the supernatants from LrS235 was calculated to be 421.3±75.9 pM (FIGS. 5C-5D, 6A-6B), well above the IC₅₀ of ShK-235 for Kv1.3.

The inventors next tested these supernatants, at a 1/100 dilution in tissue culture media, on the proliferation of primary human T_{EM} cells. The culture supernatants of LrS235, but not of LrGusA, preferentially inhibited the proliferation of human CCR7⁻ T_{EM} cells by 63%, confirming the presence of biologically active and physiologically relevant ShK-235 peptide (FIGS. 6C-6D).

### 4. Oral Delivery of LRS235 Reduces Disease Severity in Collagen-Induced Arthritis Model

Since functional ShK-235 was detected in vitro and in the circulation of rats, efficacy of ShK-235 was next assessed in a model of arthritis in Lewis rats induced by porcine collagen II as described^{36,66}. Starting at onset of clinical signs, four groups of rats received ˜1×10⁹ CFU of either LrGusA or LrS235, both after SppIP induction for 2.5 hr, by daily oral gavage, or P6N vehicle or synthetic ShK-235 by injection in the scruff of the neck every other day.

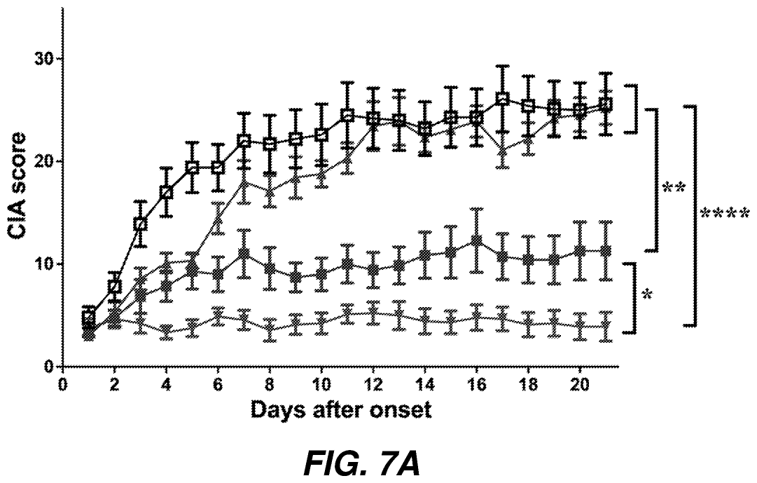

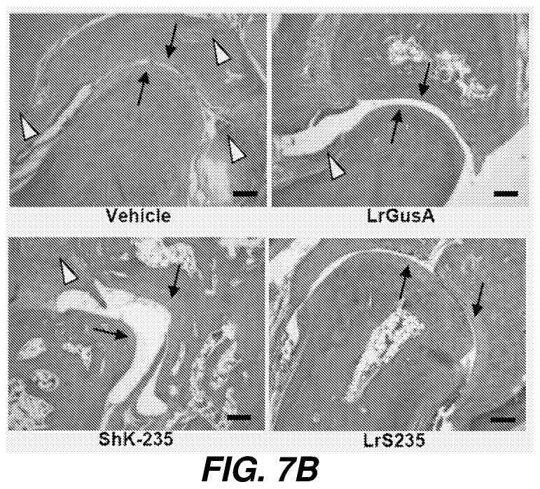

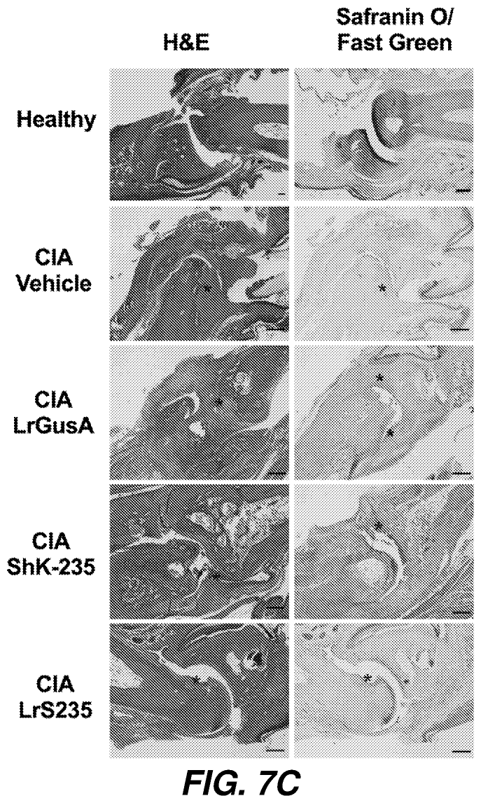

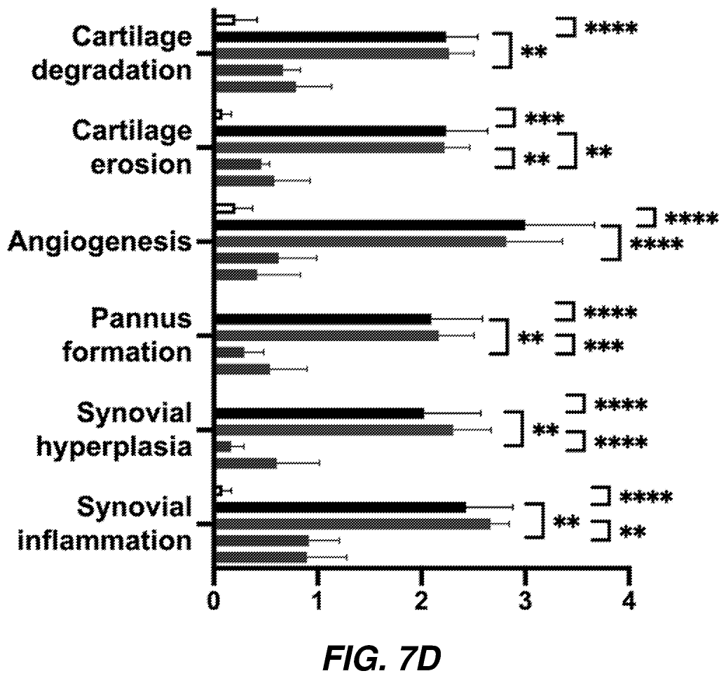

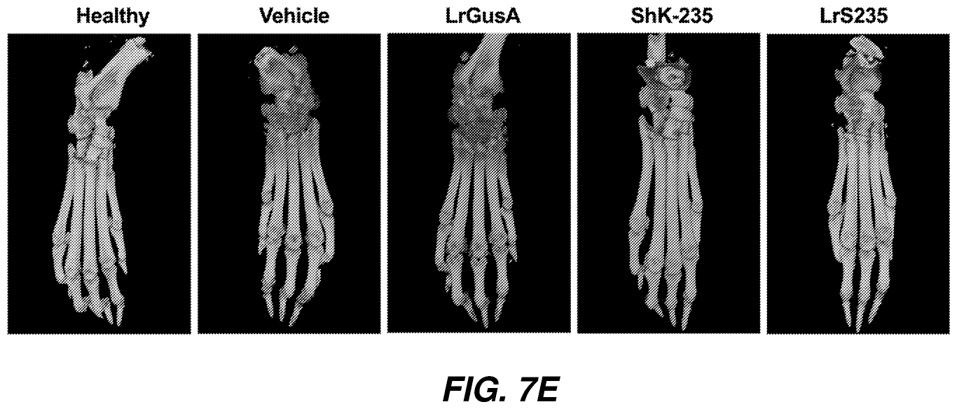

All vehicle-treated animals developed severe arthritis with a mean score of 26±3 (FIG. 7A). The administration of LrGusA did not affect overall disease severity (mean score 25±2). In contrast, the injection of synthetic ShK-235 reduced the mean score by ˜60% to 11±3. The administration of LrS235 was even more effective with a mean score of only 4±1, or an 84% reduction when compared to the vehicle control group. Histology performed on joints collected at the end of the in vivo trials showed severe cartilage degradation and erosion, angiogenesis, pannus formation, and synovial hyperplasia and inflammation in the CIA control rats (FIGS. 7B-7D). Whereas LrGusA had no benefits in any of these parameters, both synthetic ShK-235 and LrS235 significantly reduced all parameters, and no significant differences were seen between healthy controls and ShK-235 or LrS235 treated groups (Table 1).

TABLE 1

| Parameters | Healthy | LrGusA | ShK-235 | LrS235 |
|---|---|---|---|---|
| Synovial | <0.0001**** | 0.9853 | 0.0247* | 0.0097** |
| inflammation | | | | |
| Synovial | 0.0002*** | 0.9731 | 0.0453* | 0.0007*** |
| hyperplasia | | | | |
| Pannus formation | <0.0001**** | 0.9999 | 0.0219* | 0.0011** |
| Angiogenesis | <0.0001**** | 0.9947 | <0.0001**** | <0.0001**** |
| Cartilage erosion | <0.0001**** | >0.9999 | 0.0123* | 0.0013** |
| Cartilage | 0.0002**** | >0.9999 | 0.0406* | 0.0065** |
| degradation | | | | |

P values for 2-way ANOVA analysis, compared with vehicle-treated CIA rats. 2-way ANOVA analysis was performed, followed by Tukey's multiple comparisons test, with individual variances computed for each comparison. *P < 0.05; **P < 0.01, ***P < 0.001, ****P < 0.0001.

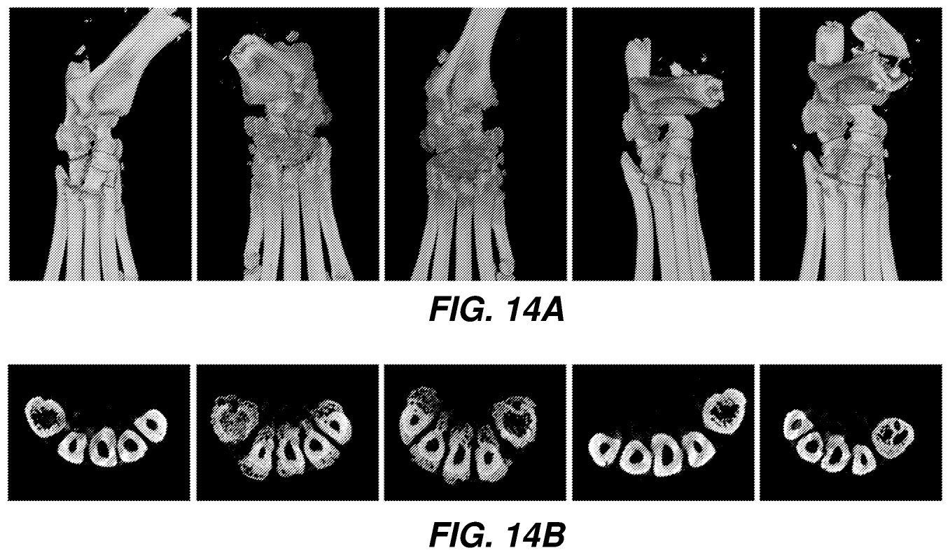

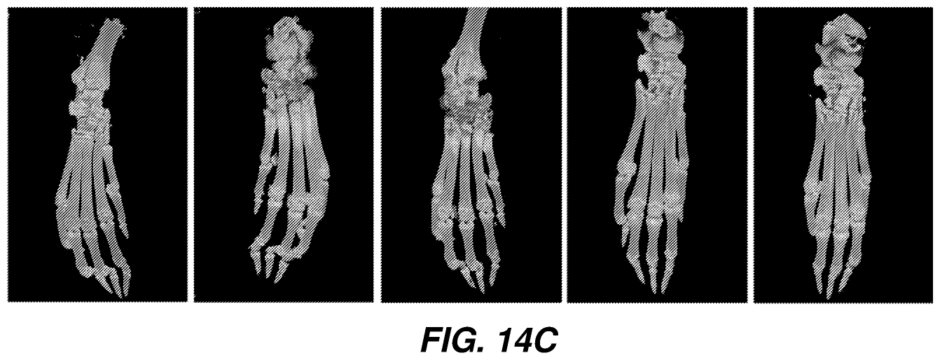

Micro-CT imaging of hind limbs shows severe bone erosions in the CIA rats treated with vehicle or LrGusA, and better-preserved bones in CIA rats treated with synthetic ShK-235 or LrS235 (FIG. 7E). The zoomed, axial, and pseudocoloral images are shown in FIGS. 14A-14C.

### 5. Oral Delivery of LRS235 Yields Functional Levels of SHK-235 in Circulation

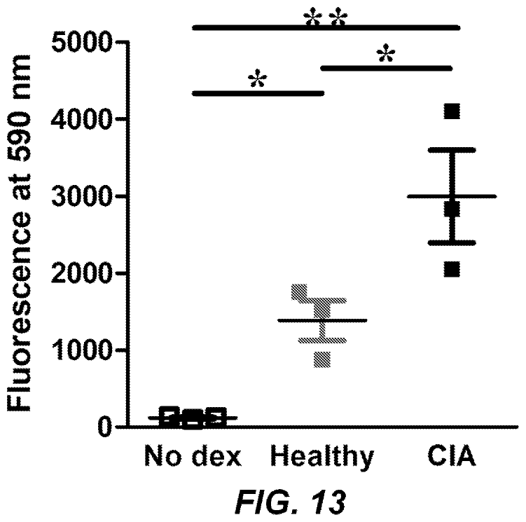

FIG. 6 shows that LrS235 can secrete functional ShK-235 in culture. Before testing whether oral gavage of the probiotic efficiently delivers functional ShK-235 to the circulation of rats, it was first tested whether a compound of a molecular weight similar to that of ShK-235 can cross from the lumen of the GI tract into the circulation of healthy rats and rats with the collagen-induced arthritis (CIA) model of RA. The oral gavage of healthy rats or rats at the onset of CIA with 4 kDa dextran labeled with FITC showed that dextran could reach the circulation of both healthy and arthritic rats within 6 hours, with a higher permeability in the latter (FIG. 13).

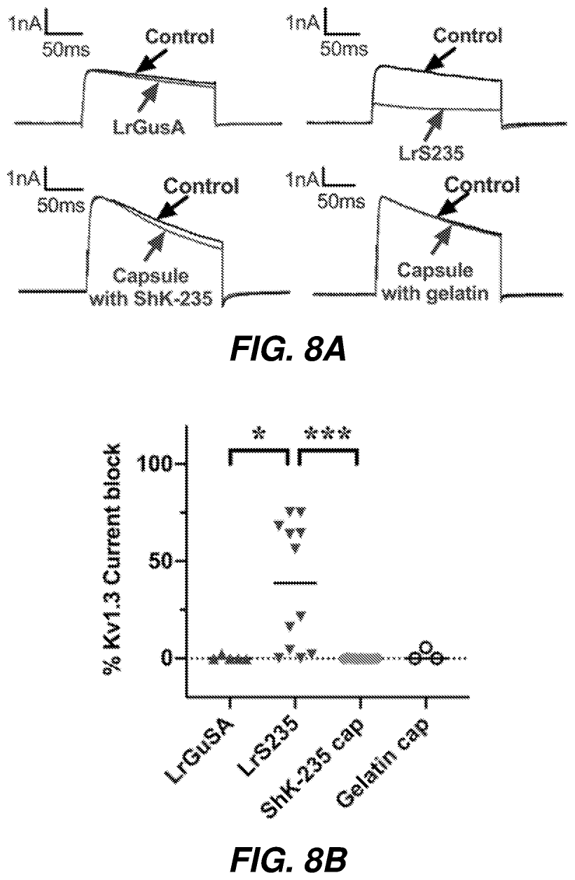

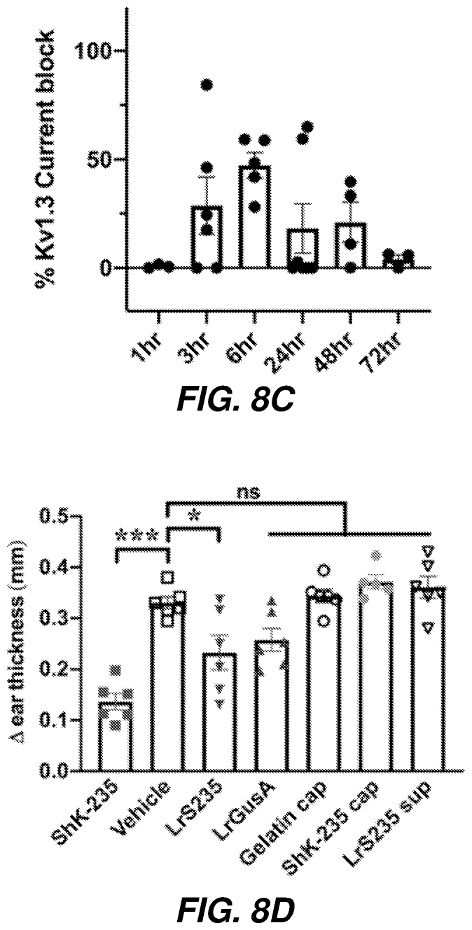

To test whether the intestinal environment interferes with LrS235 secretion of ShK-235, or prevents it from crossing into the circulation, healthy rats were gavaged with a single bolus of 1×10⁹ CFU of either LrGusA or LrS235, both after SppIP induction, and 6 hours later, sera was collected and the ability of serum samples to block Kv1.3 channels tested by whole-cell patch-clamp. Whereas serum from rats gavaged with LrGusA did not affect Kv1.3 currents, serum from rats gavaged with LrS235, diluted 1/100, reduced Kv1.3 currents by 37.2% (FIGS. 8A-8B). Based on the dose-response curve, the calculated concentration of LrS235 in the circulation 6 hours after a single oral gavage is 15.8 nM, significantly higher than the IC₅₀ for Kv1.3 block of 69.31±24.0 pM (FIGS. 5C-5D), and higher than what is detected in the circulation of rats after an injection of 0.1 mg/kg (0.6-0.8 nM). In contrast, oral administration of a Torpac size 9 h gelatin capsule coated with ACRYL-EZE® to target content delivery to the small intestine and filled with 0.3 mg ShK-235 (20 times the dose injected for efficacy in CIA) or unflavored gelatin powder yielded no measurable serum levels of ShK-235 (FIGS. 8A-8B).

Next, the pharmacokinetics of ShK-235 after oral delivery of LrS235 was determined by providing LrS235 by single oral gavage (10\hat{ }9 cfu/ml) to healthy rats and measuring blockage of Kv1.3 channels by whole cell patch clamp. Sera was taken at various timepoints ranging from 3 hours to 72 hours after the single bolus of LrS235. Peak levels of ShK-235 were detected 6 hours after LrS235 delivery, with the range of functional activity from 3 hours to 48 hours post-gavage (FIG. 8C). These results demonstrate bacterial peptides delivered to the gut can enter the serum and remain functional.

Then, it was assessed if LrS235 gavage could reduce a delayed-type hypersensitivity (DTH) reaction in the ear of rats, a local autoinflammation mediated by antigen-specific TEM lymphocytes. Oral gavage of LrS235 and the injection of ShK-235 significantly reduced inflammation by 30% and 58%, respectively, compared with the vehicle control (FIG. 8D). In contrast, treatment with LrGusA, capsules filled with ShK-235 or gelatin, and supernatants from the culture of the LrS235 had no effects on inflammation.

In summary, these data demonstrate the successful secretion of active ShK-235 by LrS235 in sufficient amounts to block Kv1.3 channels, inhibit the proliferation of human T_{EM} cells, and reduce disease severity in a rat model of RA. A probiotic-based delivery of active ShK-235 by L. reuteri presents several advantages. In addition, in some aspects, L. reuteri continuously delivers ShK-235 throughout the GI tract. Furthermore, in some aspects, L. reuteri has an affinity to mucus and will deliver the peptide in close proximity to the intestinal epithelium.

### 6. Therapeutic Benefit of LRS235 in Rheumatoid Arthritis

Kv1.3 blockers, including ShK analogs, reduce disease severity in rat models of RA after systemic administration via subcutaneous injections in the scruff of the neck^{8,36,37,40}. Repeated injections of biologics over years poses risks to patients, including pain and infection, and brings social stigma with handling of needles, leading to a high rate of non-compliance with prescriptions and thus disease worsening. The inventors demonstrate herein a novel oral delivery method using an engineered probiotic to secrete the Kv1.3 blocker ShK-235. Initial data show that L. reuteri produced ShK-235 is found in the circulation (FIG. 8) and the efficacy of LrS235 in treating CIA (FIGS. 7 and 14). The pharmacokinetics and biodistribution of LrS235-delivered ShK-235 and its benefits in preventing and treating two rat models of RA is assessed and the claimed delivery method compared to the traditional subcutaneous delivery and oral peptide delivery via an enteric-coated capsule.

All data is presented as mean±SEM. Statistical analyses are performed using independent, two sample t-tests (GraphPad Prism software). Normality is assessed using qq plots; in case of obvious departure from normality, data transformations (e.g. natural logarithm) are used. P<0.05 is considered statistically significant. Comparisons involving time courses are performed with a two-way ANOVA followed by Tukey's post-test for multiple comparisons. P values <0.05 are considered statistically significant. Outliers are detected by applying Grubbs' method with a=0.05 to each group. Experiments with readouts that can be subject to potential individual bias (e.g. CIA and PIA scoring or histology scoring) are performed by investigators blinded to the test conditions.

Animal models. A rat model is used for experiments testing the therapeutic benefit of LRs235 in rheumatoid arthritis. Rat T-cells mimic human T-cells in terms of Kv1.3 and KCa3.1 channel phenotype and function during differentiation^{2,6,9,74}. In contrast, mouse T-cells express more K⁺ channels than just Kv1.3 and KCa3.1, do not upregulate Kv1.3 during differentiation into T_{EM} cells, and display little sensitivity to Kv1.3 blockers^{2,74}. Further, to best recapitulate human disease, two complementary animal models, pristane-induced arthritis (PIA) and CIA, are used. PIA models many of the characteristics of RA: (a) it is a systemic disease that mainly affects diarthrodial joints; (b) it is chronic-relapsing; (c) it has a strong autoimmune component mediated by MHC class II restricted T-cells; (d) gene loci that control onset, severity, and chronicity of PIA are associated with RA; and (e) CCR7⁻ T_{EM} cells participate in the pathogenesis of RA and of both PIA and CIA. CIA remains one of the most widely used models of RA for testing potential new therapeutics as it also recapitulates many features of RA: (a) it is an autoimmune-mediated synovitis followed by erosion in bone and cartilage; (b) the adaptive immune compartment involves both T and B cells; and (c) plasma levels of anti-cyclic citrullinated peptide IgG are elevated. In addition, the inventors have shown that Kv1.3 blockers are effective in stopping disease progression in both PIA and CIA^{8,36,37,40}.

A power analysis showed that a minimum sample of 12 rats per group would be sufficient to generate statistically significant data with 80% power to detect a 50% reduction in disease severity assuming α=0.05. Since the incidence of CIA in Lewis rats and of PIA in DA rats is ˜80%, 25% more rats than needed per group are immunized. Each assay is done twice to ensure reproducibility of results.

Rats are randomly assigned to groups based on sex and body weight. For assays using rats with CIA or PIA, rats are also randomized by disease severity.

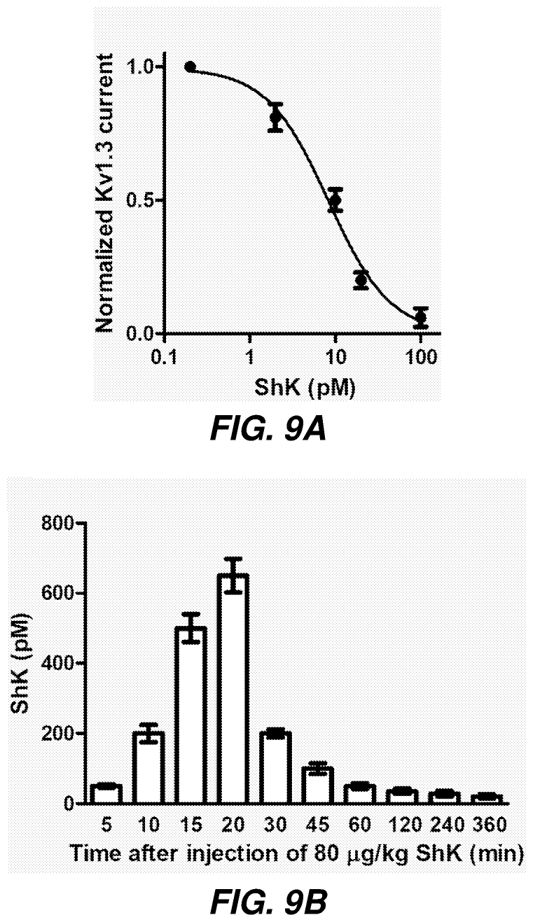

Pharmacokinetics following oral or subcutaneous administration of LrS235. Pharmacokinetics following oral or subcutaneous administration of LrS235 are conducted to determine the efficacy of ShK-235 entry into the circulation after its delivery via LrS235, an oral capsule, or subcutaneous injection, and the duration for which the peptide can be detected in the blood after a single bolus or repeated administration to rats with the CIA model of RA. The circulating levels of ShK-235 are quantified with two complementary and well-established techniques, mass spectrometry and single-cell patch-clamp, as the inventors and others have done with ShK, ShK analogs, and other Kv1.3 channel modulators^{6,16,37,47-49,65}. Tandem liquid chromatography triple quadrupole mass spectrometry (LC-QQQ-MS) is commonly used to rapidly detect and quantify small molecules and peptides for pharmacokinetics. As a complement, single-cell patch-clamp allows for the detection and quantification of functional peptide. This quantitative method (FIG. 9) has a low pM detection limit for the functional peptide. This technique has been validated and used to detect ShK-235 in the circulation of rats administrated the peptide via a capsule or via LrS235 (FIG. 8).

CIA is induced in Lewis rats by two injections at a week interval of an emulsion of porcine collagen II in incomplete Freund's adjuvant at the base of the tail^{36,40,65,66}. Clinical signs of CIA are observed, in some aspects, 3-6 days after the booster injection. For single-dose pharmacokinetics, healthy rats and rats with CIA receive a single oral bolus of 5×10⁹ CFU LrS235 or 2 mg/kg ShK-235 in an TORPAC® 9 h capsule coated with ACRYL-EZE® enteric coating (COLORCON®), or a subcutaneous injection of 100 μg/kg ShK-235 10 days after onset of clinical signs. As controls, rats receive LrS235H19A (5×10⁹ CFU), L. reuteri expressing a mutant of ShK-235 in which the histidine in position 19, crucial for binding of the peptide to Kv1.3, is replaced by an alanine, creating a mutant that cannot block Kv1.375. Blood is collected 30 min, 1 hr, 2 hrs, 4 hrs, 6 hrs, 16 hrs, 24 hrs, 48 hrs, and 72 hrs after administration. For repeated-dose pharmacokinetics, this oral dosing occurs daily for 7 days and blood is collected 1 hr, 6 hr, and 1, 3, 7, and 14 days after the last administration. Plasma is aliquoted and frozen at −80° C. For LC-QQQ-MS, samples are provided in a blinded manner, along with control samples of rat plasma spiked with known concentrations of ShK-235. Samples in which LC-QQQ-MS detects the peptide are used for patch-clamp in which samples are tested for their ability to block Kv1.3 currents using an automated patch-clamp system that allows small test volumes (10-50 μL). Results are compared against a dose-response curve using naïve Lewis rat plasma spiked with known amounts of ShK-235 as in FIG. 9^{6,47-49,65}.

To test the ability of Kv1.3-blocking rat plasma to preferentially inhibit CCR7⁻ T_{EM} cell proliferation, allowing CCR7⁺ naïve and T_{CM} cells to escape inhibition^{4,73}, de-identified buffy coats are used to isolate mononuclear cells. Cells are loaded with CELL TRACE™ Violet and stimulated with anti-CD3 antibodies in the presence of 56° C. heat-inactivated 10% plasma from rats (ShK-235 is stable up to 80° C.). Rat plasma spiked with ShK-235 (10 pM-100 nM) is used as a positive control. After a 72 hr culture, cells are stained with anti-CD3, anti-CD45RA, and anti-CCR7 antibodies for flow cytometry. Gating is on CD3⁺CD45RA⁺CCR7⁺ naïve T-cells, CD3⁺CD45RA⁻CCR7⁺ T_{CM} cells, and CD3⁺CD45RA⁻CCR7⁻ T_{EM} cells and measure Cell Trace Violet dilution as a readout for T-cell division³⁶.

A power analysis showed that a minimum sample of 8 per group would be sufficient to generate statistically significant data with 80% power to detect a difference between two groups using an independent, two-sample t-test, assuming α=0.05.

In a specific aspect, ShK-235 is detected in the circulation of rats treated with LrS235 and injected with ShK-235, but not from rats with any of the other treatment conditions. In a specific aspect, the presence of ShK-235 in the plasma is of longer duration in rats treated with LrS235 than after peptide injection. In a specific aspect, ShK-235 detected in the circulation blocks Kv1.3 channels and inhibits the proliferation of human T_{EM} cells. No long-term accumulation of ShK-235 in the circulation is observed in some aspects because any peptide not bound to Kv1.3 channels is eliminated by kidney filtration, as previously shown with ShK-186 and other ShK analogs^{6,8,36,37}. Finally, detection of ShK-235 in the circulation is observed in some aspects after the capsule-based delivery of ShK-235 but at lower levels and much more transiently than after LrS235-based delivery.

Early and late treatment of CIA and PIA by LrS235. The objective of these experiments is to define the benefits of LrS235 as an oral delivery system for ShK-235 in reducing joint damage and local and systemic inflammation in rat models of RA and compare its efficacy to that of injected ShK-235 and ShK-235 delivered via an enteric-coated capsule. CIA is induced in Lewis rats by two injections at a week interval of an emulsion of porcine collagen II in incomplete Freund's adjuvant at the base of the tail^{36,40,65,66}. PIA is induced in DA rats by the single injection of pristane at the base of the tail. Clinical signs of CIA are observed in some aspects 3-6 days after the booster injection and clinical signs of PIA are observed in some aspects 2-3 weeks after the pristane injection. Clinical signs are scored daily based on the number of joints affected and the severity of the swelling, as in FIG. 7A. In particular, each paw of each rat is examined daily by a person blinded to the treatment group. A score of 1 is given for each red or swollen toe of knuckle joint, a 2 for each mildly swollen ankle or wrist, and a 5 for each severely swollen ankle or wrist. The maximum score is 15 per paw, or 60 per rat.

In each experiment, there are 6 treatment groups of rats:

1. The engineered probiotic. LrS235, the bioengineered probiotic, daily oral dose determined from the pharmacokinetics assays.

2. A capsule-based delivery. Synthetic ShK-235, daily oral dose determined from the pharmacokinetics assays, in a TORPAC® 9 h capsule coated with Acryl-EZE® enteric coating (COLORCON®).

3. Positive Control. Synthetic ShK-235, daily subcutaneous dose determined from the pharmacokinetics assays.

4. Negative Control 1. LrGusA (L. reuteri expressing a secreted beta-glucuronidase A on the same plasmid backbone as Shk-235), same daily oral dose as LrS235.

5. Negative Control 2. LrS235H19A (L. reuteri expressing a mutant of ShK-235 in which the histidine in position 19, crucial for binding of the peptide to Kv1.3, is replaced by an alanine, creating a mutant that cannot block Kv1.375), same daily oral dose as LrS235.

6. Negative Control 3. P6N buffer (the vehicle for ShK-235 [10 mM NaHPO₄, 0.8% NaCl, and 0.05% polysorbate 20, pH 6.0] used for clinical trials with ShK-186/dalazatide^{16,37}), daily subcutaneous dose.

Rats start receiving daily treatments either at onset of clinical signs (early treatment), or 10 days later, at peak of disease severity (late treatment). In all assays, treatment continues for 4 weeks. Rats are monitored daily for body weight and scored for clinical signs of arthritis using a standard scoring method^{36,40,65,66} described in herein (FIG. 7A). A plethysmometer is also used to measure hind paw volumes twice weekly. At the end of the trials, animals are euthanized for collection of blood, hind limbs, inguinal lymph nodes, and small and large intestines, and the following is measured:

1. Circulating markers of inflammation. The blood is used to prepare plasma for the quantification of rheumatoid factor, a marker for RA and its rat models. RA and its models are mediated by Th1 and Th17 T_{EM} cells, and blocking Kv1.3 inhibits the production of Th1 and Th17 cytokines by T_{EM} cells^{6,8,76}. Plasma levels of Th1 (IFNγ and TNFα) and Th17 (IL-17A) cytokines are quantified as an indicator of reduction in systemic inflammation. Blocking Kv1.3 also biases T-cells towards an immunoregulatory phenotype characterized by the increased production of IL-10^{17,77}, and therefore plasma IL-10 levels are also quantified.

2. Joint inflammation and cartilage and bone damage. Hind limbs are fixed with 10% buffered formalin, imaged by μ-CT to assess bone damage, and sectioned for histology and immunohistochemistry to assess inflammatory infiltrates (hematoxylin-eosin), cartilage integrity (safranin O-Fast green), osteoclasts (tartrate-resistant acid phosphatase), and T-cells (CD3, Kv1.3)^{36,40,65,66}. As shown in FIGS. 7B-7C, joints sections stained with hematoxylin & eosin and/or safranin O/fast green from paws from CIA rats receiving the different treatments is depicted. Histology scoring of the joint sections stained in FIG. 7C is shown in FIG. 7D. Together, these results indicate that, in some aspects, LrS235 stops disease progression, reduces bone and joint damage and inflammation in rats with collagen-induced arthritis. μ-CT scans, shown in FIGS. 7E and 14A-14C, validate the therapeutic effects of engineered probiotic LrS235 on bone destruction in rats with CIA.

3. Quantification of T-cell subsets in inguinal lymph nodes. Inguinal lymph nodes are used for the preparation of single cell suspensions to quantify T_{EM} cells (both CD4⁺ and CD8⁺, and both Th1 and Th17) and their activation status by flow cytometry.

4. Intestinal inflammation and epithelial health. Intestinal sections are stained for Villin and E-cadherin to assess enterocytes and Muc2 to evaluate the mucus layer. Additionally, intestinal inflammation is assessed by staining for T lymphocytes, activated T_{EM} cells, and macrophages.

Based on initial results (FIG. 8A), in a specific aspect, LrS235 is a better delivery system than capsules. In a specific aspect, LrS235 and injected ShK-235 reduce the incidence of CIA and PIA and reduce severity in animals that do develop clinical signs of disease, including reduced levels of circulating rheumatoid factor and proinflammatory cytokines, increased levels of IL-10, reduced infiltrates of activated memory T lymphocytes in the synovium and inguinal lymph nodes, reduced bone and cartilage damage, and reduced number of osteoclasts. A synergistic effect between L. reuteri and ShK-235 in LrS235-treated rats is also observed in some aspects. In a specific aspect, LrS235H19A also has beneficial effects due to L. reuteri's anti-inflammatory and anti-osteoclast properties⁴⁴, but to a much lower level due to ShK235H19A being inactive.

Biodistribution of ShK-235 following the oral administration of LrS235. The objective of these experiments is to define the in vivo localization of ShK-235 after the oral or subcutaneous administration of ShK-235. Initial data show efficacy of LrS235 in reducing disease severity of CIA (FIG. 7) after daily oral administration. Cysteine-rich peptides, such as ShK-235, tend to accumulate in cartilage and joints⁷⁸⁻⁸⁰ and Kv1.3 blockers accumulate at sites of inflammation⁷⁸. Because RA and its animal models are systemic diseases, it is important to understand the biodistribution of the peptide using different methods of administration. Therefore, the localization of ShK-235 in both healthy and CIA rats is determined.

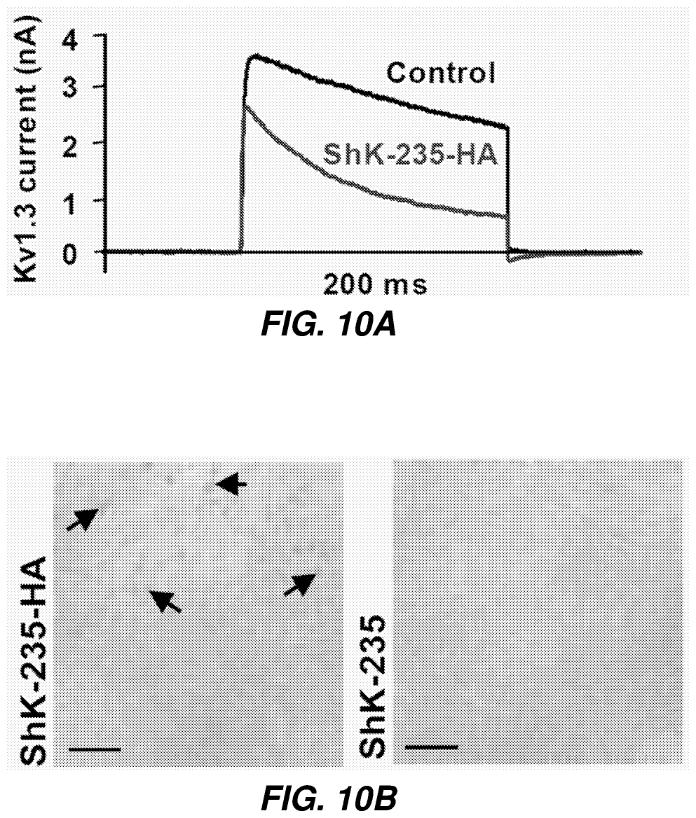

Two complementary approaches to directly detect ShK-235 in joints and tissues and assess its action on T lymphocyte proliferation are used. L. reuteri LJ01 has been engineered to express HA-tagged ShK-235 (LrS235-HA), because the low immunogenicity of the peptide has made it impossible to raise antibodies against ShK-235. In patch-clamp assays, LrS235-HA supernatants blocked peak Kv1.3 currents by ˜25%, demonstrating the production of functionally active HA-tagged ShK-235 (FIG. 10A). ShK-235-HA was injected to healthy rats and their spleens collected. Immunohistochemistry was sensitive enough to detect HA-ShK-235 in spleen sections (FIG. 10B). HA-tagged ShK-235 is used to detect and localize ShK-235 in the joints, intestines, and secondary lymphoid organs of rats with CIA.

Detection of ShK-235-HA by immunohistochemistry and western blots. CIA is induced in rats as described above, and the rats are monitored daily^{36,40,65,66}. Ten days after onset of clinical signs, at peak of disease severity, CIA rats and healthy age- and sex-matched rats are given an oral bolus of LrS235 or LrS235-HA, a subcutaneous injection of ShK-235-HA, or an oral capsule containing ShK-235-HA. Rats are euthanized and cardiac-perfused with saline at the time when the peak of the ShK-235 level is detected in their circulation, or 3 hrs later. The spleen, hind limbs, mesenteric and inguinal lymph nodes are collected, and different sections of the small and large intestines. Half of each tissue is fixed in 10% buffered formalin and the other half is frozen to prepare tissue lysates. Fixed tissue is paraffin-embedded and sectioned for immunohistochemistry, staining for Kv1.3 and HA-tagged ShK-235. The number of positive cells in each location is determined using Nikon image analysis software. Lysates are run by SDS-PAGE in Tris-Tricine peptide gels and transferred to nitrocellulose for western blot against the HA tag. Lysates from LrS235-HA are used as a positive control.

Detection of T lymphocyte inhibition by ShK-235 in vivo. CIA is induced in rats as described above, and rats are treated with either vehicle, ShK-235 (injected or in an enteric-coated capsule), LrS235, LrGusA, or LrS235H19A for four weeks, starting at peak of disease. Rats are monitored daily for clinical signs of disease and receive daily intraperitoneal injections of 5-bromo-2′-deoxyuridine (BrdU) for the last 3 days prior to euthanasia and collection of the spleen, hind limbs, mesenteric lymph nodes, and small and large intestines. Anti-BrdU antibodies are used to detect proliferating cells and T-cell subsets are detected using antibodies to cell surface markers by flow cytometry^{81,82}.

In a specific aspect, in CIA rats, ShK-235-HA localizes to any tissue containing activated Kv1.3^{high} T_{EM} cells during CIA, including inflammatory infiltrates in the colon, spleen, synovial joints, and draining lymph nodes. In a specific aspect, in healthy rats, most ShK-235-HA is excreted, with some remaining in joint cartilage. In a specific aspect, peptide absorption occurs in the small intestine due to the larger surface area and less dense mucus layer. Histological detection of the ShK-235-HA peptide in the submucosa confirms the site of translocation. Detection of T-cell proliferation in the synovium, the intestines, and the secondary lymphoid tissues of rats with CIA is observed in some aspects. Disease in CIA is driven by T_{EM} cells, and therefore, it is observed in some aspects that most of the proliferating T-cells in the different sites of inflammation are T_{EM} cells and that ShK-235, whether injected or produced by LrS235, inhibits the proliferation of these T-cells.

Effect of LrS235 on intestinal permeability during CIA. Data from FIG. 8 shows that ShK-235 produced by LrS235 in the intestinal lumen can cross into the circulation of healthy rats in sufficient amounts to block Kv1.3 channels. In some aspects, intestinal permeability varies with disease severity. This permeability to 4 kDa dextran is assessed before and after CIA onset and correlated with disease severity. CIA is induced in rats as described above. To assess intestinal permeability, rats are given an oral bolus of FITC-conjugated dextran (4 kDa) one day after the second immunization (prior to clinical signs of CIA), on the day of clinical sign onset, or 5, 10, 15 or 20 days after onset. Blood is collected 6 hrs later for plasma preparation and detection of the FITC-dextran with a fluorescent plate reader. As controls, plasma is collected from healthy rats given the FITC-dextran and from healthy and CIA rats given saline instead of the dextran. The saphenous vein is used to draw blood from rats with no or minimal clinical signs of CIA (at least one unaffected hind limb) but, as this procedure is unnecessarily painful for rats with severe CIA, a terminal cardiac puncture is used on these animals.

In a specific aspect, intestinal permeability is higher in rats with CIA than in healthy rats. This permeability varies over time in patients with RA, and therefore, some variability is observed in some aspects between rats and between time-points assessed. In a specific aspect, the intestines remain inflamed during CIA but that the level of inflammation remains stable after onset of clinical signs.

### 7. Assessment and Improvement of the Safety and Efficacy of SHK-235 Delivery

Initial results show that, in some aspects, oral delivery of LrS235 is more effective than injection of ShK-235 for resolving inflammation in the inventors' CIA model. The selectivity of the blocker of Kv1.3 channels and for T_{EM} lymphocytes is an important factor for its benefits in RA and other T-cell-mediated autoimmune diseases. The selectivity of LrS235 products is defined for Kv1.3 over other ion channels and for T_{EM} over naïve and T_{CM} cells. The safety of the repeated delivery of LrS235 is also defined and the ShK235 expression construct integrated into the chromosome to greatly reduce the chance of transfer of this element to other bacteria in the gut.

Large proteins from snake venoms are immunogenic. In contrast, small venom peptides, such as those produced by scorpions, spiders, or sea anemones are rarely immunogenic, which poses specific difficulties in producing anti-venoms to treat stings from medically-relevant scorpions but presents definite advantages for drug design^{87,88}. In addition, ShK is highly homologous in structure and Kv1.3 blocking activity to a domain of MMP-23, thus possibly resembling a self-peptide^{89,90}. ShK-235 differs from ShK by 3 amino acids¹, which could enhance its immunogenicity. Therefore, the immunogenicity of ShK-235 after injection, oral delivery via LrS235 or an enteric-coated capsule is assessed in both healthy rats and rats with CIA as systemic inflammation in some aspects affects the safety and immunogenicity of the peptide.

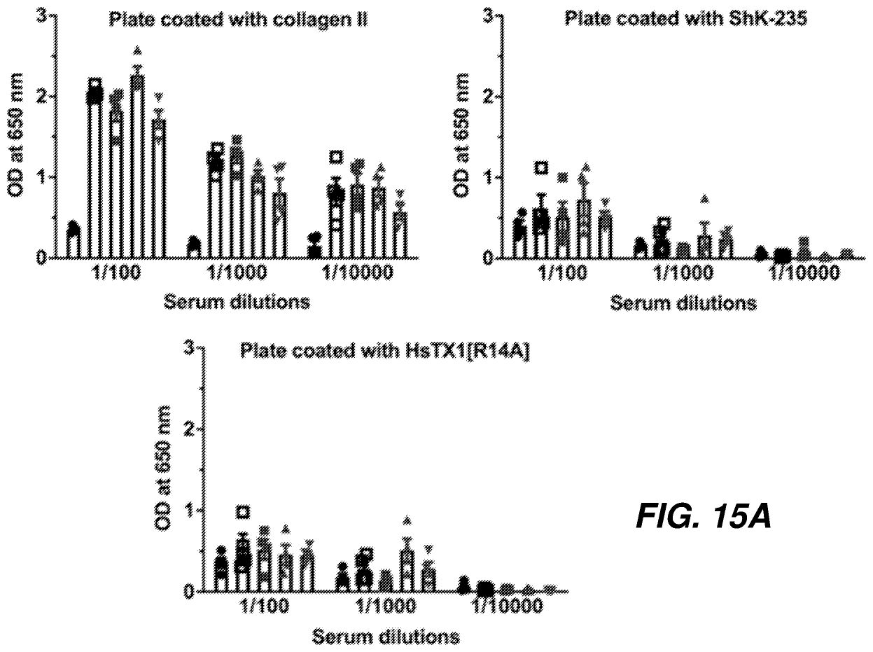

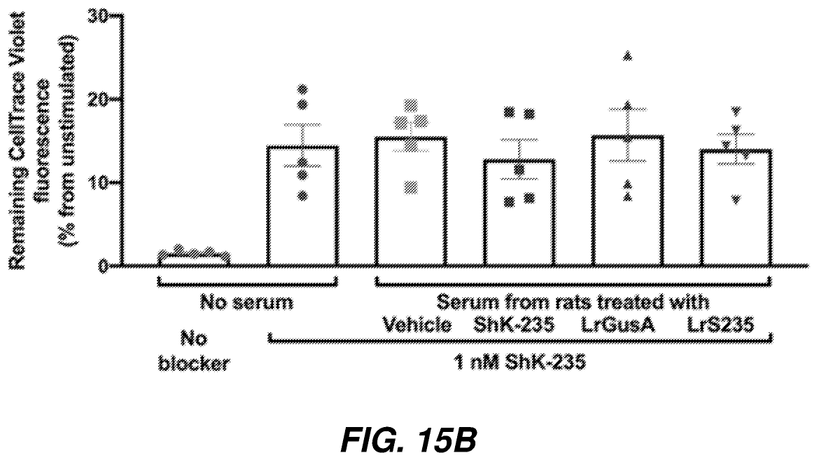

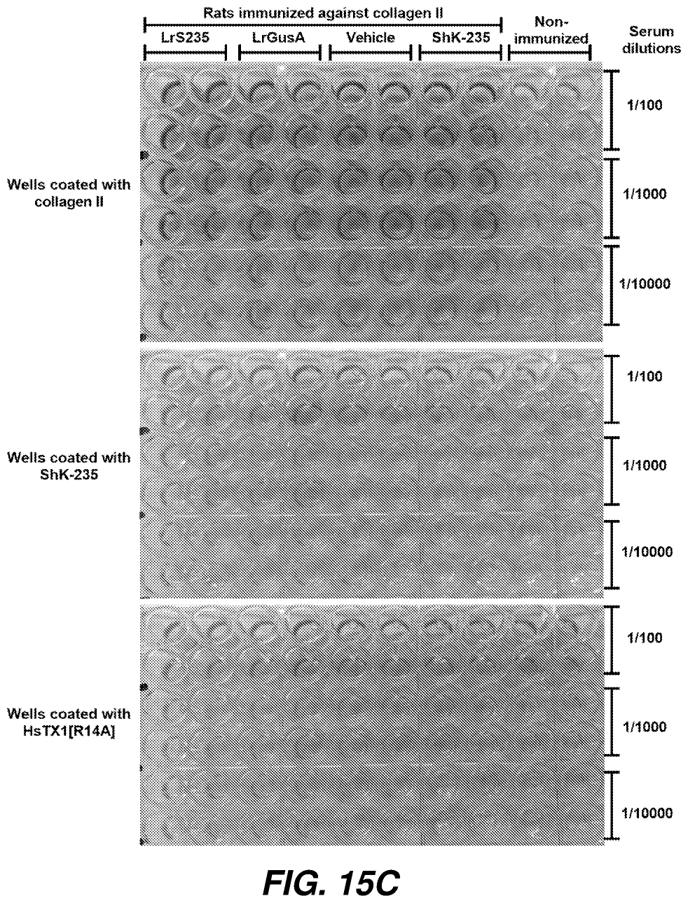

To test if ShK-235 produced by LrS235 is immunogenic, the sera of rats were assessed at the end of the 21-day CIA trial shown in FIG. 8 for anti-ShK-235 IgG by ELISA assay. Plates coated with collagen II and HsTX1[R14A] were used as controls. HsTX1[R14A] is another peptide blocker of Kv1.3 with no sequence or structural homology to ShK-235. High titers of antibodies against collagen II were detected in rats with CIA, but only low titers of anti-ShK-235 antibodies (FIGS. 15A and 15C). Similar low reactivity was observed against HsTX1[R14A], suggesting that the signal detected against ShK-235 was non-specific. A neutralization assay was also performed with the same CIA rat serum samples, and no neutralization of ShK-235 was detected, showing the absence of ShK-235 neutralizing antibodies (FIG. 15B).

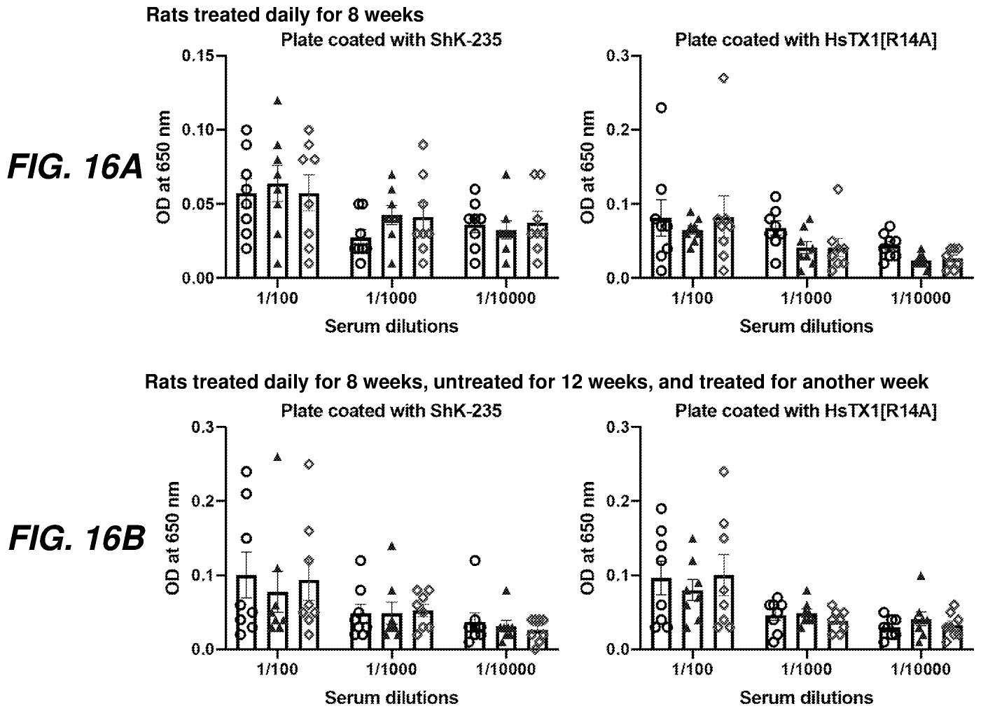

To further investigate if long-term exposure of rats to LrS235 would elicit antibodies against ShK-235, healthy rats were treated daily with LrS235 or LrGusA for 8 weeks, treatment was stopped for 12 weeks, followed by additional daily gavages of either LrS235 or LrGusA for 1 week. ELISA was performed on blood samples collected either at the 8 week or the 21 week timepoints and showed no IgG with specificity for ShK-235 was produced (FIGS. 16A-16B).

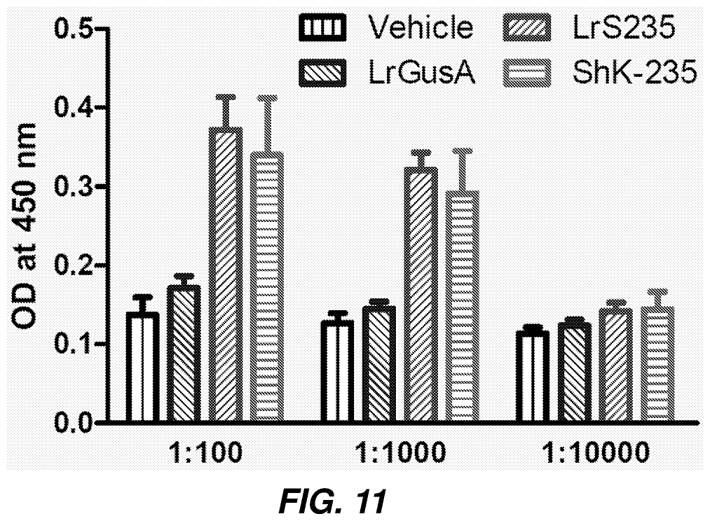

Serum samples were also collected from rats at the end of the CIA trial shown in FIG. 11, and anti-ShK-235 IgGs were assessed, and very low titers of anti-ShK-235 antibodies were detected in rats treated with LrS235 or synthetic ShK-235.

These results suggest that, in some aspects, the B lymphocytes producing these antibodies belong to the CD27⁻IgD⁻ memory subset that relies on KCa3.1 rather than Kv1.3 channels for their function. The frequency of CD27⁻IgD⁻ B cells is increased in patients with both early and established RA and, in some aspects, is likely the subset of B cells producing the IgGs detected in the rats with CIA.

Many drugs, and especially biologics, are immunogenic and the resulting neutralizing antibodies can eventually render the medications ineffective. ShK is highly homologous to a domain of MMP-23 and thus resembles a self-peptide, and in some aspects, is recognized as such by the immune system. Thus, in some aspects, ShK and its analogs induce little to no immunogenicity in rats or humans and no immunogenicity of ShK-235 delivered via LrS235 was found in either healthy or CIA rats, regardless of short or long-term treatment.

Comparison of the selectivity of LrS235-produced ShK-235 with that of synthetic ShK-235. The goal of these experiments is to determine whether LrS235 produces compounds along with ShK-235 that could either affect ShK-235's selectivity or directly affect other ion channels or immune cells. Supernatants are produced from LrS235 and LrS235H19A and single-cell patch-clamp used to assess their effects on a panel of K⁺, Ca²⁺, Na⁺, and Cl⁻ channels in mammalian expression systems^{1,6,7,80,91}. Their effects on the production proliferation of human CCR7⁻ T_{EM} cells and CCR7⁺ naïve/T_{CM} cells activated at rest and after a pre-activation is also tested, as in FIG. 2^{8,9}. The production of cytokines by T_{EM} cells known to be inhibited by Kv1.3 blockers (TNFα, IFNγ, IL-2, IL-17A) and the surface expression of CD25, independent of Kv1.3 function^{8,34}, is also assessed. Ovalbumin-specific rat T_{EM} cells and rat macrophages are used to test the effects of Kv1.3 blockers on antigen processing and presentation and on T-cells proliferation⁹².

Based on initial results, in a specific aspect, LrS235, but not LrS235H19A, supernatants function like synthetic ShK-235 and selectively block Kv1.3 channels and preferentially inhibit the proliferation and cytokine secretion of T_{EM} cells whereas naïve and T_{CM} cells escape, particularly after a pre-activation. In a specific aspect, there are no effects on CD25 expression of antigen processing and presentation by antigen-presenting cells.

Interactions between LrS235 and the gut microbiome. Changes to the intestinal microbiome affect RA pathogenesis in humans and animal models⁸³. For example, gut colonization by Candida albicans aggravates inflammation in CIA⁹⁶ and the oral administration of Porphyromonas gingivalis induces Th17 responses in mesenteric lymph nodes and Peyer's patches and enhances the severity of CIA⁸⁴. Further, early onset RA correlates with increased abundance of Prevotella copri⁸⁵. In mouse models of RA, this dysbiosis contributes to arthritis by increasing the number of Th17 cells systemically and within the intestine⁸⁶. Whether the benefits of LrS235 in CIA could be the result, at least in part and in some aspects, of direct or indirect effects on the gut microbiome are tested.

CIA is induced as described above and the rats monitored daily. Healthy and CIA rats are treated with MRS media alone, or LrS235 or LrS235H19A by oral gavage. Stool is collected from CIA rats 0, 7, 14, and 21 days post-onset of clinical signs of CIA and at similar intervals for healthy rats. Total genomic DNA is extracted from the stool samples and used to measure microbial diversity by 16S ribosomal RNA gene sequencing. The relative abundance of different bacterial families are also compared between the treatment groups, focusing on differences between LrS235H19A and LrS235 over time in both healthy and CIA rats. On day 21, the animals are euthanized and their cecal contents, fecal pellets, and mesenteric lymph nodes collected to detect bacteria that left the intestines.

Differences in the microbiome of rats treated with LrS235 or LrS235H19A would raise the question of whether they are directly due to ShK-235 secretion in the intestines or indirect via modulation of intestinal T_{EM} cell function. Minibioreactor arrays (MBRAs) have been developed for the propagation and functional characterization of the intestinal microbiome^{97,98}. MBRAs are continuous flow bioreactors, and both rat fecal pellets and human feces are used to assess the direct impact of ShK-235 on microbiome composition. Because bacteria also express potassium channels, it is possible that ShK-235 impacts the function of the microbiome. To test the impact of ShK-235 on the microbiome, microbiome communities are derived from feces from healthy rats or healthy human donors. After one week of stabilization, MBRA communities are treated with 100 nM of synthetic ShK-235, LrS235, or LrS235H19A. The composition of the microbial communities are assessed by 16S rRNA sequencing to identify any significant impact on microbial diversity.

In a specific aspect, LrS235 and LrS235H19A minimally affect the microbiome, particularly in the first week following start of treatment, in healthy animals. In a specific aspect, rats with CIA have an altered microbiome, similar to reported in patients with RA and mice with CIA, and LrS235 normalizes this microbiome by reducing intestinal inflammation.

Define the safety and immunogenicity of ShK-235. The goal of these experiments is to test whether repeated administration of ShK-235, particularly that produced by L. reuteri, is safe and whether it induces the generation of neutralizing antibodies. Healthy rats and rats with CIA receive either vehicle, ShK-235 (injected or in an enteric-coated capsule), LrS235, or LrS235H19A 5 days a week for eight weeks. Rats are monitored by an investigator blind to the treatment groups for weight and general appearance (hair loss, fight wounds, hunched posture, edema, tumors, abscesses, head tilt, paralysis, diarrhea, and porphyrin secretion) throughout the treatment period. Half of the rats in each group are randomly selected for euthanasia at the end of the 8 weeks of injection for blood and tissue collection. Blood samples are used for complete blood counts and blood chemistry and tissues are sectioned for hematoxylin/eosin stain and scoring. The remaining rats are kept for another 4 weeks before receiving 5 more boluses of the different treatments over a week before a terminal cardiac puncture⁹³. An ELISA is used to test serum samples from both collection time points for the presence of anti-ShK-235 antibodies as described⁸. Plates are coated with synthetic ShK-235 and mouse anti-rat IgM or IgG used to detect binding antibodies. In a specific aspect, antibodies are detected, and the inventors assess their neutralizing abilities by pre-incubating ShK-235 with the serum before proliferation assays on human CCR7⁻ T_{EM} cells, as described in FIG. 6B.

As with other ShK analogs, in a specific aspect, no changes are observed in gross pathology, blood counts or blood chemistry, and only low titers of anti-ShK-235 antibodies are observed. Based on the results obtained with the closely-related ShK-186 in rats, non-human primates, and humans, neutralizing antibodies are not observed in a specific aspect.

Stable integration of optimized LrS235 constructs into the chromosome. Chromosomal integration is desired as it greatly reduces the chances that the ShK-235 expression construct is transferred to other members of the gut microbiota, thus improving the biosafety of the delivery method. L. reuteri LJ01 was chosen as a platform strain because there are several genetic tools available for placing gene constructs stably into the chromosome. These include CRISPR, recombineering, double crossover allelic exchange, and allelic exchange by counterselection using vancomycin resistance/sensitivity^{70,94,95}. Currently the most robust method is the counterselection method and this is what is used for the proposed experiments. Six sites around the chromosome have been chosen for insertion of constructs to identify the best site for ShK-234 expression. The ability of the constructs to function is assessed by expressing ShK-235-HA from the constructs and measuring expression by western blot. Chromosomal sites that provide the best expression are tested using patch-clamp of culture supernatants to block Kv1.3 channels and to inhibit the proliferation of human T_{EM} cells as described above.

Chromosome position specific expression differences are well known in bacteria, likely due to differences in topological domains that reside in the extreme compaction of the DNA. However, the ability to predict these topological islands and how they affect gene expression is extremely limited, which is why such an empirical approach is proposed.

### Example 2

### Immune Cell KCA1.1 Potassium Channels as Targets of Recombinant Microorganism-Derived Therapeutic for Treatment of Immune System-Mediated Disorders

### 1. Choice of KCa1.1 Channels as Therapeutic Target on FLS

Ion channels represent the third largest family of signal transduction proteins, after protein kinases and G protein-coupled receptors²³ and are actively being pursued as therapeutic targets. A number of ion channels have been identified in primary human FLS, including several Ca²⁺-permeable transient receptor potential (TRP) channels, such as TRPA1, TRPM3, TRPV1, TRPV2, and TRPV4²⁴⁻²⁶. However, these TRP channels are expressed in many cell types, presenting the risk of off-target side effects by therapeutics that modulate TRP channel activity.

The inventors have identified KCa1.1 as the major K⁺ channel expressed by human and rat FLS and found that its expression is enhanced during RA. Blocking KCa1.1 with pharmacological agents or reducing KCa1.1 expression levels with siRNA inhibits the invasiveness, proliferation, and secretion of cytokines, chemokines, proteases, and growth factors by RA-FLS and FLS from rats with the pristane-induced arthritis (PIA) model of RA^{7,8,11}. In contrast, enhancing the function of KCa1.1 with openers induces an increase in FLS invasiveness⁸. Furthermore, the systemic administration of KCa1.1 blockers after onset of clinical signs reduces disease severity in animal models of RA^{8,11,27}. These findings demonstrate that KCa1.1 is a crucial regulator of RA-FLS pathogenic functions during RA.

Like TRP channels, KCa1.1 is also present in many tissues. However, unlike TRP channels, KCa1.1 is an attractive therapeutic target due to the diversity among KCa1.1 regulatory subunits and the expression of different splice variants of the pore-forming KCa1.1 α subunit among different tissues, facilitating the development of tissue-selective pharmacology against KCa1.1 variants²⁸⁻³⁰. Indeed, some modulators of KCa1.1, such as iberiotoxin (IbTX) only affect some splice variants and some subunits of KCa1.1^{7,31-39}.

### 2. Choice of Iberiotoxin (IbTX) as a KCa1.1 Channel Blocker

A functional KCa1.1 channel is composed of a tetramer of α subunits that forms the pore that allows K⁺ ions to cross the plasma membrane. Although a KCa1.1 α tetramer is sufficient to form a functional channel, regulatory β subunits can participate in KCa1.1 channel composition. Four β subunits of KCa1.1 have been described (named β1-4) and the inventors have extensively assessed their expression by human FLS⁴⁰. Whereas the KCa1.1 channel of minimally-invasive FLS, such as OA-FLS, is formed of α and β1 subunits, the KCa1.1 channel of highly-invasive RA-FLS is formed of α and β3b subunits. Haidar et al. showed a similar switch from αβ1 to αβ3 in rat FLS after activation with pro-inflammatory cytokines⁹.

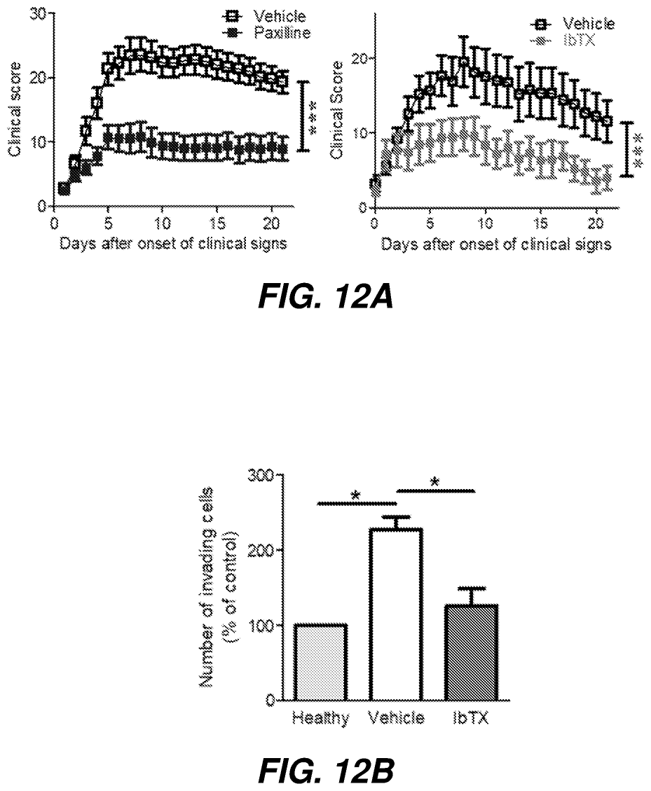

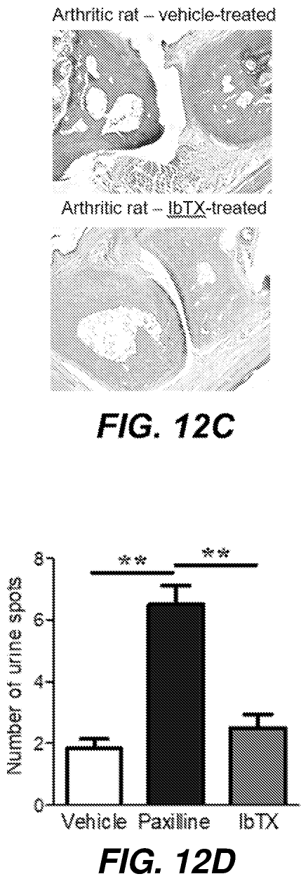

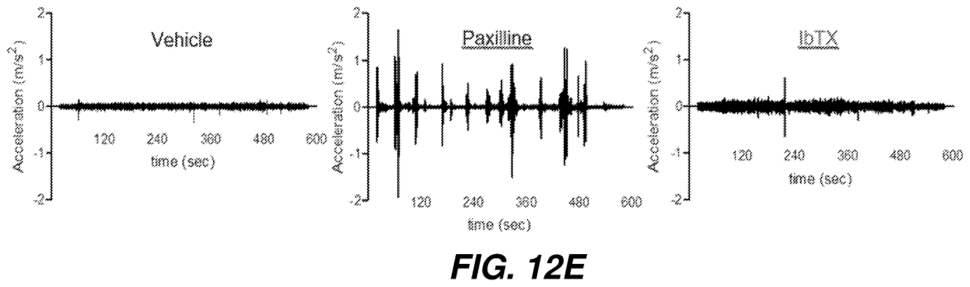

The inventors have tested the efficacy and safety of two KCa1.1 blockers, the small molecule paxilline that blocks KCa1.1 regardless of β subunit and the peptide IbTX that blocks only some αβ combinations of KCa1.1. Whereas both paxilline and IbTX are effective in reducing disease severity in a rat model of RA (FIGS. 12A-12C)^{8,11}, paxilline induces incontinence and tremors by blocking KCa1.1 αβ1 in the bladder smooth muscle cells and KCa1.1 αβ4 in neurons (FIGS. 12A-12C)¹¹. No such side effects are observed after IbTX injection (FIGS. 12D, 12E)¹¹.

3. GI Barrier Permeability to 4 KDA Molecules in Both Healthy Rats and Rats with CIA

To test whether IbTX produced in the gastrointestinal tract can even cross into the circulation, the inventors used 4 kDa dextran (similar molecular weight as IbTX) labeled with FITC and gavaged healthy rats and rats with CIA. The FITC-dextran readily crossed into the circulation of the heathy rat and to an even higher degree, of the rat with CIA, showing that a 4 kDa molecule can cross from the intestinal lumen into the circulation (FIG. 13). Furthermore, Lactobacillus secretes extracellular vesicles that can transverse epithelial cells and transport peptides into the circulation¹⁵.

4. Selection and Generation of Lactobacillus reuteri as a Bioengineered Delivery Vehicle of IbTX

IbTX is a 37 amino acid peptide and is cloned into the vector pSIP411, which allows for inducible expression and secretion of heterologous proteins by L. reuteri (LrIbTX). As a control, L. reuteri is also engineered to produce IbTX[R34D], a mutant IbTX that does not block KCa1.1 channels63. The inventors have shown that L. reuteri can produce and secrete proteins (IL-22; data not shown) and small peptides (FIG. 8) in their active forms. Importantly, both IL-22 and ShK-235 require accurate disulfide bond formation to occur for activity, therefore L. reuteri not only is able to synthesize and secrete these heterologous proteins but also has the proper redox environment for protein folding. Once the strains are created, the strains are grown in buffered MRS medium until mid-log phase and the secreted peptide is allowed to accumulate for 2 hrs in the supernatant.

Whole-cell configuration of the patch-clamp electrophysiology method is used to test the ability of the supernatants of LrIbTX and LrIbTX[R34D] to block KCa1.1 current in HEK 293 cells stably expressing the channel⁷. As the IC⁵⁰ of IbTX for KCa1.1 is known (2 nM)³², the concentration of IbTX in the bacterial supernatants is also calculated. In a specific aspect, LrIbTX produces and secretes sufficient amounts of functional IbTX in the culture supernatant for detection by patch-clamp.

Two prominent characteristics of RA-FLS are their high invasiveness and proliferation, both sensitive to KCa1.1 block. To determine if LrIbTX secretes sufficient amounts of functional IbTX to affect RA-FLS, the inventors will test the ability of the supernatants from LrIbTX to inhibit RA-FLS proliferation and invasiveness.

### 5. Ability of LrIbTX to Secrete Sufficient Amounts of IbTX to Inhibit FLS In Vitro

FLS previously collected from patients with RA undergoing therapeutic joint surgery and banked is de-identified and used for proliferation and invasion assays.

For proliferation assays, RA-FLS are plated into 96 well plates and incubated for 48 hrs in the presence of media supplemented with either LrIbTX or LrIbTX[R34D] supernatants, known concentrations of synthetic IbTX, or the vehicle for IbTX. [3H] thymidine is added during the last 16-18 hrs of culture and the amount of [3H] thymidine incorporated into the DNA collected with a scintillation counter as a measure of cell division.

For invasion assays, RA-FLS are seeded in the upper compartment of a Matrigel-coated transwell and induced to cross into the lower compartment by the addition of FBS. The upper compartment is supplemented with either LrIbTX or LrIbTX[R34D] supernatants, synthetic IbTX, or the vehicle. The number of cells crossing into the lower compartment are counted as a measure of invasiveness.

In one aspect, synthetic IbTX and supernatants from LrIbTX, but not vehicle or supernatants from LrIbTX[R34D], inhibit RA-FLS proliferation and invasiveness.

### 6. Ability of LrIbTX to Secrete Sufficient Amounts of IbTX for Detection in the Circulation of Rats.

Dextran particles of the same molecular weight as IbTX can readily cross from the lumen of the intestines into the circulation of both healthy and CIA rats within 6 hours of oral gavage (FIG. 13). The ability of IbTX produced by LrIbTX to cross into the circulation of healthy rats and of rats with CIA is assessed and compared to enteric-coated capsules.

CIA is induced in both male and female Lewis rats by their immunization twice at a week interval with porcine collagen II in emulsion with incomplete Freund's adjuvant. Rats are monitored for clinical signs of arthritis and receive an oral bolus of [1×10⁷-1×10⁹ CFU] LrIbTX or LrIbTX[R34D], or an enteric-coated capsule containing either 0.1 mg/kg body weight synthetic IbTX, IbTX[R34D] or glycerin on the day after the onset of clinical signs. Age- and sex-matched healthy rats are used for comparison. Blood is collected via the saphenous vein or via terminal cardiac puncture 1 hr, 6 hrs, and 24 hrs after gavage. Whole-cell patch-clamp is used to detect and quantify levels of functional IbTX in the serum of the rats.

In one aspect, IbTX is detected in the circulation of rats gavaged with LrIbTX but not LrIbTX[R34D]. In one aspect, IbTX delivered via capsule at one of the early time points is also transiently detected.

### 7. Efficacy of LrIbTX to Reduce Disease Severity in the Rat CIA Model of RA

Injected IbTX showed efficacy in CIA⁸, and the same model of RA is used to test the efficacy of the oral delivery of LrIbTX in reducing disease severity, bone and joint damage, and inflammation.

CIA is induced in male and female Lewis rats as described above and the rats monitored for clinical signs of arthritis. Treatment begins at onset of clinical signs, at which time rats are randomly assigned to the different treatment groups: oral LrIbTX, oral LrIbTX[R34D], injected synthetic IbTX, and injected vehicle. Treatment continues daily for 3 weeks. Rats are scored daily for CIA severity with a score of 2 given for each lightly swollen ankle or wrist, 5 for each severely swollen ankle and wrist, and 1 for each red and swollen small joint for a total of 15 per paw. At the end of the 3 weeks of treatment and scoring, rats are euthanized for collection of blood and hind limbs. Blood is used to prepare serum to detect circulating levels of IbTX. For each rat, the left hind limb is used for micro-CT to detect bone damage followed by sectioning for histology to detect cartilage damage (safranin O/Fast Green stain) and synovium inflammation (hematoxylin and eosin stain). The right hind limb is used for the isolation of FLS to assess their proliferation and invasiveness ex vivo, using the methods described above, without further treatment.

In one aspect, the vehicle-treated rats have the most severe arthritis. Rats treated with synthetic IbTX have a milder disease, as previously shown¹¹. In one aspect in which the anti-inflammatory properties of L. reuteri benefit rats with CIA, a decrease in disease severity in rats treated with LrIbTX and LrIbTX[R34A] is observed, the rats with LrIbTX having the mildest disease from all the groups as a result from a combination of benefits from L. reuteri and IbTX. In one aspect, the results of joint and bone damage, as well as FLS ex vivo proliferation and invasiveness, match the CIA scoring results in terms of severity.

### Example 3

### Exemplary Materials & Methods

Construct design for the Inducible Expression of ShK-235. Bacteria expressionable ShK-235 peptide was designed which differs from ShK by a Q16K substitution, an I21M substitution, and the addition of an Ala to the C-terminus. Codon optimized ShK-235 with signal peptide Usp45 was synthesized and ligated into NcoI-EcoRI digested pSIP411 generated pLL01. The ShK-235 secretion plasmid pLL01 was enlarged in E. coli 1000 and was then electrotransformed into L. reuteri 647 competent cell resulting in ShK expression strain LrS235. All LrS235 or LrGusA used in this paper were induced with induction peptide unless otherwise stated.

Synthesis of ShK-235 and HsTX1[R14A]. Both Kv1.3-blocking peptides were previously described (16, 44). ShK-235 and HsTX1[R14A] were synthesized using an Fmoc-tBu solid-phase synthesis strategy. Each coupling was mediated with diisopropyl carbodimide in the presence of HOBT. All deprotections were accomplished with 20% piperidine in dimethyl formamide. Following synthesis of the linear chain, each peptide was cleaved and deprotected using trifluoroacetic acid (90%) with carbocation scavengers (triisopropyl silane, H2O, DODT and thioanisole, (2% of each v/v)) for 3 hrs at ambient temperature. The peptides were each precipitated into methyl t-butyl ether. The linear peptides were purified by RP-HPLC and subsequently oxidatively folded in the presence of glutathione in ammonium acetate buffered aqueous solution. The cyclized products were isolated by RP-HPLC and fractions with a purity >95% by analytical HPLC were subsequently pooled and lyophilized. Each peptide was found to have expected theoretic mass for the formation of 3 and 4 disulfide bonds, respectively, for ShK-235 and HsTX1[R14A]. Synthetic ShK-235 and HsTX1[R14A] were dissolved in P6N buffer (10 mM sodium phosphate, 0.8% NaCl, 0.05% polysorbate 20, pH 6.0) to prepare stocks at 1 mg/ml (14, 24).

Animals. Male and female Lewis rats (7-8 weeks old; Envigo, Indianapolis, IN, USA) were group-housed and provided food and water ad libitum.

Patch-clamp electrophysiology. To determine the concentration of ShK-235 in the LrS235 supernatants and rat sera, a dose-response of ShK-235 block of Kv1.3 was determined by adding known concentrations of the peptide to naïve rat serum and then testing by whole-cell patch-clamp of mouse L929 fibroblasts stably expressing mKv1.3 channels (45) using a PORT-A-PATCH® automated patch-clamp system (Nanion, Livingston, NJ), as described (16, 46). Culture supernatants and serum samples were then assessed with the same technique, and the dose-response curve was used to determine peptide concentration.

Human T lymphocyte proliferation assays. Buffy coats were purchased from the Gulf Coast Regional Blood Center (Houston, TX), and mononuclear cells were enriched using HISTOPAQUE®-1077. The cells were loaded with 5 μM CELLTRACE™ Violet (Invitrogen, Waltham, MA) according to the manufacturer's instructions (47) and incubated for 30 min with sterile-filtered supernatants from LrS235 or LrGusA, buffered to pH 7.4 and diluted 1/10 in tissue culture media, before the addition of anti-CD3 antibodies (clone OKT3, 1 ng/ml, 037-85, Thermo Fisher, San Francisco, CA). Seven days later, cells were stained with anti-CD3 antibodies conjugated to phycoerythrin (BioLegend 300308, lot B209105) and anti-CCR7 antibodies conjugated to FITC (R&D Systems, FAB197F, lot LEU1615081) and the dilution of CELLTRACE™ Violet in CD3+CCR7− T_{EM} cells and CD3+CCR7+ naive/TCM cells was measured by flow cytometry on a BD FACSCanto™ II as quantification of cell proliferation. Data were analyzed with FlowJo.

Encapsulation of ShK-235. Torpac size 9 h gelatin capsules (Fairfield, NJ) were filled with either 0.3 mg synthetic ShK-235 or unflavored gelatin powder (Kraft Heinz, Chicago, IL) using the Torpac dosing kit. Each capsule was enteric-coated with ACRYL-EZE® (COLORCON®, Harleysville, PA) prior to delivery via oral gavage to target content delivery to the small intestine.

Bioavailability and pharmacokinetics of ShK-235 after oral delivery. Male rats received a single oral gavage of either 1×10\hat{ }9 cfus LrS235 or an enteric-coated capsule filled with ShK-235. Blood was collected via the saphenous vein at the different time points indicated in the figure, the last blood draw being a terminal cardiac puncture (48). Serum samples were assayed for Kv1.3 block by patch-clamp electrophysiology.

Induction and monitoring of an active delayed-type hypersensitivity reaction. Male and female rats were immunized in the flanks with 200 μl of a 1:1 emulsion of ovalbumin (Sigma, St. Louis, MO) in complete Freund's adjuvant (Difco/Becton Dickinson, Franklin Lakes, NJ)(25). After 7 days, under isoflurane anesthesia, the rats were challenged with ovalbumin dissolved in saline in the pinna of one ear; the collateral ear was injected with saline (49). Rats received either a single subcutaneous injection of 0.1 mg/kg ShK-235 or P6N vehicle, a single oral gavage of 1×10\hat{ }9 cfus LrS235 or LrGusA, oral gavage of 1 ml supernatant of LrS235, or a capsule filled with ShK-235 or gelatin immediately before the ear challenge. The DTH reaction was measured 24 hours post-challenge as the thickness of the ear using a spring-loaded micrometer (Mitutoyo, Japan) and ear inflammation was determined by comparing the ear thickness of the ovalbumin-challenged with the saline-challenged ear from each (49). The animals were euthanized after the ear measurements.

Induction, monitoring, randomization, and treatment of rat collagen-induced arthritis. CIA was induced as described previously (46, 50). Briefly, female Lewis rats received a subcutaneous injection of 200 μl of a 1:1 emulsion of 2 mg/ml porcine type II collagen (20031, Chondrex, Redmond, WA) with incomplete Freund's adjuvant at the base of the tail. After 7 days, rats were given a booster of 100 μl of collagen and adjuvant emulsion. Disease onset was defined as the development of at least one swollen or red paw joint. Clinical scores were determined daily by assigning 1 point for each swollen or red toe joint, 2 points for mildly swollen wrist or ankle joints, and 5 points for each severely swollen wrist or ankle, giving each rat a maximum possible score of 60. Upon disease onset, rats were treated every other day by the subcutaneous injection of P6N buffer vehicle or 0.1 mg/kg ShK-235, or the oral gavage of 1×10\hat{ }9 cfus LrGusA or 1×10\hat{ }9 cfus LrS235 daily. CIA is more severe in rats with early disease onset. To avoid biasing results based on disease severity on the day each rat developed signs of disease and accounting for differences in the time between immunization and when a rat developed signs of illness, every rat that developed signs of disease on a given day was placed in a different treatment group to fill all groups in parallel.

Histology and micro-CT. Healthy rats and rats from the CIA trials were euthanized after 21 days of treatment, and their hind paws were collected and fixed in 10% buffered formalin. One paw from each rat was imaged by Micro-CT using a Bruker SkyScan 1272 Scanner set at 13 μm resolution with no filtering, no averaging, and a rotation step of 0.3. Raw images were analyzed with CTvox (Bruker, MA, US). The other hind paw was decalcified, embedded in paraffin, and sectioned. Slides were stained with either hematoxylin and eosin or Safranin O/Fast green and imaged at 4× magnification on a Nikon Ci-L bright-field microscope (Nikon Inc. NY, US). Scoring of the slides was completed by an investigator blinded to treatment groups using a comprehensive histological scoring system as described elsewhere (46, 51), in which cartilage degradation, cartilage erosion, angiogenesis, pannus formation, synovial hyperplasia, and synovial inflammation were evaluated by the following criteria: 0=absent, 1=mild, 2=moderate, 3=severe. The data were presented as mean±standard deviation.

Immunogenicity assays in rats with CIA. High-protein binding 96-well microplates (3855, ThermoFisher, MA, US) were coated overnight with 10 μg/ml of either porcine collagen II (20031, Chondrex, Woodinville, WI), ShK-235, or HsTX1[R14A], dissolved in PBS. Non-specific binding sites were blocked with PBS+5% skimmed milk and washed with PBS+0.05% Tween 20 before adding serum from the CIA rats, diluted in PBS. After washes, anti-rat IgG antibodies conjugated to horseradish peroxidase (1 μg/ml; Pierce catalog 31471, lot UA280036) were added to all the wells. One-step TMB-ELISA (34028, ThermoFisher, MA, US) was used to detect absorbance on a plate reader at 650 nm.

Antibody neutralization assays. ShK-235 was pre-incubated with media supplemented with 10% of serum from the rats of the CIA trials or healthy, non-immunized and untreated rats. It was then added to the PBMCs loaded with CELLTRACE™ Violet (C34557, ThermoFisher, MA, US), and T cells proliferation was assessed as described above.

Long-term immunogenicity assays in healthy rats. Healthy Lewis rats received a daily oral gavage of 1×10\hat{ }9 cfus LrS235, 1×10\hat{ }9 cfus induced LrGusA, or vehicle for 8 weeks. The rats were left untreated for 12 weeks and then were again treated daily for one week, reaching a total of 21 weeks. Blood was drawn at the 8 week and the 21 week time points and serum was tested for IgG against ShK-235 and HsTX1 [R14A] by ELISA, as described above.

Statistical Analysis. Student's T-test, one-way ANOVA, and two-way ANOVA without correction were used to determine whether differences among the groups were statistically significant (P<0.05). The CIA clinical scores analyses were completed using repeated measure one-way analysis of variance with Bonferroni post hoc test. Data were presented as mean±SEM. All analyses were performed using GraphPad Prism.

All of the methods disclosed and claimed herein can be made and executed without undue experimentation in light of the present disclosure. While the compositions and methods of this disclosure have been described in terms of preferred aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference in their entirety.

### Example 1 References

1. Pennington M W, Chang S C, Chauhan S, Huq R, Tajhya R B, Chhabra S, Norton R S, Beeton C. Development of highly selective Kv1.3-blocking peptides based on the sea anemone peptide ShK. March Drugs 2015; 13:529-542.
2. Beeton C, Chandy K G. Potassium channels, memory T cells and multiple sclerosis. Neuroscientist 2005; 11:550-562.
3. Beeton C, Pennington M W, Norton R S. Analogs of the sea anemone potassium channel blocker ShK for the treatment of autoimmune diseases. Inflamm Allergy Drug Targets 2011; 10:313-321.
4. Chi V, Pennington M W, Norton R S, Tarcha E J, Londono L M, Sims-Fahey B, Upadhyay S K, Lakey J T, Iadonato S, Wulff H, Beeton C, Chandy K G. Development of a sea anemone toxin as an immunomodulator for therapy of autoimmune diseases. Toxicon 2012; 59:529-546.
5. Chandy K G, Norton R S. Peptide blockers of Kv1.3 channels in T cells as therapeutics for autoimmune diseases. Curr Opin Chem Biol 2017; 38:97-107.
6. Beeton C, Pennington M W, Wulff H, Singh S, Nugent D, Crossley G, Khaytin I, Calabresi P A, Chen C Y, Gutman G A, Chandy K G. Targeting effector memory T cells with a selective peptide inhibitor of Kv1.3 channels for therapy of autoimmune diseases. Mol Pharmacol 2005; 67:1369-1381.
7. Beeton C, Wulff H, Singh S, Botsko S, Crossley G, Gutman G A, Cahalan M D, Pennington M W, Chandy K G. A novel fluorescent toxin to detect and investigate Kv1.3 channel up-regulation in chronically activated T lymphocytes. J Biol Chem 2003; 278(11):9928-9937.
8. Beeton C, Wulff H, Standifer N E, Azam P, Mullen K M, Pennington P W, Kolski-Andreaco A, Wei E, Grino A, Counts D R, Wang P H, LeeHealey C J, Andrews B S, Sankaranarayanan A, Homerick D, Roeck W W, Tehranzadeh J, Stanhope K L, Zimin P, Havel P J, Griffey S, Knaus H G, Nepom G T, Gutman G A, Calabresi P A, Chandy K G. Kv1.3 channels are a therapeutic target for T cell-mediated autoimmune diseases. Proc Natl Acad Sci USA 2006; 103:17414-17419.
9. Wulff H, Calabresi P, Allie R, Yun S, Pennington M W, Beeton C, Chandy K G. The voltage-gated Kv1.3 K+ channel in effector memory T cells as new target for MS. J Clin Invest 2003; 111:1703-1713.
10. Fasth A, Cao D, van Vollenhoven R, Trollmo C, Malmstrom V. CD28nullCD4+ T cells—characterization of an effector memory T-cell population in patients with rheumatoid arthritis. Scand J Immunol 2004; 60:199-208.
11. Friedrich M, Krammig S, Henze M, Docke W D, Sterry W, Asadullah K. Flow cytometric characterization of lesional T cells in psoriasis: intracellular cytokine and surface antigen expression indicates an activated, memory/effector type 1 immunophenotype. Arch Dermatol Res 2000; 292:519-521.
12. Lovett-Racke A E, Trotter J L, Lauber J, Perrin P J, June C H, Racke M K. Decreased dependence of myelin basic protein-reactive T cells on CD28-mediated costimulation in multiple sclerosis patients: a marker of activated/memory T cells. J Clin Invest 1998; 101:725-730.
13. Rus H, Pardo C A, Hu L, Darrah E, Cudrici C, Niculescu T, Niculescu F, Mullen K M, Allie R, Gao L, Wulff H, Beeton C, Judge S I V, Kerr D A, Knaus H G, Chandy K G, Calabresi P A. The voltage-gated potassium channel Kv1.3 is highly expressed on inflammatory infiltrates in multiple sclerosis brain. Proc Natl Acad Sci USA 2005; 102:11094-11099.
14. Viglietta V, Kent S C, Orban T, Hafler D A. GAD65-reactive T cells are activated in patients with autoimmune type 1a diabetes. J Clin Invest 2002; 109:895-903.
15. Vissers WHPM, Arndtz C H M, Muys L, Van Erp P E J, De Jong E M G, Van De Kerhof P C M. Memory effector (CD45RO+) and cytotoxic (CD8+) T cells appear early in the margin zone of spreading psoriatic lesions in contrast to cells expressing natural killer receptors, which appear late. Br J Dermatol 2002; 150:852-859.
16. Tarcha E J, Olsen C M, Probst P, Peckham D, Muñoz-Elías E J, Kruger J G, Iadonato S P. Safety and pharmacodynamics of dalazatide, a Kv1.3 channel inhibitor, in the treatment of plaque psoriasis: a randomized phase 1b trial. PLoS ONE 2017; 12:e0180762.
17. Koshy S, Huq R, Tanner M R, Atik M A, Porter P C, Khan F S, Pennington M W, Hanania N A, Corry D B, Beeton C. Blocking Kv1.3 channels inhibits Th2 lymphocyte function and treats a rat model of asthma. J Biol Chem 2014; 289:12623-12632.
18. Munoz-Elias E J, Peckham D, Norton K, Duculan J, Cueto I, Li X, Qin J, Lustig K, Tarcha E, Odegard J, Krueger J G, Iadonato S P. Dalazatide (ShK-186), a first-in-class blocker of Kv1.3 potassium channel on effector memory T cells: safety, tolerability and proof of concept of immunomodulation in patients with active plaque psoriasis Arthritis Rheumatol 2015; 67 Suppl. 10.
19. Beeton C. Targets and therapeutic properties of venom peptides. In: Kastin A J, editor. The handbook of peptides, 2nd edition: Elsevier; 2012. p 473-482.
20. Voskuyl A E. The heart and cardiovascular manifestations in rheumatoid arthritis. Rheumatology 2006; 45, Suppl. 4:iv4-7.
21. van den Bemt B J, Zwikker H E, van den Ende C H. Medication adherence in patients with rheumatoid arthritis: a critical appraisal of the existing literature. Expert Rev Clin Immunol 2012; 8:337-351.
22. Fidder H H, M.M. S, van der Have M, Oldenburg B, van Oijen M G. Low rates of adherence for tumor necrosis factor-α inhibitors in Crohn's disease and rheumatoid arthritis: results of a systematic review. World J Gastroenterol 2013; 19:4344-4450.
23. Sallusto F, Lenig D, Forster R, Lipp M, Lanzavecchia A. Two subsets of memory T lymphocytes with distinct homing potentials and effector functions. Nature 1999; 401(6754):708-712.
24. Gattorno M, Prigione I, Morandi F, Gregorio A, Chiesa S, Ferlito F, Favre A, Uccelli A, Gambini C, Martini A, Pistoia V. Phenotypic and functional characterisation of CCR7+ and CCR7− CD4+ memory T cells homing to the joints in juvenile idiopathic arthritis. Arthritis Res Ther 2005; 7:R256-R267.
25. Burman A, Haworth O, Hardie D L, Amft E N, Sievert C, Jackson D G, Salmon M, Buckley C D. A chemokine-dependent stromal induction mechanism for aberrant lymphocyte accumulation and compromised lymphatic return in rheumatoid arthritis. J Immunol 2005; 174:1693-1700.
26. Markovic-Plese S, Cortese I, Wandinger K P, McFarland H F, Martin R. CD4+CD28− costimulation-independent T cells in multiple sclerosis. J Clin Invest 2001; 108:1185-1194.
27. Davalos-Misslitz A C, Rieckenberg J, Willenzon S, Worbs T, Kremmer E, Bernhardt G, Förster R. Generalized multi-organ autoimmunity in CCR7-deficient mice. Eur J Immunol 2007; 37:613-622.
28. Hille B. Ionic channels of excitable membranes. Sunderland, Mass.: Sinauer Associates; 2001. 426 p.
29. Feske S. Calcium signalling in lymphocyte activation and disease. Nat Rev Immunol 2007; 7:690-702.
30. Panyi G, Vamosi G, Bacso Z, Bagdany M, Bodnar A, Varga Z, Gaspar R, Matyus L, Damjanovich S. Kv1.3 potassium channels are localized in the immunological synapse formed between cytotoxic and target cells. Proc Natl Acad Sci USA 2004; 101:1285-1290.
31. Nicolaou S A, Neumeier L, Steckly A, Kucher V, Takimoto K, Conforti L. Localization of Kv1.3 channels in the immunological synapse modulates the calcium response to antigen stimulation in T lymphocytes. J Immunol 2009; 183:6296-6302.
32. Hanada T, Lin L, Chandy K G, Oh S S, Chisthi A H. Human homologue of the Drosophila discs large tumor suppressor binds to p56lck tyrosine kinase and Shaker type Kv1.3 potassium channel in T lymphocytes. J Biol Chem 1997; 272(43):26899-26904.
33. Matheu M P, Beeton C, Garcia A, Chi V, Rangaraju S, Safrina O, Monaghan K, Uemura M I, Li D, Pal S, de la Maza L M, Monuki E, Flugel A, Pennington M W, Parker I, Chandy K G, Cahalan M D. Imaging of effector memory T cells during a delayed-type hypersensitivity reaction and suppression by Kv1.3 channel block. Immunity 2008; 29(4):602-614.
34. Beeton C, Barbaria J, Giraud P, Devaux J, Benoliel A, Gola M, Sabatier J M, Bernard D, Crest M, Beraud E. Selective blocking of voltage-gated K+ channels improves experimental autoimmune encephalomyelitis and inhibits T cell activation. J Immunol 2001; 166:936-944.
35. Ghanshani S, Wulff H, Miller M J, Rohm H, Neben A, Gutman G A, Cahalan M D, Chandy K G. Upregulation of the IKCal potassium channel during T-cell activation: Molecular Mechanism and functional consequences. J Biol Chem 2000; 275:37137-37149.
36. Tanner M R, Tajhya R B, Huq R, Gehrmann E J, Rodarte K E, Atik M A, Norton R S, Pennington M W, Beeton C. Prolonged immunomodulation in inflammatory arthritis using the selective Kv1.3 channel blocker HsTX1[R14A] and its PEGylated analog. Clin Immunol 2017; 180:45-57.
37. Tarcha E J, Chi V, Munoz-Elias E, Bailey D, Londono L M, Upadhyay S K, Norton K, Olson A, Tjong I, Nguyen H, Hu X, Rupert G W, Boley S E, Slauter R, Sams J, Knapp B, Pennington M W, Beeton C, Chandy K G, Iadonato S P. Durable pharmacological responses from the peptide ShK-186, a specific Kv1.3 channel inhibitor that suppresses T cell mediators of autoimmune diseases. J Pharmacol Exp Ther 2012; 342:642-653.
38. Pereira L E, Villinger F, Wulff H, Sankaranarayanan A, Raman G, Ansari A A. Pharmacokinetics, toxicity, and functional studies of the selective Kv1.3 channel blocker 5-(4-phenoxybutoxy)psoralen in rhesus macaques. Exp Biol Med (Maywood) 2007; 232:1338-1354.
39. Chandy K G, Beeton C, Pennington M W; Analogs of ShK toxin and their uses in selective inhibition of Kv1.3 potassium channels. 2006.
40. Tanner M R, Pennington M W, Chauhan S S, Laragione T, Gulko P S, Beeton C. KCa1.1 and Kv1.3 channels regulate the interactions between fibroblast-like synoviocytes and T lymphocytes during rheumatoid arthritis. Arthritis Res Ther 2019; 21:6.
41. Chang S C, Galea C A, Leung E W W, Tajhya R B, Beeton C, Norton R S. Expression and isotopic labeling of the potassium channel blocker ShK toxin as a thioredoxin fusion protein in bacteria. Toxicon 2012; 60:840-850.
42. Anangi R, Koshy S, Huq R, Beeton C, Chuang W J, King G F. Recombinant expression of margatoxin and agitoxin-2 in Pichia pastoris: an efficient method for production of KV1.3 channel blockers. PLoS ONE 2012; 7:e52965.
43. Chang S C, Huq R, Chhabra S, Beeton C, Pennington M W, Smith B J, Norton R S. N-terminally-extended analogues of the K+ channel toxin from Stichodactyla helianthus as potent and selective blockers of the voltage-gated potassium channel Kv1.3. FEBS J 2015; 282:2247-2259.
44. Collins F L, Irwin R, Bierhalter H, Schepper J, Britton R A, Parameswaran N, McCabe L R. Lactobacillus reuteri 6475 increases bone density in intact females only under an inflammatory setting. PLoS ONE 2016; 11:e0153180.
45. Eaton K A, Honkala A, Auchtung T A, Britton R A. Probiotic Lactobacillus reuteri ameliorates disease due to enterohemorrhagic Escherichia coli in germfree mice. Infect Immun 2011; 79:185-191.
46. Ortiz-Velez L, Ortiz-Villalobos J, Schulman A, Oh J H, van Pijkeren J P, Britton R A. Genome alterations associated with improved transformation efficiency in Lactobacillus reuteri. Microb Cell Fact 2018; 17:138.
47. Beeton C, Smith B J, Sabo J K, Crossley G, Nugent D, Khaytin I, Chi V, Chandy K G, Pennington M W, Norton R S. The D-diastereoisomer of ShK toxin selectively blocks voltage-gated K+ channels and inhibits T lymphocyte proliferation. J Biol Chem 2008; 283:988-997.
48. Beeton C, Wulff H, Barbaria J, Clot-Faybesse O, Pennington M W, Bernard D, Cahalan M D, Chandy K G, Beraud E. Selective blockade of T lymphocyte K+ channels ameliorates experimental autoimmune encephalomyelitis, a model for multiple sclerosis. Proc Natl Acad Sci USA 2001; 98:13942-13947.
49. Pennington M W, Beeton C, Galea C A, Smith B J, Chi V, Monaghan K P, Garcia A, Rangaraju S, Giuffrida A, Plank D, Crossley G, Nugent D, Khaytin I, LeFievre Y, Peshenko I, Dixon C, Chauhan S, Orzel A, Inoue T, Hu X, Moore R V, Norton R S, Chandy K G. Engineering a stable and selective peptide blocker of the Kv1.3 channel in T lymphocytes. Mol Pharmacol 2009; 75:762-773.
50. Walker U A, Courvoisier D S, Dudler J, Aeberli D, von Kempis J, Scherer A, Finckh A, Swiss Clinical Quality Management Programme in Rheumatic Diseases. Do new biologics meet the unmet medical need in rheumatoid arthritis? Safety and efficacy of abatacept following B-cell depletion. Rheumatology (Oxford) 2011; 50:243-244.
51. Bijlsma J W. Optimal treatment of rheumatoid arthritis: EULAR recommendations for clinical practice. Pol Arch Med Wewn 2010; 120:347-353.
52. Chaabo K, Kirkham B. Rheumatoid Arthritis—Anti-TNF. Int Immunopharmacol 2015; 27:180-184.
53. Colmegna I, Ohatra B R, Menard H A. Current Understanding of Rheumatoid Arthritis Therapy. Clin Pharmacol Ther 2012; 91:607-620.
54. Gibofsky A. Comparative effectiveness of current treatments for rheumatoid arthritis. Am J Manag Care 2012; 13 Suppl:S303-314.
55. Gibofsky A. Current therapeutic agents and treatment paradigms for the management of rheumatoid arthritis. Am J Manag Care 2014; 20 (7 Suppl):S136-144.
56. Murray E, Ellis A, Butylkova Y, Skup M, Kalabic J, Garg V. Systematic review and network metaanalysis: effect of biologics on radiographic progression in rheumatoid arthritis. J Comp Eff Res 2018; [Epub ahead of print].
57. Wong P K. Medication adherence in patients with rheumatoid arthritis: why do patients not take what we prescribe? Rheumatol Int 2016; 11:1535-1542.
58. Gomes-Santos A C, de Oliveira R P, Moreira T G, Castro-Junior A B, Horta B C, Lemos L, de Almeida L A, Rezende R M, Cara D C, Oliveira S C, Azevedo V A, Miyoshi A, Faria A M. Hsp65-producing Lactococcus lactis prevents inflammatory intestinal disease in mice by IL-10- and TLR2-dependent pathways. Front Immunol 2017; 8:30.
59. Souza B M, Preisser T M, Pereira V B, Zurita-Turk M, de Castro C P, da Cunha V P, de Oliveira R P, Gomes-Santos A C, de Faria A M, Machado D C, Chatel J M, Azevedo V A, Langella P, Miyoshi A. Lactococcus lactis carrying the pValac eukaryotic expression vector coding for IL-4 reduces chemically-induced intestinal inflammation by increasing the levels of IL-10-producing regulatory cells. Microb Cell Fact 2016; 15:150.
60. Chhabra S, Chang S C, Nguyen H M, Huq R, Tanner M R, Londono L M, Estrada R, Dhawan V, Chauhan S, Upadhyay S K, Gindin M, Hotez P J, Valenzuela J G, Mohanty B, Swarbrick J D, Wulff H, Iadonato S P, Gutman G A, Beeton C, Pennington M W, Norton R S, Chandy K G. Kv1.3 channel-blocking immunomodulatory peptides from parasitic worms: implications for autoimmune diseases. FASEB J 2014; 28:3952-3964.
61. Pennington M W, Rashid M H, Tajhya R B, Beeton C, Kuyucak S, Norton R S. A C-terminally amidated analogue of ShK is a potent and selective blocker of the voltage-gated potassium channel Kv1.3. FEBS Lett 2012; 586:3996-4001.
62. Rashid M H, Heinzelmann G, Huq R, Tajhya R B, Chang S C, Chhabra S, Pennington M W, Beeton C, Norton R S, Kuyucak S. A potent and selective peptide blocker of the Kv1.3 channel: prediction from free-energy simulations and experimental confirmation. PLoS ONE 2013; 8:e78712.
63. Rashid M H, Huq R, Tanner M R, Chhabra S, Khoo K K, Estrada R, Dhawan V, Chauhan S, Pennington M W, Beeton C, Kuyucak S, Norton R S. A potent and Kv1.3-selective analogue of the scorpion toxin HsTX1 as a potential therapeutic for autoimmune diseases. Sci Rep 2014; 4:4509.
64. Varga Z, Gurrola-Briones G, Papp F, Rodriguez de la Vega R C, Pedraza-Alva G, Tajhya R B, Gaspar R, Cardenas L, Rosenstein Y, Beeton C, Possani L D, Panyi G. Vm24, a natural immunosuppressant peptide potently and selectively blocks Kv1.3 potassium channels of human T cells. Mol Pharmacol 2012; 82:372-382.
65. Tanner M R, Hu X, Huq R, Tajhya R B, Sun L, Khan F S, Laragione T, Horrigan F T, Gulko P S, Beeton C. KCa1.1 inhibition attenuates fibroblast-like synoviocyte invasiveness and ameliorates rat models of rheumatoid arthritis. Arthritis Rheumatol 2015; 67:96-106.
66. Tanner M R, Pennington M W, Chamberlain B H, Huq R, Gehrmann E J, Laragione T, Gulko P S, Beeton C. Targeting KCa1.1 channels with a scorpion venom peptide for the therapy of rat models of rheumatoid arthritis. J Pharmacol Exp Ther 2018; 365:227-236.
67. Seo N S, Zeng C Q Y, Hyser J M, Utama B, Crawford S E, Kim K J, Höök M, Estes M K. Integrins α1β1 and α2β1 are receptors for the rotavirus enterotoxin. Proc Natl Acad Sci USA 2008; 105:8811-8818.
68. Crawford S E, Patel D G, Cheng E, Berkova Z, Hyser J M, Ciarlet M, Finegold M J, Conner M E, Estes M K. Rotavirus viremia and extraintestinal viral infection in the neonatal rat model. J Virol 2006; 80:4820-4832.
69. Foulke-Abel J, In J, Kovbasnjuk O, Zachos N C, Ettayebi K, Blutt S E, Hyser J M, Zeng X L, Crawford S E, Broughman J R, Estes M K, Donowitz M. Human enteroids as an ex-vivo model of host-pathogen interactions in the gastrointestinal tract. Exp Biol Med (Maywood) 2014; 239:1124-1134.
70. van Pijkeren J P, Britton R A. High efficiency recombineering in lactic acid bacteria. Nucleic Acids Res 2012; 40:e76.
71. Van Pijkeren J P, Britton R A. Precision genome engineering in lactic acid bacteria. Microb Cell Fact 2014; 13 Suppl 1:S10.
72. Britton R A, Irwin R, Quach D, Schaefer L, Zhang J, Lee T, Parameswaran N, McCabe L R. Probiotic L. reuteri treatment prevents bone loss in a menopausal ovariectomized mouse model. J Cell Physiol 2014; 229:1822-1830.
73. Norton R S, Pennington M W, Beeton C. Transforming a toxin into a therapeutic: the sea anemone potassium channel blocker ShK toxin for treatment of autoimmune diseases. In: King G F, editor. Venoms to drugs: Venom as a source for the development of human therapeutics: Royal Society of Chemistry; 2015. p 255-274.
74. Tanner M R, Beeton C. Differences in ion channel phenotype and function between humans and animal models. Front Biosci 2018; 23:43-64.
75. Tudor J E, Pennington M W, Norton R S. Ionisation behaviour and solution properties of the potassium-channel blocker ShK toxin. Eur J Biochem 1998; 251(1-2):133-141.
76. Azam P, Sankaranarayanan A, Homerick D, Griffey S, Wulff H. Targeting effector memory T cells with the small molecule Kv1.3 blocker PAP-1 suppresses allergic contact dermatitis. J Invest Dermatol 2007; 127:1419-1429.
77. Gocke A R, Lebson L A, Grishkan I V, Hu L, Nguyen H M, Whartenby K A, Chandy K G, Calabresi P A. Kv1.3 deletion biases T cells toward an immunoregulatory phenotype and renders mice resistant to autoimmune encephalomyelitis. J Immunol 2012; 188:5877-5886.
78. Bergmann R, Kubeil M, Zarschler K, Chhabra S, Tajhya R B, Beeton C, Pennington M W, Bachmann M, Norton R S, Stephan H. Distribution and kinetics of the Kv1.3-blocking peptide HsTX1[R14A] in experimental rats. Sci Rep 2017; 7:3756.
79. Cook Sangar M L, Girard E J, Hopping G, Yin C, Paklam F, Brusniak M Y, Nguyen E, Ruff R, Gewe M M, Byrnes-Blake K, Nairn N W, Miller D M, Mehlin C, Strand A D, Mhyre A J, Correnti C E, Strong R K, Simon J A, Olson J M. A potent peptide-steroid conjugate accumulates in cartilage and reverses arthritis without evidence of systemic corticosteroid exposure. Sci Transl Med 2020; 12:eaay1041.
80. Ong S, Bajaj S, Tanner M R, Chang S, Krishnarjuna B, Ng X, Morales R A, Chen M, Luo D, Patel D, Yasmin S, Ng J J, Zhuang Z, Nguyen H M, El Sahili A, Lescar J, Patil R, Charman S A, Robbins E G, Goggi J L, Tan P, Sadasivam P, Ramasamy B, Hartimath S V, Dhawan V, Bednenko J, Colussi P, Wulff H, Pennington M W, Kuyucak S, Norton R S, Beeton C, Chandy K G. Modulation of lymphocyte potassium channel KV1.3 by membrane-penetrating, joint-targeting immunomodulatory plant defensin. ACS Pharmacol Transl Sci 2020; 3:720-736.
81. Hume W J, Keat S. Immunohistological optimization of detection of bromodeoxyuridine-labeled cells in decalcified tissue. J Histochem Cytochem 1990; 38:509-513.
82. Sakuma M, Hatsushika K, Koyama K, Katoh R, Ando T, Watanabe Y, Wako M, Kanzaki M, Takano S, Sugiyama H, Hamada Y, Ogawa H, Okumura K, Nakao A. TGF-beta type I receptor kinase inhibitor down-regulates rheumatoid synoviocytes and prevents the arthritis induced by type II collagen antibody. Int Immunol 2007; 19:117-126.
83. Taneja V. Arthritis susceptibility and the gut microbiome. FEBS Lett 2014; 588:4244-4249.
84. Sato K, Takahashi N, Kato T, Matsuda Y, Yokoji M, Yamada M, Nakajima T, Kondo N, Endo N, Yamamoto R, Noiri Y, Ohno H, Yamazaki K. Aggravation of collagen-induced arthritis by orally administered Porphyromonas gingivalis through modulation of the gut microbiota and gut immune system. Sci Rep 2017; 7:6955.
85. Maeda Y, Takeda K. Role of gut microbiota in rheumatoid arthritis. J Clin Med 2017; 6:E60.
86. Jubair W K, Hendrickson J D, Severs E L, Schulz H M, Adhikari S, Ir D, Pagan J D, Anthony R M, Robertson C E, Frank D N, Banda N K, Kuhn K A. Modulation of inflammatory arthritis in mice by gut microbiota through mucosal inflammation and autoantibody generation. Arthritis Rheum 2018; 70:1220-1233.
87. Abroug F, Ouanes-Besbes L, Ouanes I, Dachraoui F, Hassen M F, Haguiga H, Elatrous S, Brun-Buisson C. Meta-analysis of controlled studies on immunotherapy in severe scorpion envenomation. Emerg Med J 2011; 28:963-969.
88. Hougaard Laustsen A, Sold M, Jappe E C, Oscoz S, Lauridsen L P, Engmark M. Biotechnological Trends in Spider and Scorpion Antivenom Development. Toxins (Basel) 2016; 8:226.
89. Rangaraju S, Khoo K K, Feng Z P, Crossley G, Nugent D, Khaytin I, Chi V, Pham C, Calabresi P, Pennington M W, Norton R S, Chandy K G. Potassium channel modulation by a toxin domain in matrix metalloprotease 23. J Biol Chem 2010; 285:9124-9136.
90. Galea C A, Nguyen H M, Chandy K G, Smith B J, Norton R S. Domain structure and function of matrix metalloprotease 23 (MMP23): role in potassium channel trafficking. Cell Mol Life Sci 2014; 71:1191-1210.
91. Grissmer S, Nguyen A N, Aiyar J, Hanson D C, Mather R J, Gutman G A, Karmilowicz M J, Auperin D D, Chandy K G. Pharmacological characterization of five cloned voltage-gated K+ channels, types Kv1.1, 1.2, 1.3, 1.5, and 3.1, stably expressed in mammalian cell lines. Mol Pharmacol 1994; 45:1227-1234.
92. Huq R, Samuel E L, Sikkema W K, Nilewski L G, Lee T, Tanner M R, Khan F S, Porter P C, Tajhya R B, Patel R S, Inoue T, Pautler R G, Corry D B, Tour J M, Beeton C. Preferential uptake of antioxidant carbon nanoparticles by T lymphocytes for immunomodulation. Sci Rep 2016; 6:33808.
93. Beeton C, Chandy K G. Drawing blood from the saphenous vein of rats and by cardiac puncture. J Vis Exp 2007; 7:e266.
94. Oh J H, van Pijkeren J P. CRISPR-Cas9-assisted recombineering in Lactobacillus reuteri. Nucleic Acids Res 2014; 42:e131.
95. Zhang S, Oh J H, Alexander L M, Ozgam M, van Pijkeren J P. d-Alanyl-d-Alanine Ligase as a Broad-Host-Range Counterselection Marker in Vancomycin-Resistant Lactic Acid Bacteria. J Bacteriol 2018; 200:e00607-00617.
96. Sonoyama K, Miki A, Sugita R, Goto H, Nakata M, Yamaguchi N. Gut colonization by Candida albicans aggravates inflammation in the gut and extra-gut tissues in mice. Medical Mycology 2011; 49:237-247.
97. Auchtung J M, Robinson C D, Britton R A. Cultivation of stable, reproducible microbial communities from different fecal donors using minibioreactor arrays (MBRAs). Microbiome 2015; 3:42.
98. Auchtung J M, Robinson C D, Farrell K, Britton R A. MiniBioReactor Arrays (MBRAs) as a tool for studying C. difficile physiology in the presence of a complex community. Methods Mol Biol 2016; 1476:235-258.
99. Zhao R, Dai H, Mendelman N, Cuello L G, Chill J H, Goldstein S A N. Designer and natural peptide toxin blockers of the KcsA potassium channel identified by phage display. Proc Natl Acad Sci USA 2015; 112:E7013-E7021.

### Example 2 References

1. Boscarino J A, Forsberg C W, Goldberg J. A twin study of the association between PTSD symptoms and rheumatoid arthritis. Psychosom Med 2010; 72:481-486.
2. O'Donovan A, Cohen B E, Seal K H, Bertenthal D, Margaretten M, Nishimi K, Neylan T C. Elevated risk for autoimmune disorders in Iraq and Afghanistan veterans with posttraumatic stress disorder. Biol Psychiatry 2015; 77:365-374.
3. Voskuyl A E. The heart and cardiovascular manifestations in rheumatoid arthritis. Rheumatology 2006; 45, Suppl. 4:iv4-7.
4. van den Bemt B J, Zwikker H E, van den Ende C H. Medication adherence in patients with rheumatoid arthritis: a critical appraisal of the existing literature. Expert Rev Clin Immunol 2012; 8:337-351.
5. Bottini N, Firestein G S. Duality of fibroblast-like synoviocytes in RA: passive responders and imprinted aggressors. Nat Rev Rheumatol 2013; 9:24-33.
6. Litan A, Langhans S A. Cancer as a channelopathy: ion channels and pumps in tumor development and progression. Front Cell Neurosci 2015; 9:86.
7. Hu X, Laragione T, Sun L, Koshy S, Jones K R, Ismailov I I, Yotnda P, Horrigan F T, Gulko P S, Beeton C. KCa1.1 potassium channels regulate key pro-inflammatory and invasive properties of fibroblast-like synoviocytes in rheumatoid arthritis. J Biol Chem 2012; 287:4014-4022.
8. Tanner M R, Hu X, Huq R, Tajhya R B, Sun L, Khan F S, Laragione T, Horrigan F T, Gulko P S, Beeton C. KCa1.1 inhibition attenuates fibroblast-like synoviocyte invasiveness and ameliorates rat models of rheumatoid arthritis. Arthritis Rheumatol 2015; 67:96-106.
9. Haidar O, O'Neill N O, Staunton C A, Bavan S, O'Brien F, Zouggari S, Sharif U, Mobasheri A, Kumagai K, Barrett-Jolley R. Pro-inflammatory cytokines drive deregulation of potassium channel expression in primary synovial fibroblasts. Front Physiol 2020; 11:226.
10. Beeton C. KCa1.1 channels as therapeutic targets for rheumatoid arthritis. Expert Opin Ther Targets 2017; 21:1077-1081.
11. Tanner M R, Pennington M W, Chamberlain B H, Huq R, Gehrmann E J, Laragione T, Gulko P S, Beeton C. Targeting KCa1.1 channels with a scorpion venom peptide for the therapy of rat models of rheumatoid arthritis. J Pharmacol Exp Ther 2018; 365:227-236.
12. Chang S C, Galea C A, Leung E W W, Tajhya R B, Beeton C, Norton R S. Expression and isotopic labeling of the potassium channel blocker ShK toxin as a thioredoxin fusion protein in bacteria. Toxicon 2012; 60:840-850.
13. Collins F L, Irwin R, Bierhalter H, Schepper J, Britton R A, Parameswaran N, McCabe L R. Lactobacillus reuteri 6475 increases bone density in intact females only under an inflammatory setting. PLoS ONE 2016; 11:e0153180.
14. Ortiz-Velez L, Ortiz-Villalobos J, Schulman A, Oh J H, van Pijkeren J P, Britton R A. Genome alterations associated with improved transformation efficiency in Lactobacillus reuteri. Microb Cell Fact 2018; 17:138.
15. Choi J H, Moon C M, Shin T-S, Kim E K, McDowell A, Jo M-K, Joo Y S, Kim S-E, Jung H-K, Shim K-N, Jung S-A, Kim Y-K. Lactobacillus paracasei-derived extracellular vesicles attenuate the intestinal inflammatory response by augmenting the endoplasmic reticulum stress pathway. Exp Mol Med 2020; 52:423-437.
16. Beeton C. Targets and therapeutic properties of venom peptides. In: Kastin A J, editor. The handbook of peptides, 2nd edition: Elsevier; 2012. p 473-482.
17. Noss E H, Brenner M B. The role and therapeutic implications of fibroblast-like synoviocytes in inflammation and cartilage erosion in rheumatoid arthritis. Immunol Rev 2008; 223:252-270.
18. Bartok B, Firestein G S. Fibroblast-like synoviocytes: key effector cells in rheumatoid arthritis. Immunol Rev 2010; 233:233-255.
19. Filer A. The fibroblast as a therapeutic target in rheumatoid arthritis. Curr Opin Pharmacol 2013; 13:413-419.
20. De Bari C. Are mesenchymal stem cells in rheumatoid arthritis the good or bad guys? Arthritis Res Ther 2015; 17:113.
21. Turner J D, Filer A. The role of the synovial fibroblast in rheumatoid arthritis pathogenesis. Curr Opin Rheumatol 2015; 27:175-182.
22. Lefevre S, Meier F M, Neumann E, Muller-Ladner U. Role of synovial fibroblasts in rheumatoid arthritis. Curr Pharm Res 2015; 21:130-141.
23. Kaczorowski G J, McManus O B, Priest B T, Garcia M L. Ion channels as drug targets: the next GPCRs. J Gen Physiol 2008; 131:399-405.
24. Kochukov M Y, NMcnearney T A, Fu Y, Westlund K N. Thermosensitive TRP ion channels mediate cytosolic calcium response in human synoviocytes. Am J Physiol Cell Physiol 2006; 291:C424-C432.
25. Laragione T, Cheng K F, Tanner M R, He M, Beeton C, Al-Abed Y, Gulko P S. The cation channel Trpv2 is a new suppressor of arthritis severity, joint damage, and synovial fibroblast invasion. Clin Immunol 2015; 158:183-192.
26. Ciurtin C, Majeed Y, Naylor C, Sukumar P, English A E, Emery P, Beech D J. TRPM3 channel stimulated by pregnenolone sulphate in synovial fibroblasts and negatively coupled to hyaluronan. BMC Musculoskeletal Disorders 2010; 11:111.
27. Tanner M R, Pennington M W, Chauhan S S, Laragione T, Gulko P S, Beeton C. KCa1.1 and Kv1.3 channels regulate the interactions between fibroblast-like synoviocytes and T lymphocytes during rheumatoid arthritis. Arthritis Res Ther 2019; 21:6.
28. Contreras G F, Castillo K, Enrique N, Carrasquel-Ursulaez W, Castillo J P, Milesi V, Neely A, Alvarez O, Ferreira G, González C, Latorre R. A BK (Slo1) channel journey from molecule to physiology. Channels (Austin) 2013; 7:442-458.
29. Contreras G F, Neely A, Alvarez O, Gonzalez C, Latorre R. Modulation of BK channel voltage gating by different auxiliary β subunits. Proc Natl Acad Sci USA 2012; 109:18991-18996.
30. Orio P, Rojas P, Ferreira G, Latorre R. New disguises for an old channel: MaxiK channel β-subunits. News Physiol Sci 2002; 17:156-161.
31. Meera P, Wallner M, Toro L. A neuronal beta subunit (KCNMB4) makes the large conductance, voltage- and Ca²⁺-activated K⁺ channel resistant to charybdotoxin and
iberiotoxin. Proc Natl Acad Sci USA 2000; 97:5562-5567.
32. Candia S, Garcia M L, Latorre R. Mode of action of iberiotoxin, a potent blocker of the large conductance Ca²⁺-activated K⁺ channel. Biophys J 1992; 63:583-590.
33. Zhou Y, Lingle C J. Paxilline inhibits BK channels by an almost exclusively closed-channel block mechanism. J Gen Physiol 2014; 144:415-440.
34. Strøbaek D, Christophersen P, Holm N R, Moldt P, Ahring P K, Johansen T E, Olesen S P. Modulation of the Ca²⁺-dependent K⁺ channel, hslo, by the substituted diphenylurea NS 1608, paxilline and internal Ca²⁺. Neuropharmacol 1996; 35(7):903-914.
35. Bukiya A N, Liu J, Toro L, Dopico A M. β1 (KCNMB1) subunits mediate lithocholate activation of large-conductance Ca²⁺-activated K⁺ channels and dilation in small, resistance-size arteries. Mol Pharmacol 2007; 72:359-369.
36. Bukiya A N, McMillan J, Parrill A L, Dopico A M. Structural determinants of monohydroxylated bile acids to activate beta 1 subunit-containing BK channels. J Lipid Res 2008; 49(11):2441-2451.
37. Bukiya A N, Vaithianathan T, Toro L, Dopico A M. Channel β2-4 subunits fail to substitute for β1 in sensitizing BK channels to lithocholate. Biochem Biophys Res Commun 2009; 390:995-1000.
38. Sun X, Zhou D, Zhang P, Moczydlowski E G, Haddad G G. β-subunit-dependent modulation of hslo BK current by arachidonic acid. J Neurophysiol 2007; 97:62-69.
39. Gessner G, Cui Y-M, Otani Y, Ohwada T, Soom M, Hoshi T, Heinemann S H. Molecular mechanism of pharmacological activation of BK channels. Proc Natl Acad Sci USA 2012; 109:3552-3557.
40. Pethő Z, Tanner M R, Tajhya R B, Huq R, Laragione T, Panyi G, Gulko P S, Beeton C. Different expression of β subunits of the KCa1.1 channel by invasive and non-invasive human fibroblast-like synoviocytes. Arthritis Res Ther 2016; 18:103.
41. Anangi R, Koshy S, Huq R, Beeton C, Chuang W J, King G F. Recombinant expression of margatoxin and agitoxin-2 in Pichia pastoris: an efficient method for production of KV1.3 channel blockers. PLoS ONE 2012; 7:e52965.
42. Chang S C, Huq R, Chhabra S, Beeton C, Pennington M W, Smith B J, Norton R S. N-terminally-extended analogues of the K⁺ channel toxin from Stichodactyla helianthus as potent and selective blockers of the voltage-gated potassium channel Kv1.3. FEBS J 2015; 282:2247-2259.
43. Eaton K A, Honkala A, Auchtung T A, Britton R A. Probiotic Lactobacillus reuteri ameliorates disease due to enterohemorrhagic Escherichia coli in germfree mice. Infect Immun 2011; 79:185-191.
44. Xie Z, Zhao Y, Yang W, Li W, Wu Y, Chen Z. Methotrexate, a small molecular scaffold targeting Kv1.3 channel extracellular pore region. Biochem Biophys Res Commun 2020; In press.
45. McGivern J G. Ziconotide: a review of its pharmacology and use in the treatment of pain. Neuropsychiatr Dis Treat 2007; 3:69-85.
46. Nauck M A, Quast D R, Wefers J, Meier J J. GLP-1 receptor agonists in the treatment of type 2 diabetes—state-of-the-art. Mol Metab 2021; 46:101102.
47. Chandy K G, Beeton C, Pennington M W; Analogs of ShK toxin and their uses in selective inhibition of Kv1.3 potassium channels. 2006.
48. Norton R S, Kuyucak S, Rashid H M, Pennington M W, Beeton C; Scorpion toxin analogue and method for treating autoimmune diseases. 2017.
49. Walker U A, Courvoisier D S, Dudler J, Aeberli D, von Kempis J, Scherer A, Finckh A, Swiss Clinical Quality Management Programme in Rheumatic Diseases. Do new biologics meet the unmet medical need in rheumatoid arthritis? Safety and efficacy of abatacept following B-cell depletion. Rheumatology (Oxford) 2011; 50:243-244.
50. Bijlsma J W. Optimal treatment of rheumatoid arthritis: EULAR recommendations for clinical practice. Pol Arch Med Wewn 2010; 120:347-353.
51. Chaabo K, Kirkham B. Rheumatoid Arthritis—Anti-TNF. Int Immunopharmacol 2015; 27:180-184.
52. Colmegna I, Ohatra B R, Menard H A. Current Understanding of Rheumatoid Arthritis Therapy. Clin Pharmacol Ther 2012; 91:607-620.
53. Gibofsky A. Comparative effectiveness of current treatments for rheumatoid arthritis. Am J Manag Care 2012; 13 Suppl:S303-314.
54. Gibofsky A. Current therapeutic agents and treatment paradigms for the management of rheumatoid arthritis. Am J Manag Care 2014; 20 (7 Suppl):S136-144.
55. Murray E, Ellis A, Butylkova Y, Skup M, Kalabic J, Garg V. Systematic review and network meta-analysis: effect of biologics on radiographic progression in rheumatoid arthritis. J Comp Eff Res 2018; [Epub ahead of print].
56. Wong P K. Medication adherence in patients with rheumatoid arthritis: why do patients not take what we prescribe? Rheumatol Int 2016; 11:1535-1542.
57. Gomes-Santos A C, de Oliveira R P, Moreira T G, Castro-Junior A B, Horta B C, Lemos L, de Almeida L A, Rezende R M, Cara D C, Oliveira S C, Azevedo V A, Miyoshi A, Faria A M. Hsp65-producing Lactococcus lactis prevents inflammatory intestinal disease in mice by IL-10- and TLR2-dependent pathways. Front Immunol 2017; 8:30.
58. Souza B M, Preisser T M, Pereira V B, Zurita-Turk M, de Castro C P, da Cunha V P, de Oliveira R P, Gomes-Santos A C, de Faria A M, Machado D C, Chatel J M, Azevedo V A, Langella P, Miyoshi A. Lactococcus lactis carrying the pValac eukaryotic expression vector coding for IL-4 reduces chemically-induced intestinal inflammation by increasing the levels of IL-10-producing regulatory cells. Microb Cell Fact 2016; 15:150.
59. Tanner M R, Pennington M W, Laragione T, Gulko P S, Beeton C. KCa1.1 channels regulate β1 integrin function and cell adhesion in rheumatoid arthritis fibroblast-like synoviocytes. FASEB J 2017; 8:3309-3320.
60. van Pijkeren J P, Britton R A. High efficiency recombineering in lactic acid bacteria. Nucleic Acids Res 2012; 40:e76.
61. Van Pijkeren J P, Britton R A. Precision genome engineering in lactic acid bacteria. Microb Cell Fact 2014; 13 Suppl 1:S10.
62. Britton R A, Irwin R, Quach D, Schaefer L, Zhang J, Lee T, Parameswaran N, McCabe L R. Probiotic L. reuteri treatment prevents bone loss in a menopausal ovariectomized mouse model. J Cell Physiol 2014; 229:1822-1830.
63. Mullmann T J, Munujos P, Garcia M L, Giangiacomo K M. Electrostatic mutations in iberiotoxin as a unique tool for probing the electrostatic structure of the Maxi-K channel outer vestibule. Biochem 1999; 38:2395-2402.

### Example 3 & Additional References

1. A. C. Anselmo, Y. Gokarn, S. Mitragotri, Non-invasive delivery strategies for biologics. Nat Rev Drug Discov 18, 19-40 (2019).
2. J. S. Smolen, D. Aletaha, I. B. McInnes, Rheumatoid arthritis. The Lancet 388, 2023-2038 (2016).
3. A. E. Voskuyl, The heart and cardiovascular manifestations in rheumatoid arthritis. Rheumatology 45, Suppl. 4, iv4-7 (2006).
4. B. J. van den Bemt, H. E. Zwikker, C. H. van den Ende, Medication adherence in patients with rheumatoid arthritis: a critical appraisal of the existing literature. Expert Rev. Clin. Immunol. 8, 337-351 (2012).
5. H. H. Fidder, S. M. M., M. van der Have, B. Oldenburg, M. G. van Oijen, Low rates of adherence for tumor necrosis factor-α inhibitors in Crohn's disease and rheumatoid arthritis: results of a systematic review. World J. Gastroenterol. 19, 4344-4450 (2013).
6. V. Strand, J. Goncalves, J. D. Isaacs, Immunogenicity of biologic agents in rheumatology. Nat. Rev. Rheumatol. 17, 81-97 (2020).
7. S. Atiqi, F. Hooijberg, F. C. Loeff, T. Rispens, G. J. Wolbink, Immunogenicity of TNF-inhibitors. Front. Immunol. 11, 312 (2020).
8. M. Gattorno et al., Phenotypic and functional characterisation of CCR7+ and CCR7− CD4+ memory T cells homing to the joints in juvenile idiopathic arthritis. Arthritis Res. Ther. 7, R256-R267 (2005).
9. C. Beeton et al., Kv1.3 channels are a therapeutic target for T cell-mediated autoimmune diseases. Proc. Natl. Acad. Sci. USA. 103, 17414-17419 (2006).
10. C. Beeton, M. W. Pennington, R. S. Norton, Analogs of the sea anemone potassium channel blocker ShK for the treatment of autoimmune diseases. Inflamm. Allergy Drug Targets 10, 313-321 (2011).
11. V. Chi et al., Development of a sea anemone toxin as an immunomodulator for therapy of autoimmune diseases. Toxicon 59, 529-546 (2012).
12. K. G. Chandy, R. S. Norton, Peptide blockers of Kv1.3 channels in T cells as therapeutics for autoimmune diseases. Curr. Opin. Chem. Biol. 38, 97-107 (2017).
13. E. J. Munoz-Elias et al., Dalazatide (ShK-186), a first-in-class blocker of Kv1.3 potassium channel on effector memory T cells: safety, tolerability and proof of concept of immunomodulation in patients with active plaque psoriasis Arthritis Rheumatol. 67 Suppl. 10 (2015).
14. E. J. Tarcha et al., Safety and pharmacodynamics of dalazatide, a Kv1.3 channel inhibitor, in the treatment of plaque psoriasis: a randomized phase 1b trial. PLoS ONE 12, e0180762 (2017).
15. L. Jin et al., Enabling Noninvasive Systemic Delivery of the Kv1.3-Blocking Peptide HsTX1[R14A] via the Buccal Mucosa. J Pharm Sci 105, 2173-2179 (2016).
16. M. W. Pennington et al., Development of highly selective Kv1.3-blocking peptides based on the sea anemone peptide ShK. March Drugs 13, 529-542 (2015).
17. i. b. Food and Agriculture Organization of the United Nations, i. b. World Health Organization (2006) Probiotics in food: health and nutritional properties and guidelines for evaluation. (Rome: World Health Organization: Food and Agriculture Organization of the United Nations, 2006, Italy).
18. Q. Mu, V. J. Tavella, X. M. Luo, Role of Lactobacillus reuteri in Human Health and Diseases. Front Microbiol 9, 757 (2018).
19. F. L. Collins et al., Lactobacillus reuteri 6475 increases bone density in intact females only under an inflammatory setting. PLoS ONE 11, e0153180 (2016).
20. M. van Asseldonk, W. M. de Vos, G. Simons, Functional analysis of the Lactococcus lactis usp45 secretion signal in the secretion of a homologous proteinase and a heterologous alpha-amylase. Mol Gen Genet 240, 428-434 (1993).
21. L. Ortiz-Velez, A. Goodwin, L. Schaefer, R. A. Britton, Challenges and Pitfalls in the Engineering of Human Interleukin 22 (hIL-22) Secreting Lactobacillus reuteri. Frontiers in Bioengineering and Biotechnology 8 (2020).
22. E. Sorvig, G. Mathiesen, K. Naterstad, V. G. H. Eijsink, L. Axelsson, High-level, inducible gene expression in Lactobacillus sakei and Lactobacillus plantarum using versatile expression vectors. Microbiology 151, 2439-2449 (2005).
23. M. Matheu et al., Imaging of effector memory T cells during a delayed-type hypersensitivity reaction and suppression by Kv1.3 channel block. Immunity 29, 602-614 (2008).
24. E. J. Tarcha et al., Durable pharmacological responses from the peptide ShK-186, a specific Kv1.3 channel inhibitor that suppresses T cell mediators of autoimmune diseases. J. Pharmacol. Exp. Ther. 342, 642-653 (2012).
25. C. Beeton, K. G. Chandy, Induction and monitoring of active delayed type hypersensitivity (DTH) in rats. J Vis Exp 10.3791/237, 237 (2007).
26. T. Caon, L. Jin, C. M. Simões, R. S. Norton, J. A. Nicolazzo, Enhancing the buccal mucosal delivery of peptide and protein therapeutics. Pharm Res 32, 1-21 (2015).
27. Y. Chen et al., Genetically engineered oncolytic bacteria as drug delivery systems for targeted cancer theranostics. Acta Biomaterialia 124, 72-87 (2021).
28. P. Praveschotinunt et al., Engineered E. coli Nissle 1917 for the delivery of matrix-tethered therapeutic domains to the gut. Nat Commun 10, 5580 (2019).
29. L. He et al., Escherichia coli Nissle 1917 engineered to express Tum-5 can restrain murine melanoma growth. Oncotarget 8, 85772-85782 (2017).
30. M. K. Puurunen et al., Safety and pharmacodynamics of an engineered E. coli Nissle for the treatment of phenylketonuria: a first-in-human phase 1/2a study. Nature Metabolism 3, 1125-1132 (2021).
31. L. Jin et al., Pulmonary Delivery of the Kv1.3-Blocking Peptide HsTX1[R14A] for the Treatment of Autoimmune Diseases. J Pharm Sci 105, 650-656 (2016).
32. N. J. Bitto, M. Kaparakis-Liaskos, The Therapeutic Benefit of Bacterial Membrane Vesicles. Int J Mol Sci 18 (2017).
33. L. R. McCabe, R. Irwin, L. Schaefer, R. A. Britton, Probiotic use decreases intestinal inflammation and increases bone density in healthy male but not female mice. J Cell Physiol 228, 1793-1798 (2013).
34. J. H. Shim, Z. Stavre, E. M. Gravallese, Bone Loss in Rheumatoid Arthritis: Basic Mechanisms and Clinical Implications. Calcif Tissue Int 102, 533-546 (2018).
35. R. A. Britton et al., Probiotic L. reuteri treatment prevents bone loss in a menopausal ovariectomized mouse model. J. Cell. Physiol. 229, 1822-1830 (2014).
36. F. L. Collins, N. D. Rios-Arce, J. D. Schepper, N. Parameswaran, L. R. McCabe, The Potential of Probiotics as a Therapy for Osteoporosis. Microbiology spectrum 5, 10.1128/microbiolspec.BAD-0015-2016 (2017).
37. D. Quach, N. Parameswaran, L. McCabe, R. A. Britton, Characterizing how probiotic Lactobacillus reuteri 6475 and lactobacillic acid mediate suppression of osteoclast differentiation. Bone Rep 11, 100227-100227 (2019).
38. M. M. Zaiss, H.-J. Joyce Wu, D. Mauro, G. Schett, F. Ciccia, The gut-joint axis in rheumatoid arthritis. Nature Reviews Rheumatology 17, 224-237 (2021).
39. S. Ong et al., Modulation of lymphocyte potassium channel KV1.3 by membrane-penetrating, joint-targeting immunomodulatory plant defensin. ACS Pharmacol. Transl. Sci. 3, 720-736 (2020).
40. R. Bergmann et al., Distribution and kinetics of the Kv1.3-blocking peptide HsTX1[R14A] in experimental rats. Sci Rep 7, 3756 (2017).
41. C. A. Galea, H. M. Nguyen, K. George Chandy, B. J. Smith, R. S. Norton, Domain structure and function of matrix metalloprotease 23 (MMP23): role in potassium channel trafficking. Cell Mol Life Sci 71, 1191-1210 (2014).
42. H. Wulff, H. G. Knaus, M. Pennington, K. G. Chandy, K+ channel expression during B cell differentiation: implications for immunomodulation and autoimmunity. J Immunol 173, 776-786 (2004).
43. R. A. Moura et al., B-cell phenotype and IgD−CD27− memory B cells are affected by TNF-inhibitors and tocilizumab treatment in rheumatoid arthritis. PloS one 12, e0182927-e0182927 (2017).
44. M. H. Rashid et al., A potent and Kv1.3-selective analogue of the scorpion toxin HsTX1 as a potential therapeutic for autoimmune diseases. Sci. Rep. 4, 4509 (2014).
45. S. Grissmer et al., Pharmacological characterization of five cloned voltage-gated K+ channels, types Kv1.1, 1.2, 1.3, 1.5, and 3.1, stably expressed in mammalian cell lines. Mol. Pharmacol. 45, 1227-1234 (1994).
46. M. R. Tanner et al., KCa1.1 and Kv1.3 channels regulate the interactions between fibroblast-like synoviocytes and T lymphocytes during rheumatoid arthritis. Arthritis Res. Ther. 21 (2019).
47. M. R. Tanner et al., Prolonged immunomodulation in inflammatory arthritis using the selective Kv1.3 channel blocker HsTX1[R14A] and its PEGylated analog. Clin. Immunol. 180, 45-57 (2017).
48. C. Beeton, K. G. Chandy, Drawing blood from the saphenous vein of rats and by cardiac puncture. J. Vis. Exp. 7, e266 (2007).
49. C. Beeton, K. G. Chandy, Induction and monitoring of adoptive delayed-type hypersensitivity in rats. J Vis Exp 10.3791/325 (2007).
50. M. Tanner et al., Targeting KCa1.1 channels with a scorpion venom peptide for the therapy of rat models of rheumatoid arthritis. J Pharmacol Exp Ther 365 (2018).
51. M. Brenner et al., Identification of two new arthritis severity loci that regulate levels of autoantibodies, interleukin-1β, and joint damage in pristane- and collagen-induced arthritis. Arthritis Rheum 64, 1369-1378 (2012).

1. A recombinant microorganism expressing one or more biologically active peptides that are heterologous to the microorganism, wherein the one or more biologically active peptides comprise one or more potassium channel blockers.

2. The recombinant microorganism of claim 1, wherein the microorganism is a non-pathogenic microorganism, a probiotic microorganism, and/or a gram-positive bacterium.

3. (canceled)

4. (canceled)

5. The recombinant microorganism of claim 1, wherein the microorganism is a Lactobacillus species strain.

6. The recombinant microorganism of claim 1, wherein the microorganism is a Lactobacillus reuteri strain.

7. The recombinant microorganism of claim 1, wherein the microorganism is Lactobacillus reuteri ATCC PTA 6475 or a derivative thereof.

8. The recombinant microorganism of claim 1, wherein the microorganism is Lactobacillus reuteri LJ01.

9. The recombinant microorganism of claim 1, wherein the one or more biologically active peptides inhibit one or more potassium channels upregulated by activated immune cells and/or one or more potassium channels upregulated by activated fibroblasts and/or fibroblast-like cells.

10. (canceled)

11. The recombinant microorganism of claim 9, wherein the potassium channel comprises a Kv1.3 potassium channel upregulated by activated immune cells, and wherein the more biologically active peptides comprise dalazatide or one or more derivatives thereof.

12. The recombinant microorganism of claim 11, wherein the one or more derivatives of dalazatide comprise ShK-235.

13-15. (canceled)

16. The recombinant microorganism of claim 9, wherein the one or more potassium channels comprise a KCa1.1 potassium channel upregulated by activated fibroblasts and/or fibroblast-like cells, and wherein the one or more biologically active peptides comprise iberiotoxin or one or more derivatives thereof.

17. (canceled)

18. The recombinant microorganism of claim 1, wherein the one or more biologically active peptides are secreted by the recombinant microorganism.

19. A method of generating the recombinant microorganism of claim 1, the method comprising introducing into the microorganism one or more polynucleotides encoding the one or more biologically active peptides.

20. (canceled)

21. (canceled)

22. The method of claim 19, wherein the one or more polynucleotides encoding the one or more biologically active peptides comprise a secretory signal sequence upstream of the one or more biologically active polypeptides.

23. The method of claim 22, wherein the secretory signal sequence comprises a usp45 secretion peptide having at least 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO:1, SEQ ID NO:2, SEQ ID NO:3, SEQ ID NO:4, SEQ ID NO:5, SEQ ID NO:6, or SEQ ID NO:7.

24-45. (canceled)

46. A method of treating or preventing one or more diseases or conditions mediated by cells of the immune system and/or fibroblasts or fibroblast-like cells, the method comprising administering to a subject in need thereof a therapeutically effective amount of the recombinant microorganism of claim 1 or a composition thereof, wherein the recombinant microorganism is capable of producing a therapeutically effective amount of the one or more biologically active polypeptides to treat or prevent the one or more diseases or conditions mediated by cells of the immune system and/or fibroblasts or fibroblast-like cells.

47. The method of claim 46, wherein the one or more biologically active polypeptides produced by the recombinant microorganism are secreted by the recombinant microorganism in situ in the gastrointestinal tract of the individual and enter systemic circulation of the individual.

48-51. (canceled)

52. The method of claim 46, wherein the one or more diseases or conditions mediated by cells of the immune system and/or fibroblasts or fibroblast-like cells are characterized by inflammation caused at least in part by proliferation and/or activation of immune cells and/or fibroblasts or fibroblast-like cells,
wherein the one or more biologically active polypeptides secreted by the recombinant microorganism inhibit the proliferation and/or activation of immune cells and/or fibroblasts or fibroblast-like cells, and
wherein administration of one or more biologically active polypeptides reduces inflammation.

53-59. (canceled)

60. The method of claim 46, wherein the one or more diseases or conditions mediated by cells of the immune system and/or fibroblasts or fibroblast-like cells comprise an inflammatory disease, an autoimmune disease, an alloimmune disease, or an allergic disease.

61-70. (canceled)

71. The method of claim 70, wherein the recombinant microorganism is orally or rectally administered in a dose of at least about 1×10⁷ CFU to at least about 1×10¹² CFU of the recombinant microorganism at least once per day.

72. (canceled)

73. The method of claim 46, wherein the recombinant microorganism of claim 1 or the composition thereof is administered in combination with one or more additional therapeutics comprising one or more nonsteroidal anti-inflammatory drugs, one or more corticosteroids, one or more disease-modifying anti-rheumatic drugs, or one or more antibodies targeted to cytokines or cytokine receptors.

74. (canceled)


SEQUENCE LISTING (12 sequences)

SEQ ID NO: 1 - protein, 29 aa, synthetic construct
MEKKIISAILMSTVILSAAAPLSGVYAAA

SEQ ID NO: 2 - protein, 27 aa, synthetic construct
MKKKIISAILMSTVILSAAAPLSGVYA

SEQ ID NO: 3 - protein, 27 aa, synthetic construct
MKKKIISAILMSTVILSAAAPLSGVYA

SEQ ID NO: 4 - protein, 40 aa, synthetic construct
MKKFNFKTMLLLVLASCVFGVVVNVTTSLGPQTAITAQAS

SEQ ID NO: 5 - protein, 44 aa, synthetic construct
MNRQPNNDPSTHFKMYKSGKKWVFAGLTAVTLLTASGAVAHADD

SEQ ID NO: 6 - protein, 32 aa, synthetic construct
MKLSKKVAKITAAITGAVALGTVATATTANAD

SEQ ID NO: 7 - protein, 35 aa, synthetic construct
MGKFWKKALLTIAALTVGTSAGITSVSAASSAVNX

SEQ ID NO: 8 - DNA, 6055 bp, synthetic construct
  source 1..6055: mol_type = other DNA
atggagaagaaaattatttcagctattttaatgtcaactgttattttatcagctgcagctccattatcaggtgtttatgctgctgcacgaagttgtattgatacgattccaaaatcacggtgtaccgcttttaaatgcaagcacagtattaagtaccgtttgtcattctgccggaaaacttgtggaacttgtgcttaagaattcggtaccccgggttcgaaggcgccaagcttcaaattacagcacgtgttgctttgattgatagccaaaaagcagcagttgataaagcaattactgatattgctgaaaaattgtaatttataaataaaaatcaccttttagaggtggtttttttatttataaattattcgtttgatttcgctttcgatagaacaatcaaagcgagaataaggaagataaatcccataagggcgggagcagaatgtccgagactaattcatgagatcgattttttattaaaacgtctcaaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgtccttctatttcggttggaggaggctcaagggagtttgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttgaggggtttttcaatcgccaacgaatcgccaacgttttcgccaacgttttttataaatctatatttaagtagctttattgttgtttttatgattacaaagtgatacactaattttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagcccccctgacgaaagtcgaagggggcttttattttggtttgatgttgcgattaatagcaatacgattgcaataaacaaaacgatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggggtcgacccttcaatagagttcttaacgttaatccgaaaaaaactaacgttaatattaaaaaataagatccgcttgtgaattatgtataatttgattagactaaagaataggagaaagtatgatgatatttaaaaaactttctcgttaagataggttgttggtgagcatgttatatacggatgtatcggtttccttaatgcaaaattttgttgctatcttattaatttttctattatatagatatattcaaagaaagataacatttaaacggatcatattagatattttaatagcgattattttttcaatattatatctgtttatttcagatgcgtcattacttgtaatggtattaatgcgattagggtggcattttcatcaacaaaaagaaaataagataaaaacgactgatacagctaatttaattctaattatcgtgatccagttattgttagttgcggttgggactattattagtcagtttaccatatcgattatcaaaagtgatttcagccaaaatatattgaacaatagtgcaacagatataactttattaggtattttctttgctgttttatttgacggcttgttctttatattattgaagaataagcggactgaattacaacatttaaatcaagaaatcattgaattttcgttagaaaaacaatattttatatttatatttattttatttatagtaatagaaattattttagcagttgggaatcttcaaggagtaacagccacgatattattaaccattatcattattttttgtgtccttatcgggatgactttttggcaagtgatgctttttttgaaggcttattcgattcgccaagaagccaatgaccaattggtccggaatcaacaacttcaagattatctagtcaatatcgaacagcagtacaccgaattacggcgatttaagcatgattatcaaaacatcttattatcgttggagagttttgccgaaaagggcgatcagcaacagtttaaggcgtattaccaagaattattagcacaacggccaattcaaagtgaaatccaaggggcagtcattgcacaactcgactacttgaaaaatgatcctattcgaggattagtcattcaaaagtttttggcagccaaacaggctggtgttactttaaaattcgaaatgaccgaaccaatcgaattagcaaccgctaatctattaacggttattcggattatcggtattttattagacaatgcgattgaacaagccgttcaagaaaccgatcaattggtgagttgtgctttcttacaatctgatggtttaatcgaaattacgattgaaaatacggccagtcaagttaagaatctccaagcattttcagagttaggctattcaacgaaaggcgctggtcgggggactggtttagctaatgtgcaggatttgattgccaaacaaaccaatttattcttagaaacacagattgaaaatagaaagttacgacagacattgatgattacggaggaaacttaatttgtatcccgtttatttattagaggatgatttacagcaacaagcgatttatcagcaaattatcgcgaatacgattatgattaacgaatttgcaatgactttaacatgcgctgccagtgatactgagacattgttggcggcaattaaggatcagcaacgaggtttattctttttggatatggaaattgaggataaccgccaagccggtttagaagtggcaactaagattcggcagatgatgccgtttgcgcaaattgtcttcattacaacccacgaggaactgacattattaacgttagaacgaaaaatagcgcctttagattacattctcaaggaccaaacaatggctgaaatcaaaaggcaattgattgatgatctattgttagctgagaagcaaaacgaggcggcagcgtatcaccgagaaaatttatttagttataaaataggtcctcgctttttctcattaccattaaaggaagttgtttatttatatactgaaaaagaaaatccgggtcatattaatttgttagccgttaccagaaaggttacttttccaggaaatttaaatgcgctggaagcccaatatccaatgctctttcggtgtgataaaagttacttagttaacctatctaatattgccaattatgacagtaaaacacggagtttaaaatttgtagatggcagtgaggcaaaagtctcgttccggaaatcacgggaactagtggccaaattaaaacaaatgatgtagcgcctgcaggcacgccaaatgatcccagtaaaaagccacccgcatggcgggtggctttttattagccctagaagggcttcccacacgcatttcagcgccttagtgccttagtttgtgaatcataggtggtatagtcccgaaatacccgtctaaggaattgtcagataggcctaatgactggcttttataatatgagataatgccgactgtactttttacagtcggttttctaatgtcactaacctgccccgttagttgaagaaggtttttatattacagctccagatctaccggtttaatttgaaaattgatattagcgtttaacagttaaattaatacgttaataatttttttgtctttaaatagggatttgaagcataatggtgttatagcgtacttagctggccagcatatatgtattctataaaatactattacaaggagattttagcc

SEQ ID NO: 9 - DNA, 6058 bp, synthetic construct
  source 1..6058: mol_type = other DNA
aaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggggtcgacccttcaatagagttcttaacgttaatccgaaaaaaactaacgttaatattaaaaaataagatccgcttgtgaattatgtataatttgattagactaaagaataggagaaagtatgatgatatttaaaaaactttctcgttaagataggttgttggtgagcatgttatatacggatgtatcggtttccttaatgcaaaattttgttgctatcttattaatttttctattatatagatatattcaaagaaagataacatttaaacggatcatattagatattttaatagcgattattttttcaatattatatctgtttatttcagatgcgtcattacttgtaatggtattaatgcgattagggtggcattttcatcaacaaaaagaaaataagataaaaacgactgatacagctaatttaattctaattatcgtgatccagttattgttagttgcggttgggactattattagtcagtttaccatatcgattatcaaaagtgatttcagccaaaatatattgaacaatagtgcaacagatataactttattaggtattttctttgctgttttatttgacggcttgttctttatattattgaagaataagcggactgaattacaacatttaaatcaagaaatcattgaattttcgttagaaaaacaatattttatatttatatttattttatttatagtaatagaaattattttagcagttgggaatcttcaaggagtaacagccacgatattattaaccattatcattattttttgtgtccttatcgggatgactttttggcaagtgatgctttttttgaaggcttattcgattcgccaagaagccaatgaccaattggtccggaatcaacaacttcaagattatctagtcaatatcgaacagcagtacaccgaattacggcgatttaagcatgattatcaaaacatcttattatcgttggagagttttgccgaaaagggcgatcagcaacagtttaaggcgtattaccaagaattattagcacaacggccaattcaaagtgaaatccaaggggcagtcattgcacaactcgactacttgaaaaatgatcctattcgaggattagtcattcaaaagtttttggcagccaaacaggctggtgttactttaaaattcgaaatgaccgaaccaatcgaattagcaaccgctaatctattaacggttattcggattatcggtattttattagacaatgcgattgaacaagccgttcaagaaaccgatcaattggtgagttgtgctttcttacaatctgatggtttaatcgaaattacgattgaaaatacggccagtcaagttaagaatctccaagcattttcagagttaggctattcaacgaaaggcgctggtcgggggactggtttagctaatgtgcaggatttgattgccaaacaaaccaatttattcttagaaacacagattgaaaatagaaagttacgacagacattgatgattacggaggaaacttaatttgtatcccgtttatttattagaggatgatttacagcaacaagcgatttatcagcaaattatcgcgaatacgattatgattaacgaatttgcaatgactttaacatgcgctgccagtgatactgagacattgttggcggcaattaaggatcagcaacgaggtttattctttttggatatggaaattgaggataaccgccaagccggtttagaagtggcaactaagattcggcagatgatgccgtttgcgcaaattgtcttcattacaacccacgaggaactgacattattaacgttagaacgaaaaatagcgcctttagattacattctcaaggaccaaacaatggctgaaatcaaaaggcaattgattgatgatctattgttagctgagaagcaaaacgaggcggcagcgtatcaccgagaaaatttatttagttataaaataggtcctcgctttttctcattaccattaaaggaagttgtttatttatatactgaaaaagaaaatccgggtcatattaatttgttagccgttaccagaaaggttacttttccaggaaatttaaatgcgctggaagcccaatatccaatgctctttcggtgtgataaaagttacttagttaacctatctaatattgccaattatgacagtaaaacacggagtttaaaatttgtagatggcagtgaggcaaaagtctcgttccggaaatcacgggaactagtggccaaattaaaacaaatgatgtagcgcctgcaggcacgccaaatgatcccagtaaaaagccacccgcatggcgggtggctttttattagccctagaagggcttcccacacgcatttcagcgccttagtgccttagtttgtgaatcataggtggtatagtcccgaaatacccgtctaaggaattgtcagataggcctaatgactggcttttataatatgagataatgccgactgtactttttacagtcggttttctaatgtcactaacctgccccgttagttgaagaaggtttttatattacagctccagatctaccggtttaatttgaaaattgatattagcgtttaacagttaaattaatacgttaataatttttttgtctttaaatagggatttgaagcataatggtgttatagcgtacttagctggccagcatatatgtattctataaaatactattacaaggagattttagccatggagaagaaaattatttcagctattttaatgtcaactgttattttatcagctgcagctccattatcaggtgtttatgctgctgcagaatttactgatgttgattgtagtgttagtaaagaatgttggagtgtttgtaaagatttatttggtgttgatcgtggtaaatgtatgggtaaaaaatgtcgttgttatcaataagaattcggtaccccgggttcgaaggcgccaagcttcaaattacagcacgtgttgctttgattgatagccaaaaagcagcagttgataaagcaattactgatattgctgaaaaattgtaatttataaataaaaatcaccttttagaggtggtttttttatttataaattattcgtttgatttcgctttcgatagaacaatcaaagcgagaataaggaagataaatcccataagggcgggagcagaatgtccgagactaattcatgagatcgattttttattaaaacgtctcaaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgtccttctatttcggttggaggaggctcaagggagtttgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttgaggggtttttcaatcgccaacgaatcgccaacgttttcgccaacgttttttataaatctatatttaagtagctttattgttgtttttatgattacaaagtgatacactaattttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagcccccctgacgaaagtcgaagggggcttttattttggtttgatgttgcgattaatagcaatacgattgcaataaacaaaacgatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaacca

SEQ ID NO: 10 - protein, 35 aa, synthetic construct
RSCIDTIPKSRCTAFQCKHSMKYRLSFCRKTCGTC

SEQ ID NO: 11 - protein, 43 aa, synthetic construct
PTYRAEEARSCIDTIPKSRCTAFQCKHSMKYRLSFCRKTCGTC

SEQ ID NO: 12 - protein, 36 aa, synthetic construct
RSCIDTIPKSRCTAFKCKHSIKYRLSFCRKTCGTCA